(19) United States
(12) Patent Application Publication
(10) Pub. No.: US 2012/0245176 A1
(43) Pub. Date: Sep. 27, 2012

(54) NOVEL PIPERAZINE ANALOGS WITH SUBSTITUTED HETEROARYL GROUPS AS BROAD-SPECTRUM INFLUENZA ANTIVIRALS

(75) Inventors: Christopher W. Cianci, Madison, CT (US); Samuel Gerritz, Guilford, CT (US); Sean Kim, Southington, CT (US); David R. Langley, Meriden, CT (US); Guo Li, Wallingford, CT (US); Bradley C. Pearce, East Hampton, CT (US); Annapurna Pendri, South Glastonbury, CT (US); Shuhao Shi, Madison, CT (US); Weixu Zhai, Middletown, CT (US); Shirong Zhu, Cheshire, CT (US)

(73) Assignee: Bristol-Myers Squibb Company

(21) Appl. No.: 13/242,676

(22) Filed: Sep. 23, 2011

Related U.S. Application Data
(60) Provisional application No. 61/387,186, filed on Sep. 28, 2010.

(51) Int. Cl. A61K 31/496; C07D 413/14; A61K 31/506; A61P 31/16; C07D 403/06; C07D 401/14; C07D 417/06; A61P 31/12; C07D 413/06; A61K 31/498

(52) U.S. Cl. 514/249; 544/367; 514/254.04; 544/364; 514/253.1; 544/363; 514/253.06; 544/295; 514/252.19; 514/253.05; 544/353; 544/371; 514/254.05; 544/366; 544/372; 514/254.01; 544/370; 514/253.09

(57) ABSTRACT

A compound of Formula I is set forth, including pharmaceutically acceptable salts thereof:

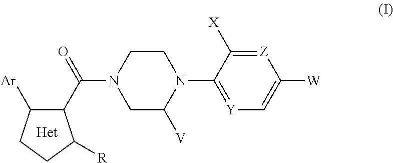

wherein Het is a 5 or 6-membered heterocycle with —N, —O, or —S adjacent to the —Ar substituent or adjacent to the point of attachment for the —Ar substituent;
Ar is aryl or heteroaryl;
R is —CH₃, —CH₂F, —CHF₂ or —CH═CH₂;
V is —H, —CH₃ or ═O;
W is —NO₂, —Cl, —Br, —CH₂OH, or —CN;
X is —Cl, —Br, —F, —CH₃, —OCH₃, or —CN;
Y is —CH or —N; and
Z is —CH or —N.

This compound is useful in compositions for the prevention and treatment of influenza virus.

### CROSS REFERENCE TO RELATED APPLICATION

This non-provisional application claims the benefit of U.S. Provisional Application Ser. No. 61/387,186 filed Sep. 28, 2010.

### FIELD OF THE INVENTION

The present invention relates to novel piperazine compounds with one or more substituted heteroaryl groups, useful for the prophylaxis and treatment of influenza virus, and to compositions and formulations containing these compounds. The invention also relates to methods for preventing and treating influenza infection utilizing the compounds herein set forth.

### BACKGROUND OF THE INVENTION

Influenza virus is a significant causative agent of acute lower respiratory tract infections in humans. It transmits readily, resulting in annual epidemics that can manifest in severe illness and death for high-risk populations. It is one of the RNA viruses of the family Orthomyxoviridae that affects birds and mammals, and is responsible for the illness commonly referred to as the “flu”. The most common symptoms of the flu are chills, fever, sore throat, muscle pains, severe headache, coughing, weakness/fatigue and general discomfort. Sore throat, fever and coughs are the most frequent symptoms. In more serious cases, influenza causes pneumonia, which can be fatal, particularly for the young and the elderly. Although it is often confused with other influenza-like illnesses, especially the common cold, influenza is a more severe disease than the common cold and is caused by a different type of virus. Influenza may produce nausea and vomiting, particularly in children, but these symptoms are more common in the unrelated gastroenteritis, which is sometimes called “stomach flu” or “24-hour flu”.

Typically, the influenza virus is transmitted through the air by coughs or sneezes, creating aerosols containing the virus. Influenza can also be transmitted by direct contact with bird droppings or nasal secretions, or through contact with contaminated surfaces. Airborne aerosols have been thought to cause most infections, although which means of transmission is most important is not absolutely clear.

Influenza remains a constant threat, as new variants emerge seasonally. Annual epidemics take an economic toll through lost workforce productivity, while straining health service resources. Additionally, influenza virus is responsible for major pandemics every 10-50 years. In 2009, a new H1N1 triple re-assortment of swine influenza emerged in North America and reached pandemic proportion (Zimmer and Burke, 2009). Influenza virus' ability to mutate (antigenic drift), as well as re-assort with other influenza viruses from different mammalian species (antigenic shift), are mechanisms causing seasonal epidemic variation and pandemic virus insurgence, respectively (Chen and Deng, 2009). Moreover, resistance to available anti-influenza agents is increasing. The majority of H3N2 isolates and 2009 H1N1 are resistant to the adamantane M2 ion channel inhibitors (Deyde et al, 2009). Furthermore, 2008 H1N1 has shown resistance to the neuraminidase inhibitor Tamiflu (Oseltamivir), the standard of care (Moscona, 2009). Neither class has been shown to be effective against highly pathogenic H5N1 avian virus (Soepandi, 2010).

Multiple novel therapeutic and prophylactic agents against influenza virus are therefore currently needed in the art. Also needed are new compositions and formulations containing these agents, as well as new methods for preventing and treating influenza utilizing these agents.

### SUMMARY OF THE INVENTION

The invention, in a first embodiment, provides a compound of Formula I, including pharmaceutically acceptable salts thereof:

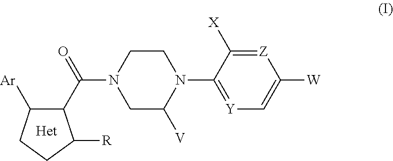

wherein Het is a 5 or 6-membered heterocycle with —N, —O, or —S adjacent to the —Ar substituent or adjacent to the point of attachment for the —Ar substituent;
Ar is aryl or heteroaryl;

### R is —CH₃, —CH₂F, —CHF₂ or —CH═CH₂;

### V is —H, —CH₃ or ═O;

### W is —NO₂, —Cl, —Br, —CH₂OH, or —CN;

### X is —Cl, —Br, —F, —CH₃, —OCH₃, or —CN;

### Y is —CH or —N; and

### Z is —CH or —N;

with the proviso that the compound of Formula I does not include the following compounds:

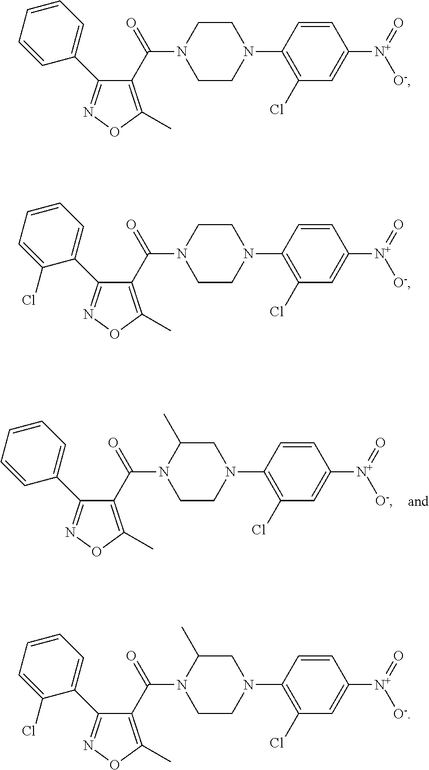

Also provided as part of the invention is a pharmaceutical composition which comprises an antiviral effective amount of one or more of the compounds of Formula I, including pharmaceutically acceptable salts thereof, together with one or more pharmaceutically acceptable carriers, excipients or diluents.

In addition, there is provided a method for treating a mammal infected with influenza virus comprising administering to said mammal an antiviral effective amount of a compound of Formula I including pharmaceutically acceptable salts thereof, and one or more pharmaceutically acceptable carriers, excipients or diluents.

Methods for making the compounds of Formula I are also herein provided.

The invention is directed to these and other important ends, hereinafter described.

### DETAILED DESCRIPTION OF THE EMBODIMENTS

Since the compounds of the present invention may possess asymmetric centers and therefore occur as mixtures of diastereomers and enantiomers, the present invention includes the individual diastereoisomeric and enantiomeric forms of the compounds of Formula I in addition to the mixtures thereof.

### DEFINITIONS

Unless otherwise specifically set forth elsewhere in the application, one or more of the following terms may be used herein, and shall have the following meanings:

The term “C₁₋₆ alkyl” as used herein means straight or branched chain alkyl groups such as methyl, ethyl, propyl, isopropyl, butyl, isobutyl, t-butyl, amyl, hexyl and the like.

“Halogen” refers to chlorine, bromine, iodine or fluorine.

“H” or “Hydrogen” refers to hydrogen, including its isotopes such as deuterium.

An “aryl” group refers to an all carbon monocyclic or fused-ring polycyclic(i.e., rings which share adjacent pairs of carbon atoms) groups having a completely conjugated pi-electron system. Examples, without limitation, of aryl groups are phenyl, napthalenyl and anthracenyl. The aryl group may be substituted or unsubstituted. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino and —NR^{x}R^{y}, wherein R^{x} and R^{y} are independently selected from the group consisting of hydrogen, alkyl, cycloalkyl, aryl, carbonyl, C-carboxy, sulfonyl, trihalomethyl, and, combined, a five- or six-member heteroalicyclic ring.

As used herein, a “heteroaryl” group refers to a monocyclic or fused ring (i.e., rings which share an adjacent pair of atoms) group having in the ring(s) one or more atoms selected from the group consisting of nitrogen, oxygen and sulfur and, in addition, having a completely conjugated pi-electron system. Unless otherwise indicated, the heteroaryl group may be attached at either a carbon or nitrogen atom within the heteroaryl group. It should be noted that the term heteroaryl is intended to encompass an N-oxide of the parent heteroaryl if such an N-oxide is chemically feasible as is known in the art. Examples, without limitation, of heteroaryl groups are furyl, thienyl, benzothienyl, thiazolyl, imidazolyl, oxazolyl, oxadiazolyl, thiadiazolyl, benzothiazolyl, triazolyl, tetrazolyl, isoxazolyl, isothiazolyl, pyrrolyl, pyranyl, tetrahydropyranyl, pyrazolyl, pyridyl, pyrimidinyl, quinolinyl, isoquinolinyl, purinyl, carbazolyl, benzoxazolyl, benzimidazolyl, indolyl, isoindolyl, pyrazinyl, diazinyl, pyrazine, triazinyl, tetrazinyl, and tetrazolyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thioalkoxy, thiohydroxy, thioaryloxy, thioheteroaryloxy, thioheteroalicycloxy, cyano, halogen, nitro, carbonyl, O-carbamyl, N-carbamyl, C-amido, N-amido, C-carboxy, O-carboxy, sulfinyl, sulfonyl, sulfonamido, trihalomethyl, ureido, amino, and —NR^{x}R^{y}, wherein R^{x} and R^{y} are as defined above.

As used herein, a “heteroalicyclic” group refers to a monocyclic or fused ring group having in the ring(s) one or more atoms selected from the group consisting of nitrogen, oxygen and sulfur. Rings are selected from those which provide stable arrangements of bonds and are not intended to encompass systems which would not exist. The rings may also have one or more double bonds. However, the rings do not have a completely conjugated pi-electron system. Examples, without limitation, of heteroalicyclic groups are azetidinyl, piperidyl, piperazinyl, imidazolinyl, thiazolidinyl, 3-pyrrolidin-1-yl, morpholinyl, thiomorpholinyl and tetrahydropyranyl. When substituted the substituted group(s) is preferably one or more selected from alkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halogen, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, silyl, guanyl, guanidino, ureido, phosphonyl, amino and —NR^{x}R^{y}, wherein R^{x} and R^{y} are as defined above.

An “alkyl” group refers to a saturated aliphatic hydrocarbon including straight chain and branched chain groups. Preferably, the alkyl group has 1 to 20 carbon atoms (whenever a numerical range; e.g., “1-20”, is stated herein, it means that the group, in this case the alkyl group may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The alkyl group may be substituted or unsubstituted. When substituted, the substituent group(s) is preferably one or more individually selected from trihaloalkyl, cycloalkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalomethanesulfonamido, trihalomethanesulfonyl, and combined, a five- or six-member heteroalicyclic ring.

A “cycloalkyl” group refers to an all-carbon monocyclic or fused ring (i.e., rings which share and adjacent pair of carbon atoms) group wherein one or more rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane. A cycloalkyl group may be substituted or unsubstituted. When substituted, the substituent group(s) is preferably one or more individually selected from alkyl, aryl, heteroaryl, heteroalicyclic, hydroxy, alkoxy, aryloxy, heteroaryloxy, heteroalicycloxy, thiohydroxy, thioalkoxy, thioaryloxy, thioheteroaryloxy, thioheteroalicycloxy, cyano, halo, nitro, carbonyl, thiocarbonyl, O-carbamyl, N-carbamyl, O-thiocarbamyl, N-thiocarbamyl, C-amido, C-thioamido, N-amido, C-carboxy, O-carboxy, sulfinyl, sulfonyl, sulfonamido, trihalo-methanesulfonamido, trihalomethanesulfonyl, silyl, guanyl, guanidino, ureido, phosphonyl, amino and −NR^{x}R^{y} with R^{x} and R^{y} as defined above.

An “alkenyl” group refers to an alkyl group, as defined herein, having at least two carbon atoms and at least one carbon-carbon double bond.

An “alkynyl” group refers to an alkyl group, as defined herein, having at least two carbon atoms and at least one carbon-carbon triple bond.

A “hydroxy” group refers to an —OH group.

An “alkoxy” group refers to both an —O-alkyl and an —O-cycloalkyl group as defined herein.

An “aryloxy” group refers to both an —O-aryl and an —O-heteroaryl group, as defined herein.

A “heteroaryloxy” group refers to a heteroaryl-O— group with heteroaryl as defined herein.

A “heteroalicycloxy” group refers to a heteroalicyclic-O— group with heteroalicyclic as defined herein.

A “thiohydroxy” group refers to an —SH group.

A “thioalkoxy” group refers to both an S-alkyl and an —S-cycloalkyl group, as defined herein.

A “thioaryloxy” group refers to both an —S-aryl and an —S-heteroaryl group, as defined herein.

A “thioheteroaryloxy” group refers to a heteroaryl-S— group with heteroaryl as defined herein.

A “thioheteroalicycloxy” group refers to a heteroalicyclic-S— group with heteroalicyclic as defined herein.

A “carbonyl” group refers to a —C(═O)—R″ group, where R″ is selected from the group consisting of hydrogen, alkyl, alkenyl, alkynyl, cycloalkyl, aryl, heteroaryl (bonded through a ring carbon) and heteroalicyclic (bonded through a ring carbon), as each is defined herein.

An “aldehyde” group refers to a carbonyl group where R″ is hydrogen.

A “thiocarbonyl” group refers to a —C(═S)—R″ group, with R″ as defined herein.

A “Keto” group refers to a —CC(═O)C— group wherein the carbon on either or both sides of the C═O may be alkyl, cycloalkyl, aryl or a carbon of a heteroaryl or heteroalicyclic group.

A “trihalomethanecarbonyl” group refers to a Z₃CC(═O)— group with said Z being a halogen.

A “C-carboxy” group refers to a —C(═O)O—R″ groups, with R″ as defined herein.

An “O-carboxy” group refers to a R″C(—O)O-group, with R″ as defined herein.

A “carboxylic acid” group refers to a C-carboxy group in which R″ is hydrogen.

A “trihalomethyl” group refers to a —CZ₃, group wherein Z is a halogen group as defined herein.

A “trihalomethanesulfonyl” group refers to an Z₃CS(═O)₂— groups with Z as defined above.

A “trihalomethanesulfonamido” group refers to a Z₃CS(═O)₂NR^{x}— group with Z as defined above and R^{x} being H or (C₁₋₆)alkyl.

A “sulfinyl” group refers to a —S(═O)—R″ group, with R″ being (C₁₋₆)alkyl.

A “sulfonyl” group refers to a —S(═O)₂R″ group with R″ being (C₁₋₆)alkyl.

A “S-sulfonamido” group refers to a —S(═O)₂NR^{X}R^{Y}, with R^{X} and R^{Y} independently being H or (C₁₋₆)alkyl.

A “N-Sulfonamido” group refers to a R″S(═O)₂NR_{X}— group, with Rₓ being H or (C₁₋₆)alkyl;

A “O-carbamyl” group refers to a —OC(═O)NR^{x}R^{y} group, with R^{X} and R^{Y} independently being H or (C₁₋₆)alkyl.

A “N-carbamyl” group refers to a R^{x}OC(═O)NR^{y} group, with R^{x} and R^{y} independently being H or (C₁₋₆)alkyl.

A “O-thiocarbamyl” group refers to a —OC(═S)NR^{x}R^{y} group, with R^{x} and R^{y} independently being H or (C₁₋₆)alkyl.

A “N-thiocarbamyl” group refers to a R^{x}OC(═S)NR^{y}— group, with R^{x} and R^{y} independently being H or (C₁₋₆)alkyl.

An “amino” group refers to an —NH₂ group.

An “amido” group refers to a univalent radical —NH₂ when attached via a carboxyl group.

A “C-amido” group refers to a —C(═O)NR^{x}R^{y} group, with R^{x} and R^{y} independently being H or (C₁₋₆)alkyl.

A “C-thioamido” group refers to a —C(═S)NR^{x}R^{y} group, with R^{x} and R^{y} independently being H or (C₁₋₆)alkyl.

A “N-amido” group refers to a R^{x}C(═O)NR^{y}— group, with R^{x} and R^{y} independently being H or (C₁₋₆)alkyl.

An “ureido” group refers to a —NR^{x}C(═O)NR^{y}R^{y2} group, with R^{x}, R^{y}, and R^{y2} independently being H or (C₁₋₆)alkyl.

A “guanidino” group refers to a —R^{x}NC(═N)NR^{y}R^{y2} group, with R^{x}, R^{y}, and R^{y2} independently being H or (C₁₋₆)alkyl.

A “guanyl” group refers to a R^{x}R^{y}NC(═N)— group, with R^{x} and R^{y} independently being H or (C₁₋₆)alkyl.

A “cyano” group refers to a —CN group.

A “silyl” group refers to a —Si(R″)₃, with R″ being (C₁₋₆)alkyl or phenyl.

A “phosphonyl” group refers to a P(═O)(OR^{x})₂ with R^{x} being (C₁₋₆)alkyl.

A “hydrazino” group refers to a —NR^{x}NR^{y}R^{y2} group, with R^{x}, R^{y}, and R^{y2} independently being H or (C₁₋₆)alkyl.

Any two adjacent R groups may combine to form an additional aryl, cycloalkyl, heteroaryl or heterocyclic ring fused to the ring initially bearing those R groups.

It is known in the art that nitrogen atoms in heteroaryl systems can be “participating in a heteroaryl ring double bond”, and this refers to the form of double bonds in the two tautomeric structures which comprise five-member ring heteroaryl groups. This dictates whether nitrogens can be substituted as well understood by chemists in the art. The disclosure and claims of the present disclosure are based on the known general principles of chemical bonding. It is understood that the claims do not encompass structures known to be unstable or not able to exist based on the literature.

Physiologically acceptable salts and prodrugs of compounds disclosed herein are within the scope of this disclosure. The term “pharmaceutically acceptable salt” as used herein and in the claims is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine.

As set forth above, the present invention is directed to compounds of Formula I, including pharmaceutically acceptable salts thereof:

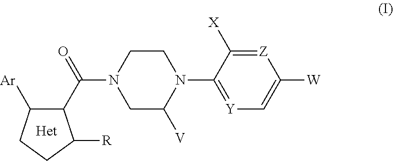

wherein Het is a 5 or 6-membered heterocycle with —N, —O, or —S adjacent to the —Ar substituent or adjacent to the point of attachment for the —Ar substituent;
Ar is aryl or heteroaryl;

### R is —CH₃, —CH₂F, —CHF₂ or —CH═CH₂;

### V is —H, —CH₃ or ═O;

### W is —NO₂, —Cl, —Br, —CH₂OH, or —CN;

### X is —Cl, —Br, —F, —CH₃, —OCH₃, or —CN;

### Y is —CH or —N; and

### Z is —CH or —N;

with the proviso that the compound of Formula I does not include the following compounds:

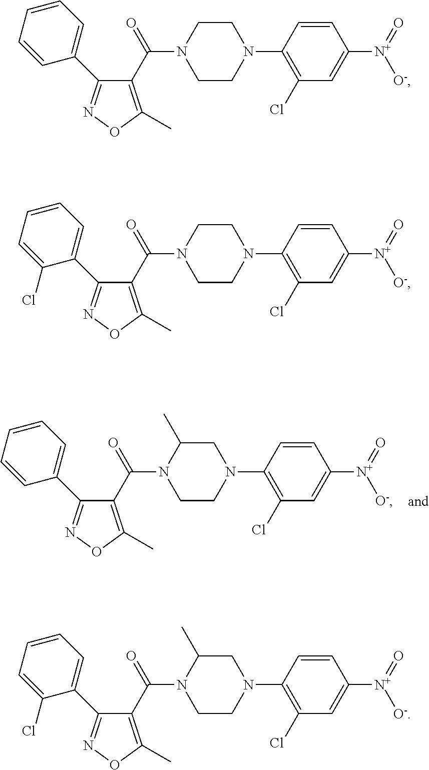

In a preferred embodiment of the invention, the substituent Het is selected from the group of:

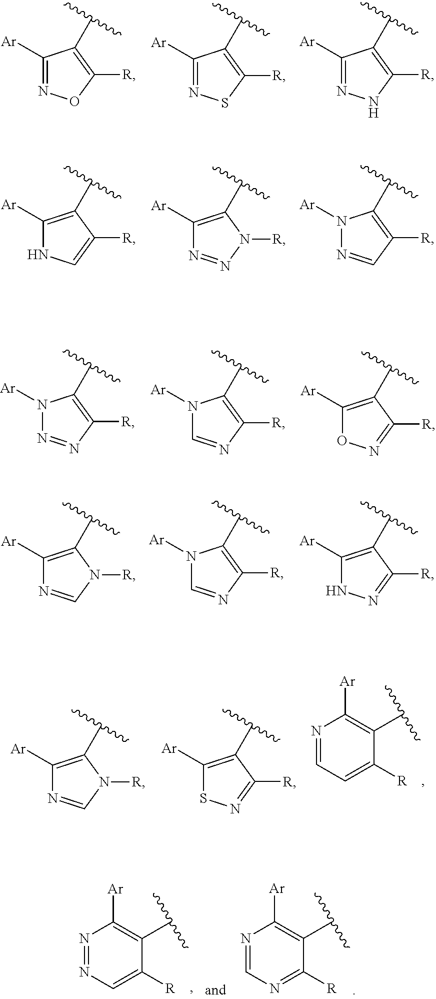

In particular, it is preferred that Het is a 5 or 6-membered heterocycle with —N adjacent to the point of attachment for the —Ar component. Even more preferably, Het is selected from the group of:

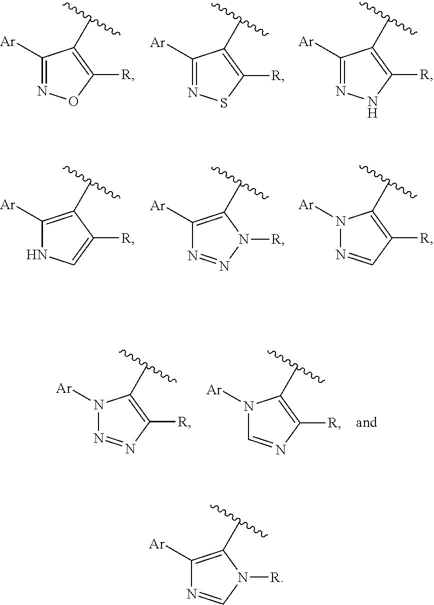

Of the foregoing, the Het substituents

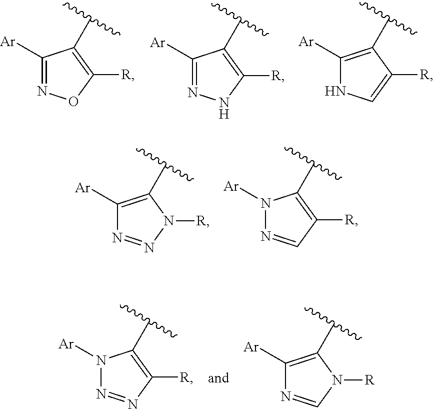

are particularly preferred.

In a further embodiment of the compounds of Formula I, it is preferred that Ar is selected from the group of:

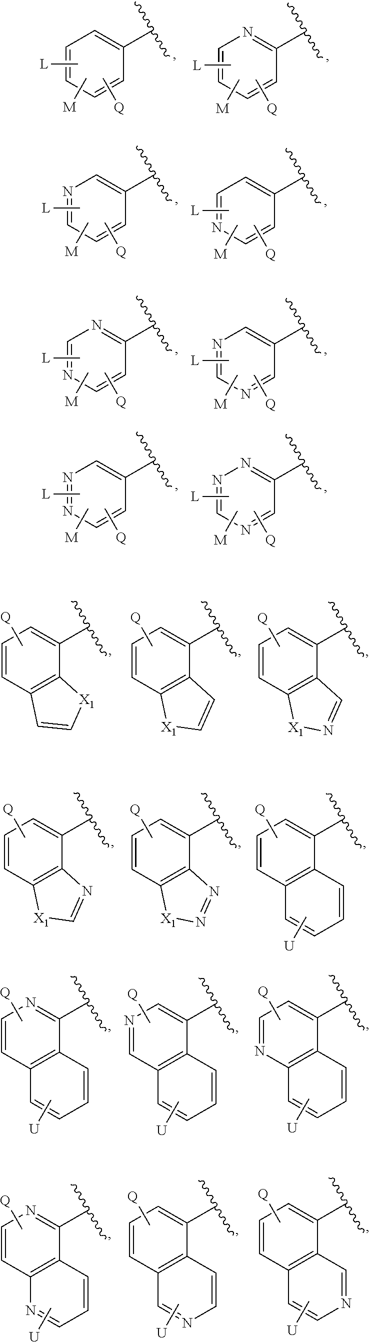

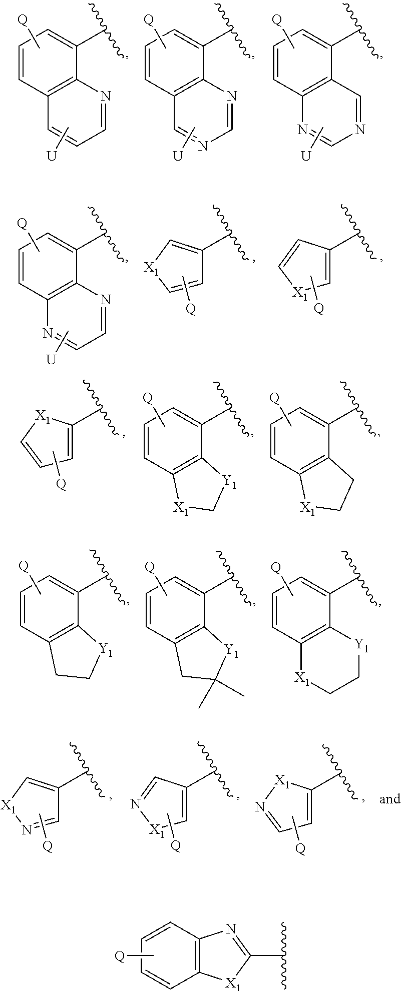

wherein
L is H, halogen, cyano, hydroxyl, amino, alkyl, alkoxy, alkylamino, or amido;
M is H, halogen, cyano, hydroxyl, amino, alkyl, alkoxy, alkylamino, or amido;
Q is H, halogen, cyano, hydroxyl, amino, alkyl, alkoxy, alkylamino, or amido;
U is H, halogen, cyano, hydroxyl, amino, alkyl, alkoxy, alkylamino, or amido;

### X₁ is O, NH, N-alkyl, N-aryl, S or CH₂; and

### Y₁ is O, NH, N-alkyl, N-aryl, S or CH₂.

Even more preferably, the Ar substituent is selected from the group of:

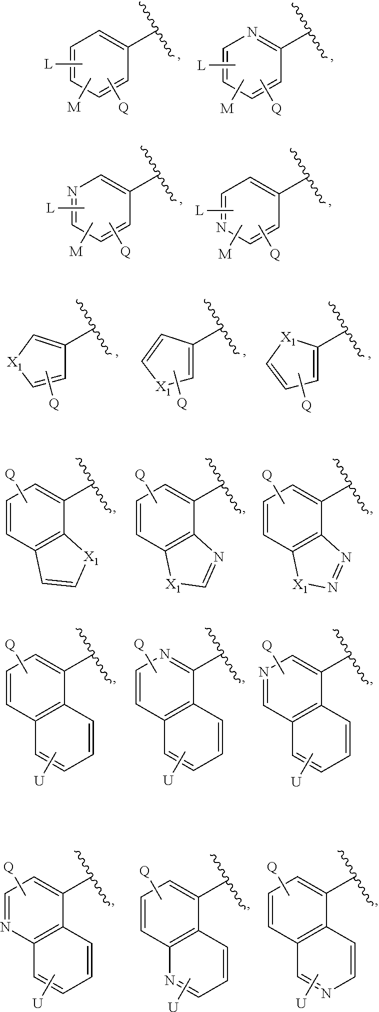

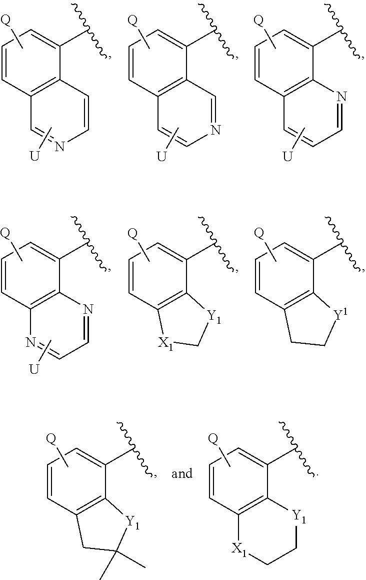

It is even more preferred that Ar be selected from the group of:

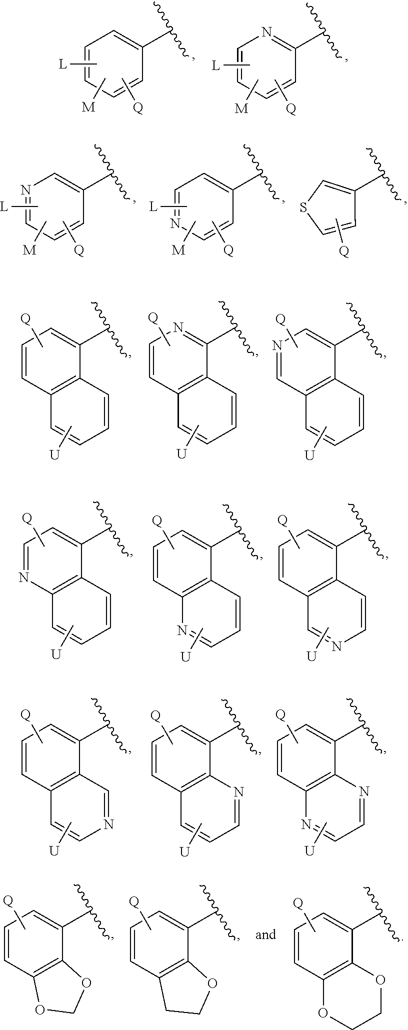

It is especially preferred that Ar be a phenyl group, or phenyl which is substituted with methoxy or hydroxyl. In other embodiments, Ar may be a fused bicyclic structure.

As set forth above, the substituent R is —CH₃, —CH₂F, or —CH═CH₂. Preferably, R is —CH₃ or —CH₂F. Even more preferably, R is —CH₃.

The substituent V is preferably —H.

The substituent W is defined as being selected from the group of —NO₂, —Cl, —Br, —CHO, —CH═CH₂, and —CN. More preferably, W is —NO₂, —Cl, —Br, or —CN. It is especially preferred that W be —NO₂, —Cl, or —Br, with —NO₂ or —Br being even more preferred.

The substituent X is —Cl, —CH₃, or —CN. Even more preferably, X is —Cl or —CN, with —Cl being even more preferred.

The substituent Y can be —CH or —N. In certain embodiments, it is preferred that Y be —CH. It certain other embodiments, it is preferred both that Y be —CH and that the Ar substituent be phenyl which is substituted with either methoxy or a hydroxyl group.

The substituent Z is preferably —CH.

Preferred compounds of Formula I, including pharmaceutically acceptable salts thereof, include the following:

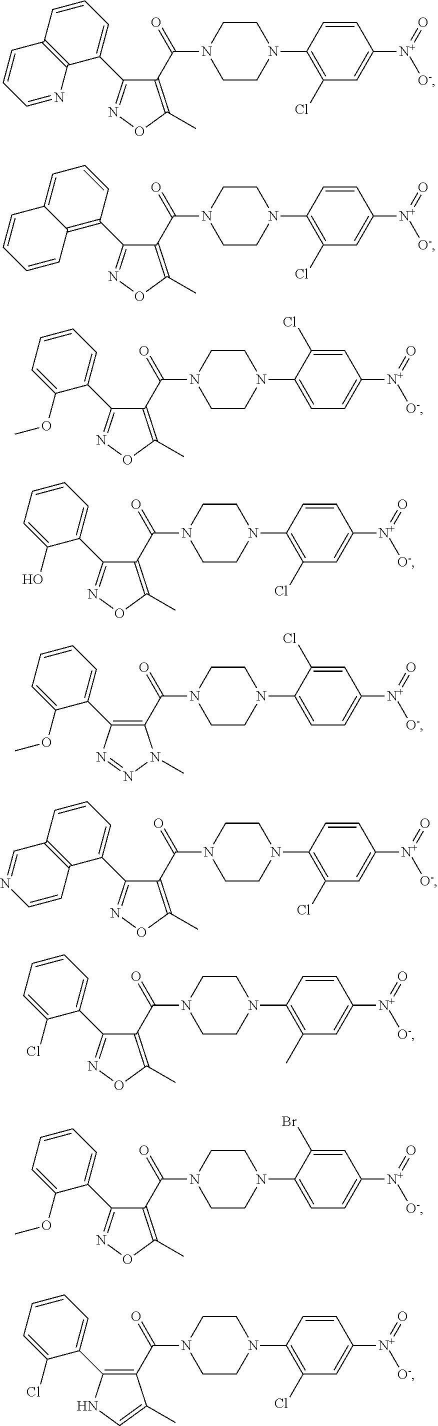

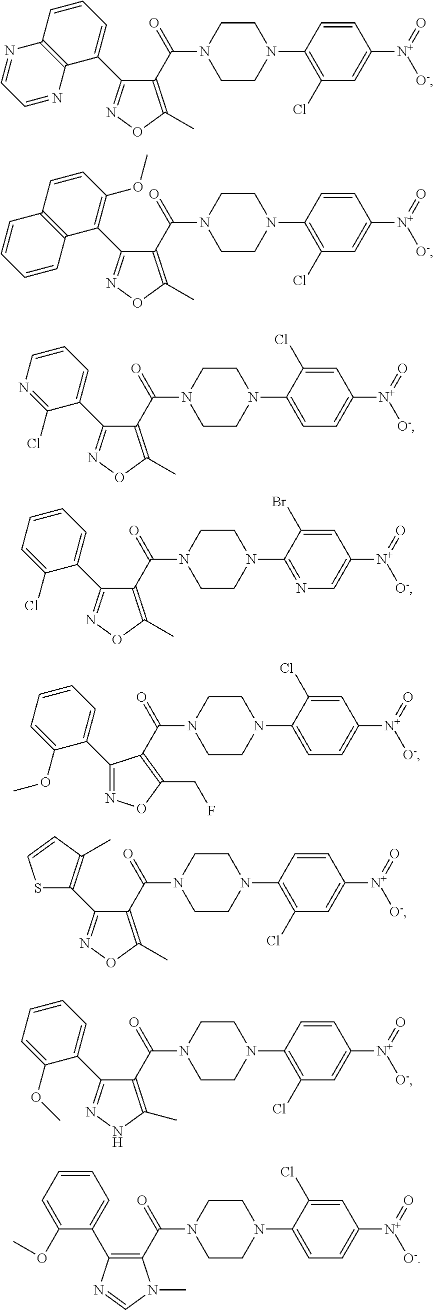

The compounds of the present invention may be administered orally, parenterally (including subcutaneous injections, intravenous, intramuscular, intrasternal injection or infusion techniques), by inhalation spray, or rectally or by other means available in the art, in dosage unit formulations containing conventional non-toxic pharmaceutically acceptable carriers, adjuvants and diluents.

Thus, in accordance with the present invention, there is further provided a method of treating and a pharmaceutical composition for treating viral infections such as influenza infection. The treatment involves administering to a patient in need of such treatment a pharmaceutical composition comprising a pharmaceutical carrier and a therapeutically effective amount of a compound of the present disclosure.

The pharmaceutical composition may be in the form of orally administrable suspensions or tablets; nasal sprays, sterile injectable preparations, for example, as sterile injectable aqueous or oleaginous suspensions or suppositories.

When administered orally as a suspension, these compositions are prepared according to techniques available in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art.

The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The compounds herein set forth can be administered orally to humans in a dosage range of 1 to 100 mg/kg body weight, perhaps in divided doses. One preferred dosage range is 1 to 10 mg/kg body weight orally in divided doses. Another preferred dosage range is 1 to 20 mg/kg body weight in divided doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In the compositions and methods of the present invention herein described, the term “antiviral effective amount” means the total amount of each active compound or component of the composition or method that is sufficient to show a meaningful patient benefit, e.g., prevention of infection by influenza or healing of acute conditions or symptoms characterized by influenza infection. The terms “treat, treating, treatment” as used herein and in the claims means preventing or ameliorating diseases and symptoms associated with influenza infection. When applied to an individual active ingredient, administered alone, the term refers to that ingredient alone. When applied to a combination, the term refers to combined amounts of the active ingredients that result in the therapeutic effect, whether administered in combination, serially or simultaneously.

The present invention is also directed to combinations of the compounds herein described with one or more other agents useful in the treatment of influenza. For example, the compounds of this invention may be effectively administered, whether at periods of pre-exposure and/or post-exposure, in combination with effective amounts of other influenza antivirals, immunomodulators, antiinfectives, or vaccines available in the art.

The following schemata are generalized procedures for making the compounds of the invention by those skilled in the art.

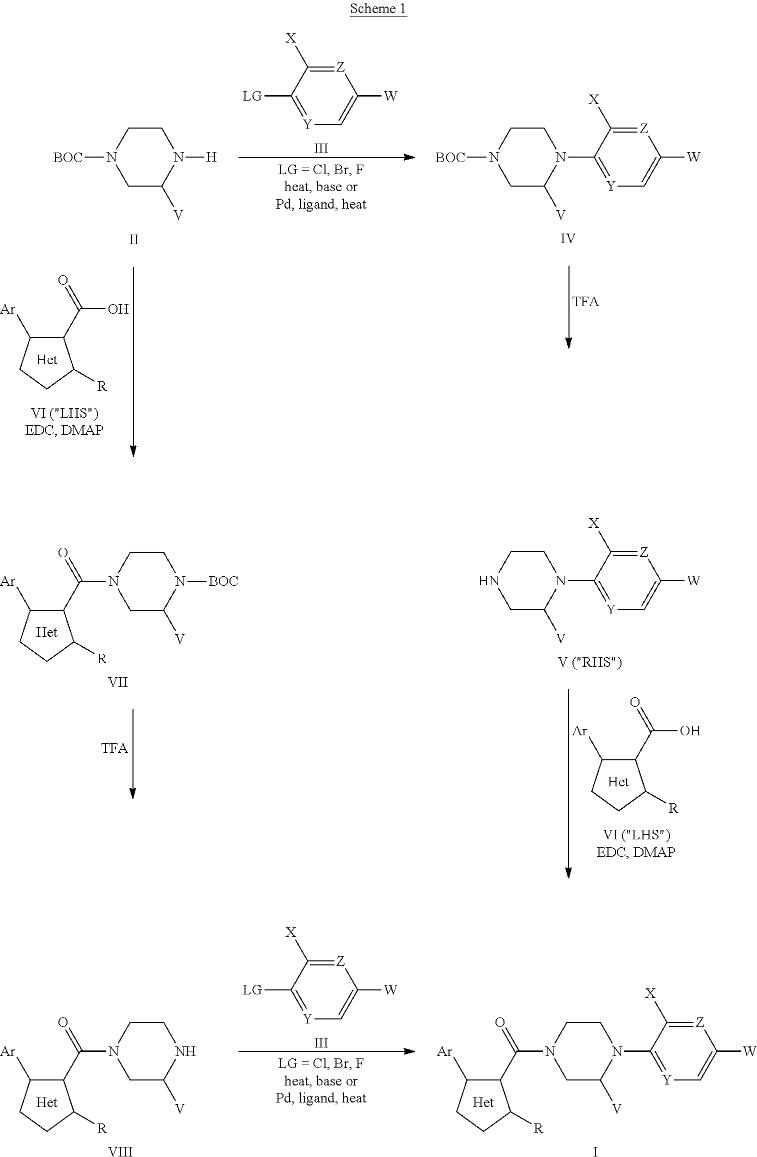

Compounds of formula I were prepared from intermediates of formula II via two complementary routes as illustrated in Scheme 1. In the first route, intermediates of formula II were treated with intermediates of formula III in the presence of base and heat or in the presence of base, heat and a palladium catalyst to afford intermediates of formula IV. Intermediates of formula III can be obtained commercially, can be prepared by methods known in the literature, or can be readily prepared by one skilled in the art. Intermediates of formula IV were treated with TFA to provide intermediates of formula V (also referred to as the “RHS” or right hand side). Intermediates of formula V were treated with carboxylic acids of formula VI (also referred to as the “LHS” or left hand side) and an amide-bond forming reagent (i.e. EDC) to provide compounds of formula I. Carboxylic acids of formula IV can be obtained commercially, can be prepared by methods known in the literature, or can be readily prepared by one skilled in the art. In the second route, the synthetic steps described in the first route are reversed. Intermediates of formula II were treated with carboxylic acids of formula VI (also referred to as the “LHS” or left hand side) and an amide-bond forming reagent to afford intermediates of formula VII. Carboxylic acids of formula VI can be obtained commercially, can be prepared by methods known in the literature, or can be readily prepared by one skilled in the art. Intermediates of formula VII were treated with trifluoroacetic acid to afford intermediates of formula VIII. Intermediates of formula VIII were treated with intermediates of formula III in the presence of base and heat or in the presence of base, heat and a palladium catalyst to afford compounds of formula I. Intermediates of formula III can be obtained commercially, can be prepared by methods known in the literature, or can be readily prepared by one skilled in the art.

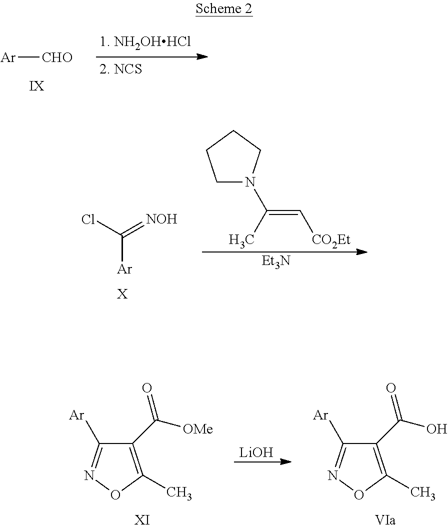

Compounds of formula VIa were prepared as outlined in Scheme 2 and as described in the literature. [See: Gerald W. Zamponi, Stephanie C. Stotz, Richard J. Staples, Tina M. Andro, Jared K. Nelson, Victoria Hulubei, Alex Blumenfeld, and Nicholas R. Natale, J. Med. Chem., 2003, 46, 87-96.] Sequential treatment of aryl aldehyde derivatives of formula IX with hydroxylamine hydrochloride, then n-chlorosuccinimide provided intermediates of formula X. Aldehyde derivatives of formula IX can be obtained commercially, can be prepared by methods known in the literature, or can be readily prepared by one skilled in the art. Intermediates of formula XI were prepared by treatment of chlorooximes of formula XII with (E)-ethyl 3-(pyrrolidin-1-yl)but-2-enoate to afford isoxazoles of formula XI. Hydrolysis of the methyl ester of isoxazoles of formula XI afforded compounds of formula VIa.

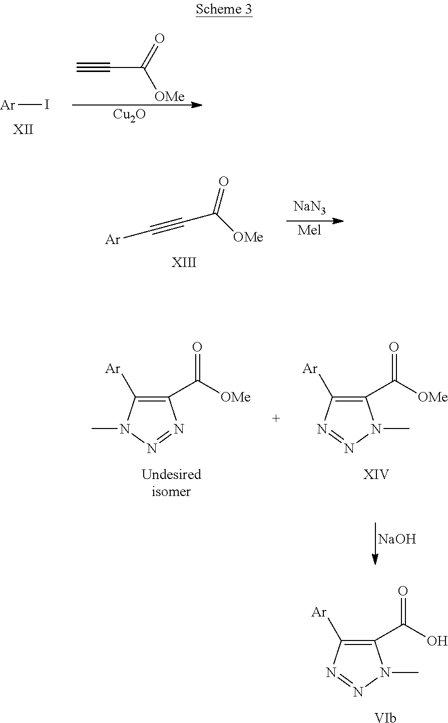

Compounds of formula VIb were prepared as outlined in Scheme 3. Aryl iodides of formula XII were coupled with methyl propiolate in the presence of copper (I) oxide [See: Liliebris, C.; Larsen, S. D; Ogg, D.; Palazuk, B. J; and Pleasdale, J. E. J. Med. Chem., 2002, 45, 1785.] to provide intermediates of formula XIII Aryl iodide derivatives of formula XII can be obtained commercially, can be prepared by methods known in the literature, or can be readily prepared by one skilled in the art. Intermediates of formula XIII were treated with sodium azide and methyl iodide to afford triazoles of formula XIV after chromatographic separation from an undesired regioisomer. Treatment of triazoles of formula XIV with sodium hydroxide provided compounds of formula VIb.

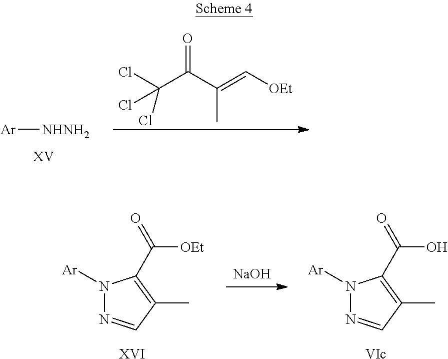

Compounds of formula IVc were prepared as outlined in Scheme 4 and as described in the literature. [See: Martins, M. A. P. et al. J. Molecular Catalysis A: Chemical, 2007, 266, 100.] Aryl hydrazines of formula XV were treated with (E)-1,1,1-trichloro-4-ethoxy-3-methylbut-3-en-2-one to afford pyrazoles of formula XVI after chromatographic separation from the undesired regioisomer. Aryl hydrazines of formula XV can be obtained commercially, can be prepared by methods known in the literature, or can be readily prepared by one skilled in the art. Treatment of pyrazoles of formula XVI with sodium hydroxide provided compounds of formula VIc.

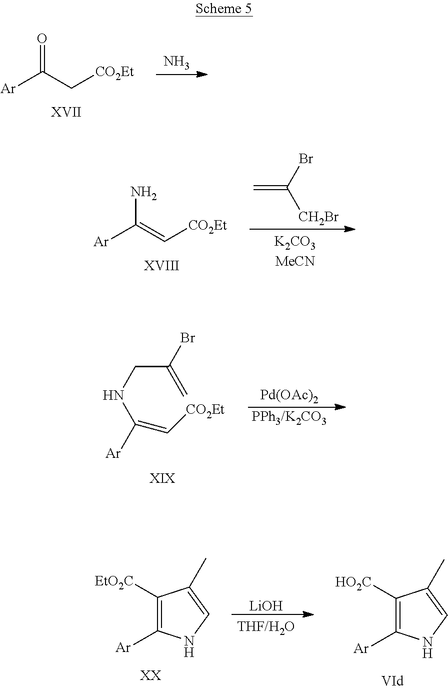

Compounds of formula VId were prepared as outlined in Scheme 5 and as described in the literature. [See: Grigg, R.; Savic, V. Chem. Commun. 2000, (10), 873-874.] Beta-ketoesters of formula XVII were treated with ammonia to afford enamines of formula XVIII. Beta-ketoesters of formula XVII can be obtained commercially, can be prepared by methods known in the literature, or can be readily prepared by one skilled in the art. Treatment of enamines of formula XVIII with 2,3-dibromoprop-1-ene provided intermediates of formula XIX, which upon treatment with palladium(II) acetate afforded pyrroles of formula XX. Treatment of pyrroles of formula XX with lithium hydroxide provided compounds of formula VId.

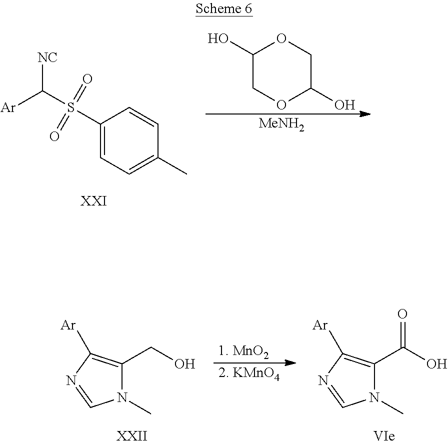

Compounds of formula VIe were prepared as outlined in Scheme 6 and as described in the literature. [See: Luke, R. W. A.; Jones, C. D.; McCoull, W; Hayter, B. R. WO Patent 2004013141, 2004.] Isocyanates of formula XXI were treated with 1,4-dioxane-2,5-diol and methylamine to afford imidazoles of formula XXII. Isocyanates of formula XXI can be obtained commercially, can be prepared by methods known in the literature, or can be readily prepared by one skilled in the art. Sequential treatment of imidazoles of formula XXII with manganese (IV) oxide and potassium permanganate afforded compounds of formula VIe.

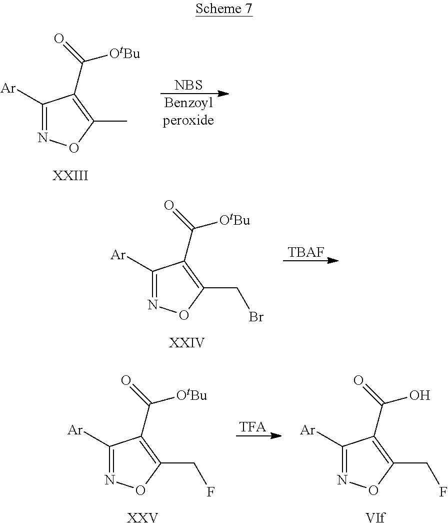

Compounds of formula VIf were prepared as outlined in Scheme 7. Isoxazoles of formula XXIII were brominated under free radical conditions to provide intermediates of formula XXIV. Isoxazoles of formula XXIV can be obtained commercially, can be prepared by methods known in the literature, can be prepared by analogy to Scheme 2, or can be readily prepared by one skilled in the art. Intermediates of formula XXIV were treated with tetrabutylammonium fluoride to afford intermediates of formula XXV. [See: Sun, H.; DiMagno, S. G., J. Am. Chem. Soc. 2005, 127, 2050-2051.] Treatment of intermediates of formula XXV with trifluoroacetic acid provided compounds of formula VIf.

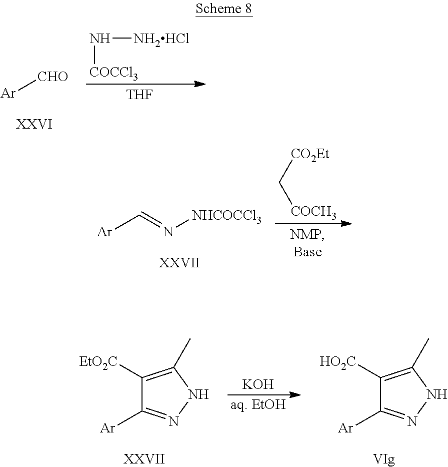

Compounds of formula VIg were prepared as outlined in Scheme 8 and as described in the literature. [See: El Kaim, L.; Lacroix, S. Synlett, 2000, 3, 353-354.] Aldehydes of formula XXVI were condensed with trichloroacetylhydrazide to afford hydrazones of formula XXVII. Aldehydes of formula XXVI can be obtained commercially, can be prepared by methods known in the literature, or can be readily prepared by one skilled in the art. Treatment of hydrazones of formula XXVII with ethyl acetoacetate under basic conditions afforded pyrazoles of formula XXVIII, which were hydrolyzed in a subsequent step to afford compounds of formula VIg.

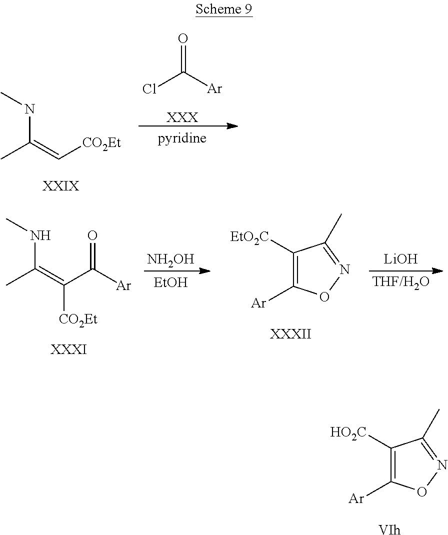

Compounds of formula VIh were prepared as outlined in Scheme 9 and as described in the literature. [See: Chantegrel, B.; Nadi, A. I.; Gelin, S. J. Org. Chem., 1984, 49, 4419-4424.] Enamine of formula XXIX was treated with acid chlorides of formula XXX to afford enamines of formula XXXI. Acid chlorides of formula XXX can be obtained commercially, can be prepared by methods known in the literature, or can be readily prepared by one skilled in the art. Treatment of enamines of formula XXXI with hydroxylamine provided isoxazoles of formula XXXII. Treatment of isoxazoles of formula XXXII with lithium hydroxide provided compounds of formula VIh.

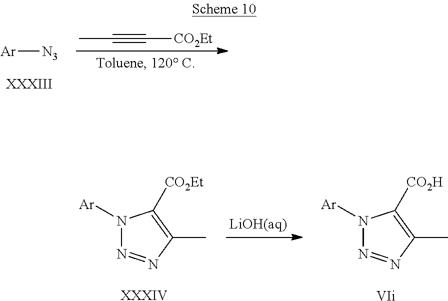

Compounds of formula VIi were prepared as outlined in Scheme 10 and as described in the literature. [See: Bell, M. G. et al. PCT Int. Appl. 2007, WO 2007140174.] Aryl azides of formula XXXIII were treated with ethyl but-2-ynoate to afford triazoles of formula XXXIV. Aryl azides of formula XXXIII can be obtained commercially, can be prepared by methods known in the literature, or can be readily prepared by one skilled in the art. Treatment of triazoles of formula XXXIV with lithium hydroxide provided compounds of formula VIi.

### EXAMPLES

The compounds herein described and set forth and their preparation can be understood further by the following working examples. These examples are meant to be illustrative of the present invention, and are not to be taken as limiting the scope thereof.

Chemical abbreviations used in the Examples are defined as follows:
“Ac” for acetate,
“APCI” for atmospheric pressure chemical ionization,
“BEMP” for 2-tert-butimino-2-diethylamino-1,3-dimethyl-perhydro-1,3,2-diaza-phosphorine,
“Boc” or “BOC” for t-butyloxycarbonyl,
“BOP” for benzotriazol-1-yloxytris-(dimethylamino)-phosphonium hexafluorophosphate,
“Cbz” for benzyloxycarbonyl,
“CDI” for 1,1′-carbonyldiimidazole,
“CD₃OD” for deuteromethanol,
“CDCl₃” for deuterochloroform,
“DCC” for 1,3-dicyclohexylcarbodiimide,
“DCE” for 1,2-dichloroethane,
“DCM” for dichloromethane
“DEAD” for diethyl azodicarboxylate,
“DIEA”, “Hunig's base”, or “DIPEA” for N,N-diisopropylethylamine,
“DMF” for N,N-dimethylformamide,
“DMAP” for 4-dimethylaminopyridine,
“DMPU” for 1,3-dimethyl-3,4,5,6-tetrahydro-2(1H)-pyrimidone,
“DMSO” for dimethylsulfoxide,
“DPPA” for diphenylphosphorylazide
“EDC” or “EDCI” for 1-(3-dimethylaminopropyl)-3-ethylcarbodiimide hydrochloride,
“Et” for ethyl,
“EtOAC” for ethyl acetate,
“HOAc” for acetic acid,
“HOBt” for 1-hydroxybenzotriazole hydrate,
“HATU” for O-(7-azabenzotriazol-1-yl)-N,N,N′,N′-tetramethyluronium hexafluorophosphate,
“HMPA” for hexamethylphosphoramide,
“LDA” for lithium diisopropylamide,
“LiHMDS” for lithium bis(trimethylsilyl)amide,
“NaHMDS” for sodium bis(trimethylsilyl)amide,
“NBS” for N-bromosuccinimide,
“NCS” for N-chlorosuccinimide,
“NMM” for 4-methylmorpholine,
“PyBOP” for benzotriazole-1-yl-oxy-tris-pyrrolidino-phosphonium hexafluorophosphate,
“TMSCH₂N₂” for (trimethylsilyl)diazomethane,
“TMSN₃” for Azidotrimethylsilane,
“TBTU” for O-(1H-benzotriazol-1-yl)-N,N,N′,N′-tetramethyluronium tetrafluoroborate,
“TEA” for triethylamine,
“TFA” for trifluoroacetic acid, and
“THF” for tetrahydrofuran.

Abbreviations used in the Examples are defined as follows: “° C.” for degrees Celsius, “MS” for mass spectrometry, “ESI” for electrospray ionization mass spectroscopy, “HR” for high resolution, “LC-MS” for liquid chromatography mass spectrometry, “eq” for equivalent or equivalents, “g” for gram or grams, “h” for hour or hours, “mg” for milligram or milligrams, “mL” for milliliter or milliliters, “mmol” for millimolar, “M” for molar, “min” for minute or minutes, “rt” for room temperature, “NMR” for nuclear magnetic resonance spectroscopy, “tlc” for thin layer chromatography, “atm” for atmosphere, and “α”, “β”, “R”, “S”, “E”, and “Z” are stereochemical designations familiar to one skilled in the art.

“HPLC” is an abbreviation used herein for high pressure liquid chromatography. Reverse-phase HPLC can be carried out using a Vydac C-18 column with gradient elution from 10% to 100% buffer B in buffer A (buffer A: water containing 0.1% trifluoroacetic acid, buffer B: 10% water, 90% acetonitrile containing 0.1% trifluoroacetic acid). If necessary, organic layers can be dried over sodium sulfate unless otherwise indicated. However, unless otherwise indicated, the following conditions are generally applicable. “LC-MS” refers to high pressure liquid chromatography carried out according to the definition for HPLC with a mass spectrometry detector.

| | | | Gradient | | | | | |
|---|---|---|---|---|---|---|---|---|
| | | | Time | Flow rate | | | | |
| Method | Start % B | Final % B | (min) | (ml/min) | λ | Column | Solvent A | Solvent B |
| A | 0 | 100 | 3 | 4 | 220 | Xterra | 10% MeOH—90% | 90% MeOH—10% |
| | | | | | | 4.6 × 30 mm S5 | H2O—0.1% TFA | H2O—0.1% TFA |
| B | 0 | 100 | 3 | 4 | 220 | Phenomenex-Luna | 5% ACN—95% | 95% ACN—5% |
| | | | | | | 4.6 × 50 mm S5 | H2O—10 mM | H2O—10 mM |
| | | | | | | | NH4Ac | NH4Ac |
| C | 0 | 100 | 3 | 4 | 220 | SunFire C18 5 u | 90% Water/ | 10% Water/ |
| | | | | | | 4.6 × 50 mm | 10% ACN/ | 90% ACN/ |
| | | | | | | | 0.1% TFA | 0.1% TFA |
| D | 0 | 100 | 2 | 4 | 220 | Phenomenex-Luna | 5% ACN—95% | 95% ACN—5% |
| | | | | | | 3.0 × 50 mm S10 | H2O—10 mM | H2O—10 mM |
| | | | | | | | NH4Ac | NH4Ac |
| E | 0 | 100 | 3 | 4 | 220 | Phenomenex-Luna | 10% MeOH—90% | 90% MeOH—10% |
| | | | | | | 3.0 × 50 mm S10 | H2O—0.1% TFA | H2O—0.1% TFA |
| F | 0 | 100 | 3 | 4 | 220 | Luna | 5% ACN—95% | 95% ACN—5% |
| | | | | | | 4.6 × 30 mm S10 | H2O—10 mM | H2O—10 mM |
| | | | | | | | NH4Ac | NH4Ac |
| G | 0 | 100 | 4 | 0.8 | 220 | Phenomenex-Luna, | 90% H2O—10% | 10% H2O—90% |
| | | | | | | 2.0 × 50 mm, 3 u | ACN—0.1% TFA | ACN—0.1% TFA |
| H | 0 | 100 | 3 | 5 | 220 | Phenomenex-Luna | 10% MeOH—90% | 90% MeOH—10% |
| | | | | | | 4.6 × 50 mm S10 | H2O—0.1% TFA | H2O—0.1% TFA |
| I | 0 | 100 | 3 | 4 | 220 | Luna | 5% ACN—95% | 95% ACN—5% |
| | | | | | | 3.0 × 50 mm S10 | H2O—10 mM | H2O—10 mM |
| | | | | | | | NH4Ac | NH4Ac |
| J | 0 | 100 | 2 | 4 | 220 | Phenomenex-Luna | 5% MeOH—95% | 95% MeOH—5% |
| | | | | | | 3.0 × 50 mm S10 | H2O—10 mM | H2O—10 mM |
| | | | | | | | NH4Ac | NH4Ac |
| K | 0 | 100 | 2 | 4 | 254 | Phenomenex-Luna | 90% Water/ | 10% Water/ |
| | | | | | | 3.0 × 50 mm S10 | 10% ACN/ | 90% ACN/ |
| | | | | | | | 0.1% TFA | 0.1% TFA |
| L | 0 | 100 | 2 | 4 | 254 | Phenomenex-Luna | 10% MeOH—90% | 90% MeOH—10% |
| | | | | | | 4.6 × 50 mm S10 | H2O—0.1% TFA | H2O—0.1% TFA |
| M | 0 | 100 | 2 | 4 | 254 | Phenomenex-Luna | 90% Water/ | 10% Water/ |
| | | | | | | 4.6 × 50 mm S10 | 10% ACN/ | 90% ACN/ |
| | | | | | | | 0.1% TFA | 0.1% TFA |
| N | 0 | 100 | 2 | 4 | 254 | Phenomenex-Luna | 10% MeOH—90% | 90% MeOH—10% |
| | | | | | | 3.0 × 50 mm S10 | H2O—0.1% TFA | H2O—0.1% TFA |
| O | 0 | 95 | 15 | 1.2 | 220 | Ascentis C-18, | 5% ACN—95% | 95% ACN—5% |
| | | | | | | 4.6 × 150 mm | H2O—10 mM | H2O—10 mM |
| | | | | | | | NH4Ac | NH4Ac |
| P | 0 | 100 | 4 | 4 | 220 | Gemini | 5% ACN—95% | 95% ACN—5% |
| | | | | | | 4.6 × 50 mm S5 | H2O—10 mM | H2O—10 mM |
| | | | | | | | NH4Ac | NH4Ac |
| Q | 0 | 100 | 10 | 2 | 220 | Waters Xbridge C-18 | 5% ACN—95% | 5% ACN—95% |
| | | | | | | 4.6 × 50 mm | H2O—10 mM | H2O—10 mM |
| | | | | | | | NH4Ac | NH4Ac |
| R | 20 | 95 | 7 | 3 | 220 | Ascentis C-18, | 5% ACN—95% | 95% ACN—5% |
| | | | | | | 4.6 × 50 mm | H2O—10 mM | H2O—10 mM |
| | | | | | | | NH4Ac | NH4Ac |
| S | 0 | 100 | 4 | 4 | 220 | XTERRA | 5% ACN—95% | 95% ACN—5% |
| | | | | | | 3.0 × 50 MM S7 | H2O—10 mM | H2O—10 mM |
| | | | | | | | NH4Ac | NH4Ac |
| T | 0 | 100 | 3 | 4 | 220 | phenomenex C18 | 10% Methanol—90% | 90% Methanol—10% |
| | | | | | | 3.0 × 50 MM | H2O—0.1% TFA | H2O—0.1% TFA |
| U | 30 | 95 | 15 | 20 | 220 | Waters Xbridge C-18 | H2O—10 mM | ACN—10 mM |
| | | | | | | 150 × 19 mm, 5 u | NH4Ac | NH4Ac |
| V | 40 | 95 | 6 | 1.5 | 220 | Phenomenex Gemini | H2O—0.1% TFA | ACN—0.1% TFA |
| | | | | | | 4.6 × 100 mm, 5 u | | |

Preparatory HPLC: When described as performed under “standard conditions”, samples (approx. 20 mg) were dissolved in methanol (10 mg/mL) and purified on a 25 mm×50 mm Vydac C18 column with a 5 minute gradient elution from 10% to 100% buffer B in buffer A (buffer A: water containing 0.1% trifluoroacetic acid, buffer B: 10% water, 90% acetonitrile containing 0.1% trifluoroacetic acid) at 10 mL/minute.

Melting points were determined on a MeI-Temp II apparatus and are uncorrected. IR spectra were obtained on a single-beam Nicolet Nexus FT-IR spectrometer using 16 accumulations at a resolution of 4.00 cm-1 on samples prepared in a pressed disc of KBr or as a film on KBr plates. Proton NMR spectra (300 MHz, referenced to tetramethylsilane) were obtained on a Varian INOUA 300, Bruker Avance 300, Avance 400, or Avance 500 spectrometer. Data were referred to the lock solvent. Electrospray Ionization (ESI) experiments were performed on a Micromass II Platform single-quadrupole mass spectrometer, or on a Finnigan SSQ7000 mass spectrometer.

### Synthesis of Intermediates

### Acid-A. 3-(2-methoxyphenyl)-5-methylisoxazole-4-carboxylic acid

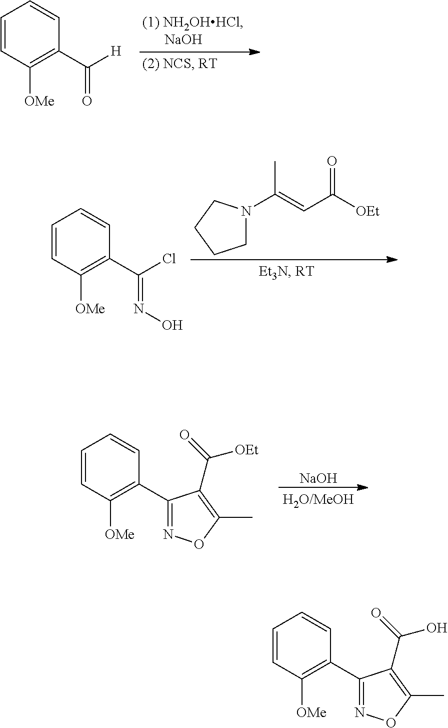

### Step A1

Ethyl 3-(2-methoxyphenyl)-5-methylisoxazole-4-carboxylate was synthesized from 2-methoxybenzaldehyde as described in [Zamponi, G. W.; Stotz, S. C.; Staples, R. J.; Andro, T. M.; Nelson, J. K.; Hulubei, V.; Blumenfeld, A.; Natale, N. R. J. Med. Chem. 2003, 46, 87-96.]

### Step A2

The reaction mixture of ethyl 3-(2-methoxyphenyl)-5-methylisoxazole-4-carboxylate (12.85 g, 49.2 mmol) and sodium hydroxide (9.84 g, 246 mmol) in MeOH (100 mL) and Water (10 mL) in a 500-mL round bottom flask was stirred at 65° C. for 20 hours. The MeOH was removed in vacuo, then the concentrated reaction mixture was transferred to a 500-mL separatory funnel with 150 mL of water and 100 mL of ether. The organic layer was discarded. The aqueous layer was acidified by adding concentrated HCl (26 mL). The product precipitated and was separated by filtration and dried under high vacuum to give 10.63 g (93%, theoretical yield 11.47 g). ¹H NMR (400 MHz, CD₃OD) δ 2.69 (s, 3H, CH₃), 3.77 (s, 3H, OCH₃), 7.00-7.07 (m, 2H, aryl), 7.32-7.34 (m, ¹H, aryl), 7.43-7.48 (m, ¹H, aryl).

Acids B-AJ were synthesized by analogy to Acid-A, substituting the appropriate aldehyde for 2-methoxybenzaldehyde.

### Acid-B: 3-(2-cyanophenyl)-5-methylisoxazole-4-carboxylic acid

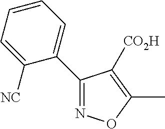

¹H-NMR (DMSO, 400 MHz): δ 7.83-7.98 (m 1H)), 7.74-7.8 (m, 1H), 7.5-7.7 (m, 2H), 2.74 (3H, s). m/e 228.90 (M+1)⁺.

### Acid-C: 3-(2-(benzyloxy)phenyl)-5-methylisoxazole-4-carboxylic acid

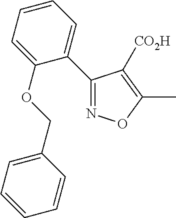

¹H-NMR (DMSO, 400 MHz): δ 11.5 (s, 1H), 9.4 (S, 1H), 9.1 (S, 1H), 8.4 (m, 1H), 8.1-8.15 (dd 1H)), 7.8-7.85 (dd, 1H), 7.5-7.7 (m, 3H), 3.5 (2H, bs), 2.5 (3H, s). m/e 310.09 (M+1)⁺.

### Acid-D: 3-(2-ethylphenyl)-5-methylisoxazole-4-carboxylic acid

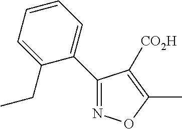

¹H-NMR (DMSO, 400 MHz): δ 7.16-7.44 (4H, m), 3.15-3.51 (q, 2H), 2.67-2.89 (3H, s), 1.03-1.24 (3H, t). m/e 231.84 (M+1)⁺.

### Acid-E: 5-methyl-3-(2-(trifluoromethyl)phenyl)isoxazole-4-carboxylic acid

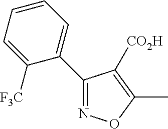

¹H-NMR (DMSO, 400 MHz): δ 7.83-8.05 (1H, m), 7.64-7.81 (2H, m), 7.42-7.64 (1H, s), 2.73 (3H, t). m/e 271.14 (M+1)⁺.

### Acid-F: 3-(2-(difluoromethoxy)phenyl)-5-methylisoxazole-4-carboxylic acid

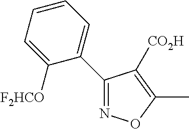

¹H-NMR (DMSO, 400 MHz): δ 13.31 (1H, s), 7.51-7.69 (1H, m), 7.41-7.50 (1H, m), 7.18-7.40 (1H, m), 7.02-7.18 (1H, m), 6.8-6.96 (1H, s), 2.73 (3H, t). m/e 270.02 (M+1)⁺.

### Acid-G: 3-(3-fluoro-2-methylphenyl)-5-methylisoxazole-4-carboxylic acid

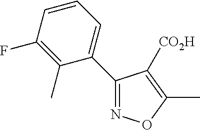

¹H-NMR (DMSO, 400 MHz): δ 7.21-7.42 (2H, m), 7.03-7.2 (1H, m), 2.77 (3H, s).

### Acid-H: 3-(2-chloro-6-fluorophenyl)-5-methylisoxazole-4-carboxylic acid

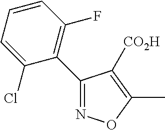

¹H-NMR (DMSO, 400 MHz): δ 7.30-7.68 (3H, m), 2.68 (3H, s). m/e 255.98 (M+1)⁺.

### Acid-I: 3-(2,3-dimethoxyphenyl)-5-methylisoxazole-4-carboxylic acid

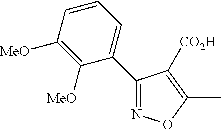

¹H-NMR (DMSO, 400 MHz): δ 7.1-7.2 (2H, m), 6.9 (1H, m), 3.87 (3H, m), 3.58 (3H, m), 2.65 (3H, s).

### Acid-J: 3-(4-fluoro-2-methoxyphenyl)-5-methylisoxazole-4-carboxylic acid

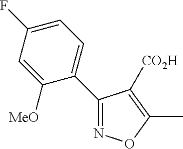

¹H-NMR (CD₃OD, 400 MHz): δ 7.36-7.29 (1H, m), 6.90-6.84 (1H, m), 6.80-6.72 (1H, m), 3.77 (3H, s), 2.68 (3H, s).

### Acid-K: 3-(2-chloro-4-fluorophenyl)-5-methylisoxazole-4-carboxylic acid

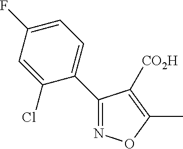

¹H-NMR (CD₃OD, 400 MHz): δ 7.48-7.41 (1H, m), 7.38-7.31 (1H, m), 7.23-7.14 (1H, m), 2.74 (3H, s).

### Acid-L: 3-(2,5-dichlorophenyl)-5-methylisoxazole-4-carboxylic acid

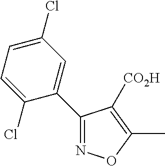

¹H-NMR (CD₃OD, 400 MHz): δ 7.51-7.48 (2H, m), 7.46-7.42 (1H, m), 2.75 (3H, s).

### Acid-M: 3-(5-bromo-2-methoxyphenyl)-5-methylisoxazole-4-carboxylic acid

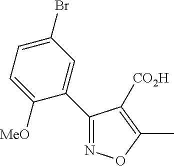

¹H-NMR (DMSO, 400 MHz): δ 12.82 (1H, s), 7.65 (1H, dd, J1=8.8 Hz, J2=2.5 Hz), 7.45 (1H, d, J=2.5 Hz), 7.10 (1H, d, J=8.8 Hz), 3.72 (3H, s), 2.66 (3H, s).

¹H-NMR (CDCl₃, 500 MHz): δ 7.0-7.1 (1H, m), 6.9 (1H, m), 6.52 (1H, m), 2.65 (3H, s).

### Acid-N: 5-methyl-3-(quinolin-8-yl)isoxazole-4-carboxylic acid

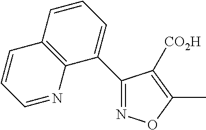

¹H-NMR (DMSO, 400 MHz): δ 8.85 (1H, s), 8.17 (1H, dd, J1=7.5 Hz, J2=1.5 Hz), 8.14 (1H, dd, J1=7.5 Hz, J2=1.5 Hz), 7.82 (1H, m), 7.69 (1H, s), 7.58 (1H, m), 2.73 (3H, s).

### Acid-O: 5-methyl-3-(pyridin-2-yl)isoxazole-4-carboxylic acid

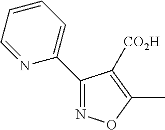

¹H-NMR (DMSO, 400 MHz): δ 15.68-16.07 (1H, s), 8.73 (2H, m), 8.14 (1H, m), 7.64 (1H, m), 2.73 (3H, s). LCMS 205.2

### Acid-P: 5-methyl-3-(pyrimidin-5-yl)isoxazole-4-carboxylic acid

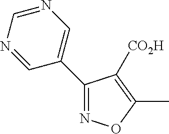

¹H-NMR (DMSO, 400 MHz): δ 9.23-9.37 (1H, m), 8.96-9.22 (2H, m), 2.73 (3H, s).

### Acid-Q: 5-methyl-3-(3-methylpyridin-2-yl)isoxazole-4-carboxylic acid

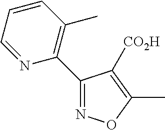

¹H-NMR (DMSO, 400 MHz): δ 8.45 (1H, m), 7.86 (1H, m), 7.46 (1H, m), 2.72 (3H, s), 2.16 (3H, s).

### Acid-R: 3′,5,5′-trimethyl-3,4′-biisoxazole-4-carboxylic acid

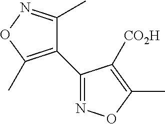

¹H-NMR (DMSO, 400 MHz): δ 2.57 (3H, s), 2.41 (3H, s), 2.27 (3H, s).

### Acid-S: 5-methyl-3-(3-methylthiophen-2-yl)isoxazole-4-carboxylic acid

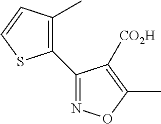

¹H-NMR (DMSO, 400 MHz): δ 7.49-7.74 (dd, 1H), 6.96-7.24 (dd, 1H), 2.7 (s, 3H), 2.13 (s, 3H). m/e 224 (M+1)⁺.

### Acid-T: 3-(2-methoxypyridin-3-yl)-5-methylisoxazole-4-carboxylic acid

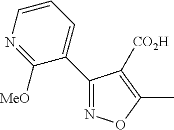

¹H-NMR (DMSO, 400 MHz): δ 8.39-8.59 (m, 1H), 7.64-7.89 (m, 1H), 6.96-7.11 (m, 1H), 3.89 (3H, S), 2.89 (s, 3H).

### Acid-U: 3-(2,3-dihydrobenzofuran-7-yl)-5-methylisoxazole-4-carboxylic acid

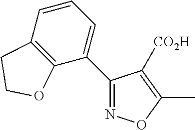

¹H-NMR (DMSO, 400 MHz): δ 7.2-7.38 (1H, m), 7.0-7.20 (1H, m), 6.75-6.94 (1H, m), 4.4-4.58 (2H), 3.09-3.29 (2H), 3.09-3.28 (2H), 2.69 (3H, s). m/e 246.04 (M+1)⁺.

### Acid-V: 3-(benzo[d][1,3]dioxol-4-yl)-5-methylisoxazole-4-carboxylic acid

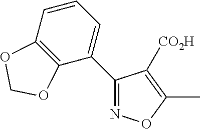

¹H-NMR (DMSO, 400 MHz): δ 7.1-7.2 (1H, m), 6.9 (2H, m), 6.09 (2H, m), 2.62 (3H).

### Acid-W: 3-(2-ethoxypyridin-3-yl)-5-methylisoxazole-4-carboxylic acid

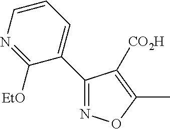

¹H-NMR (DMSO, 400 MHz): δ 8.21-8.59 (1H, m), 7.54-7.89 (1H, m), 6.96-7.11 (1H, m), 4.14-4.36 (q, 2H), 3.89 (3H, S), 2.89 (s, 3H), 1.07-1.26 (3H, t). m/e 249.06 (M+1)⁺.

### Acid-X: 5-methyl-3-(naphthalen-1-yl)isoxazole-4-carboxylic acid

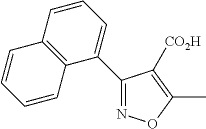

¹H-NMR (DMSO, 400 MHz): δ 7.83-8.22 (m, 3H), 7.4-7.7 (m, 4H), 3.5 (2H, bs), 2.74 (3H, s). m/e 253.97 (M+1)⁺.

### Acid-Y: 5-methyl-3-(quinolin-5-yl)isoxazole-4-carboxylic acid

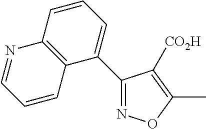

¹H-NMR (DMSO, 400 MHz): δ 8.97 (1H, s), 8.12-8.31 (2H, m), 7.83-8.11 (1H, m), 7.64-7.82 (1H, m), 7.51-7.63 (1H, m), 2.80 (3H, s). m/e 255.5 (M+1)⁺.

### Acid-Z: 3-(isoquinolin-5-yl)-5-methylisoxazole-4-carboxylic acid

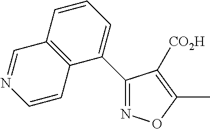

¹H-NMR (DMSO, 400 MHz): δ 9.14-9.49 (1H, m), 8.37-8.47 (1H, m), 8.24-8.37 (1H, m), 7.76-7.98 (2H, m), 7.52-7.72 (1H, m), 2.82 (3H, s). m/e 254.80 (M+1)⁺.

### Acid-AA: 5-methyl-3-(quinolin-4-yl)isoxazole-4-carboxylic acid

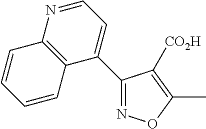

¹H-NMR (DMSO, 400 MHz): δ 12.79-13.30 (1H, s), 8.84-9.12 (1H, m), 8.01-8.21 (1H, m), 7.73-7.95 (1H, m), 7.46-7.72 (2H, m), 2.63 (3H, s). m/e 255.06 (M+1)⁺.

### Acid-AB: 5-methyl-3-(1-methyl-1H-benzo[d]imidazol-2-yl)isoxazole-4-carboxylic acid

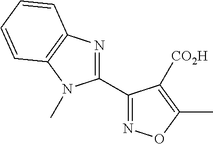

¹H-NMR (DMSO, 400 MHz): δ 7.86-7.89 (2H, m), 7.31-7.86 (2H, m), 3.97 (3H, s), 2.78 (3H, s). m/e 258.04 (M+1)⁺.

### Acid-AC: 3-(2,3-dihydrobenzo[b][1,4]dioxin-5-yl)-5-methylisoxazole-4-carboxylic acid

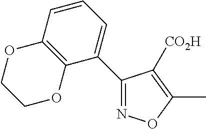

¹H-NMR (DMSO, 400 MHz): δ 6.72-7.08 (3H, m), 4.2-4.36 (4H, m), 2.68 (3H, s). m/e 262.01

### Acid-AD: 3-(2,2-dimethyl-2,3-dihydrobenzofuran-7-yl)-5-methylisoxazole-4-carboxylic acid

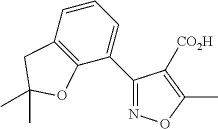

¹H-NMR (DMSO, 400 MHz): δ 7.21-7.34 (1H, m), 7.09-7.2 (1H, m), 6.79-6.96 (1H, m), 2.94-3.11 (2H, m), 2.68 (3H, s), 1.38-1.53 (3H, m), 1.24-1.38 (3H, m). m/e 274.03 (M+1)⁺.

### Acid-AE: 5-methyl-3-(2-(pyridin-3-yl)phenyl)isoxazole-4-carboxylic acid

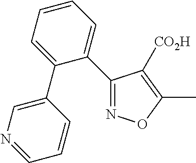

¹H-NMR (DMSO, 400 MHz): δ 8.88-9.05 (1H, m), 8.54-8.78 (1H, m), 7.43-8.40 (3H, m), 7.4-7.71 (3H, m), 2.47 (3H, s). m/e 281.04 (M+1)⁺.

### Acid-AF: 5-methyl-3-(3-(pyridin-3-yl)phenyl)isoxazole-4-carboxylic acid

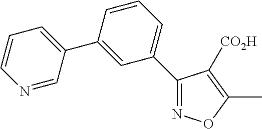

¹H-NMR (DMSO, 400 MHz): δ 8.41-8.58 (1H, m), 8.3 (1H, s), 7.43-7.77 (5H, m), 7.14-7.40 (1H, m), 2.94-3.11 (1H, m), 2.47 (3H, s). m/e 281.04 (M+1)⁺.

### Acid-AG: 3-(3-chloropyridin-4-yl)-5-methylisoxazole-4-carboxylic acid

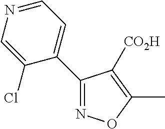

¹H-NMR (CD₃OD, 300 MHz): δ 8.63 (1H, s), 8.53 (1H, d, J=4.8 Hz), 7.49 (1H, d, J=4.8 Hz), 2.75 (3H, s).

### Acid-AH: 3-(2-chloropyridin-3-yl)-5-methylisoxazole-4-carboxylic acid

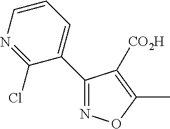

¹H-NMR (CD₃OD, 500 MHz): δ 8.52-8.44 (1H, m), 7.92-7.83 (1H, m), 7.53-7.44 (1H, m), 2.76 (3H, s).

### Acid-AI: 5-methyl-3-(quinoxalin-5-yl)isoxazole-4-carboxylic acid

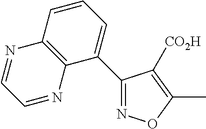

¹H-NMR (DMSO, 500 MHz): δ 12.62 (1H, s), 9.01 (1H, d, J=1.8 Hz), 8.91 (1H, d, J=1.5 Hz), 8.28-8.21 (1H, m), 7.99-7.92 (2H, m), 2.74 (3H, s).

### Acid-AJ: 3-(2-methoxynaphthalen-1-yl)-5-methylisoxazole-4-carboxylic acid

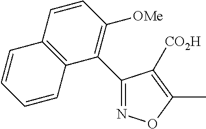

¹H-NMR (DMSO, 400 MHz): δ 12.60 (1H, s), 8.09 (1H, d, J=9.0 Hz), 7.93 (1H, d, J=7.8 Hz), 7.55 (1H, d, J=9.0 Hz), 7.46-7.33 (3H, m), 3.84 (3H, s), 2.77 (3H, s).

### Acid-AK: 5-(fluoromethyl)-3-(2-methoxyphenyl)isoxazole-4-carboxylic acid

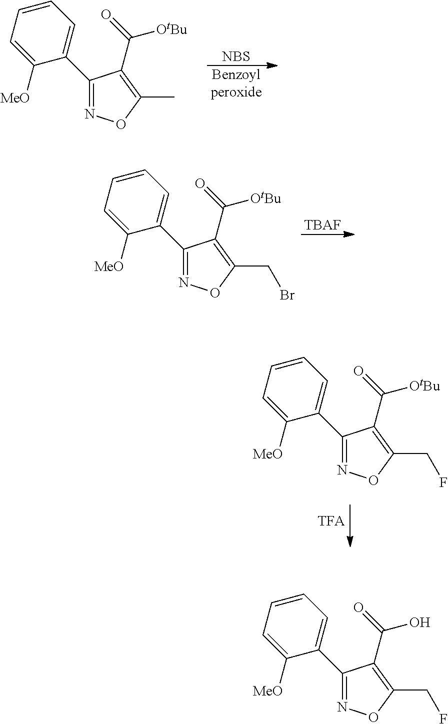

### Step AK1

A solution of tert-butyl 3-(2-methoxyphenyl)-5-methylisoxazole-4-carboxylate (0.795 g, 2.75 mmol), NBS (0.978 g, 5.50 mmol) and benzoyl peroxide (0.033 g, 0.137 mmol) in CCl₄ (10 mL) was stirred at 90° C. for 16 hours. The reaction mixture was cooled to RT and the solid was filtered off. Solvent was removed in vacuo. The product was purified by flash chromatography (DCM, Rf 0.56) to give 0.44 g (44% yield). ¹H-NMR (CDCl₃, 400 MHz): δ 7.49-7.37 (2H, m), 7.04 (1H, t, J=7.5 Hz), 6.96 (1H, d, J=8.3 Hz), 4.79 (2H, s), 3.79 (3H, s), 1.38 (9H, s).

### Step AK2

tert-butyl 5-(fluoromethyl)-3-(2-methoxyphenyl)isoxazole-4-carboxylate was prepared from tert-Butyl 5-(bromomethyl)-3-(2-methoxyphenyl)isoxazole-4-carboxylate by the method described in Sun, H.; DiMagno, S. G., J. Am. Chem. Soc. 2005, 127, 2050-2051. ¹H-NMR (CDCl₃, 400 MHz): δ 7.49-7.37 (2H, m), 7.08-7.01 (1H, m), 6.97 (1H, d, J=8.3 Hz), 5.72 (2H, d, J=47.2 Hz), 3.79 (3H, s), 1.35 (9H, s).

### Step AK3

Treating tert-butyl 5-(fluoromethyl)-3-(2-methoxyphenyl)isoxazole-4-carboxylate with TFA/DCM (1:1) at room temperature for one hour followed by evaporation of DCM/TFA in vacuo provided the title compound. ¹H-NMR (CD₃OD, 400 MHz): δ 7.53-7.44 (1H, m), 7.42-7.35 (1H, m), 7.13-6.98 (2H, m), 5.74 (2H, d, J=47.2 Hz), 3.78 (3H, s).

### Acid-AL: 5-(difluoromethyl)-3-(2-chlorophenyl)isoxazole-4-carboxylic acid

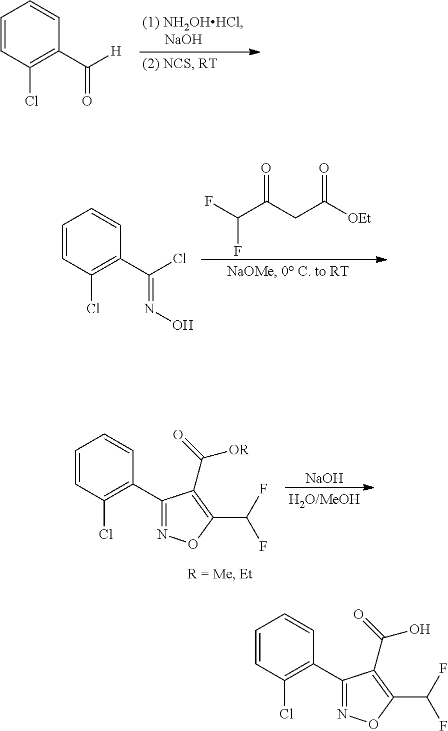

### Step AL1

A mixture of methyl 3-(2-chlorophenyl)-5-(difluoromethyl)isoxazole-4-carboxylate and ethyl 3-(2-chlorophenyl)-5-(difluoromethyl)isoxazole-4-carboxylate was synthesized as described in [Roy, A. K.; Batra, S. Synthesis 2003, 1347-56.]. Analytical samples of the two esters were obtained by isolation with preparative HPLC. Methyl 3-(2-chlorophenyl)-5-(difluoromethyl)isoxazole-4-carboxylate. ¹H NMR (CDCl₃, 300 MHz) δ 7.36-7.56 (4H, m), 7.25 (1H, t, J=52.3 Hz), 3.78 (3H, s).

Ethyl 3-(2-chlorophenyl)-5-(difluoromethyl)isoxazole-4-carboxylate. ¹H NMR (CDCl₃, 300 MHz) δ 7.36-7.54 (4H, m), 7.27 (1H, t, J=52.3 Hz), 4.22 (2H, q, J=7.0 Hz), 1.13 (3H, t, J=7.3 Hz)

### Step AL2

Hydrolysis of the mixture of the two esters obtained from Step AL1 following the procedure described in Step A1 of Acid-A provided the title compound. ¹H-NMR (CD₃OD, 300 MHz): δ 7.55-7.46 (2H, m), 7.45-7.36 (2H, m), 6.73 (1H, t, J=54.1 Hz).

### Acid-AM: 3-(2-chlorophenyl)-5-methyl-1H-pyrazole-4-carboxylic acid

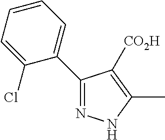

The title compound was prepared according to the literature procedure: El Kaim, L.; Lacroix, S. Synlett, 2000, 3, 353-354. ¹H-NMR (CDCl₃, 400 MHz): δ 7.45-7.55 (1H, m), 7.28-7.40 (2H, m), 7.25-7.27 (1H, m), 2.44 (3H, s).

### Acid-AN: 5-(2-chlorophenyl)-1,3-dimethyl-1H-pyrazole-4-carboxylic acid

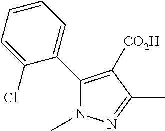

The title compound was prepared by analogy to Acid-AM, substituting methyl hydrazine for hydrazine and separating the regioisomers.

¹H-NMR (CDCl₃, 400 MHz): δ 7.33-7.35 (1H, m), 7.29-7.30 (1H, m), 7.25-7.27 (1H, m), 3.59 (3H, m), 2.52 (3H, s).

### Acid-AO: 2-(2-Chlorophenyl)-4-methyl-1H-pyrrole-3-carboxylic acid

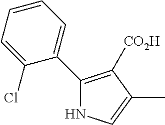

The title compound was prepared according to the literature procedure: Grigg, R.; Savic, V. Chem. Commun. 2000, (10), 873-874. ¹H-NMR (CDCl₃, 400 MHz): δ 7.52-7.63 (1H, m), 7.41-7.48 (2H, m), 7.28-7.32 (1H, m), 6.8-6.85 (1H, m), 2.17 (3H, s).

### Acid-AP: 3-(2,6-dichlorophenyl)-5-methyl-4,5-dihydroisoxazole-4-carboxylic acid

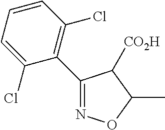

The title compound was prepared as described in: Golebiewski, W. M.; Gucma, M. Journal of Heterocyclic Chemistry (2008), 45(6), 1687-1693.

### Acid-AQ: 1-(2-chlorophenyl)-4-methyl-1H-pyrazole-5-carboxylic acid

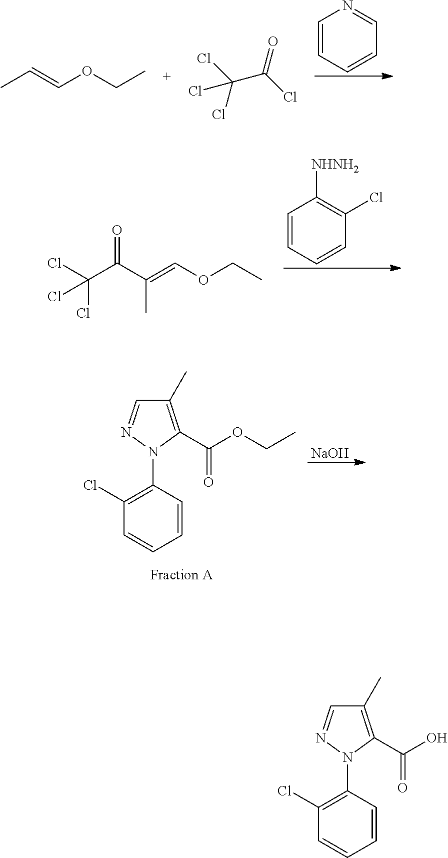

Reference: Martins, M. A. P. et al. J. Molecular Catalysis A: Chemical, 2007, 266, 100.

### Step AQ1

A mixture of (E)-1-ethoxyprop-1-ene (6.41 mL, 57.9 mmol) and pyridine (4.68 mL, 57.9 mmol) was added to a soln. of 2,2,2-trichloroacetyl chloride (10.53 g, 57.9 mmol) in DCM (15 mL) at −10° C. at a rate of 6-10 drops/min. After the addition was completed, the mixture was stirred at r.t for 24 hrs. Filtered, and the filtrate was concentrated in reduced pressure (at first the temperature of the water bath was r.t, after most DCM was evaporated off, water bath was warmed to 50° C. to afford (E)-1,1,1-trichloro-4-ethoxy-3-methylbut-3-en-2-one (2.55 g, 10.79 mmol, 18.64% yield). 1H-NMR (500 MHz, CDCl₃): δ: 7.96 (s, 1H), 4.21 (q, J=7.1 Hz, 2H), 1.94 (s, 3H), 1.41 (t, J=7.2 Hz, 3H). This material was taken into Step G2 without further purification.

### Step AQ2

A mixture of (E)-1,1,1-trichloro-4-ethoxy-3-methylbut-3-en-2-one (279 mg, 1.205 mmol) and (2-chlorophenyl)hydrazine, HCl (253 mg, 1.446 mmol) in EtOH (5 mL) was heated to reflux for 3 hrs. Cooled to r.t. then separated by prep-HPLC to afford ethyl 1-(2-chlorophenyl)-4-methyl-1H-pyrazole-5-carboxylate (Fraction A, 30 mg, 0.113 mmol, 9.37% yield).

### Step AQ3

A solution of ethyl 1-(2-chlorophenyl)-4-methyl-1H-pyrazole-5-carboxylate (120 mg, 0.453 mmol) in a 1:1 mixture of Sodium hydroxide (2 mL, 6.00 mmol) and Methanol (2 mL) was stirred at rt for 2 h. Concentrated to remove the solvent. The residue was taken up in EtOAc and water. The aqueous layer was acidified with 6M HCl to PH=˜3, extracted with EtOAc (3×). The combined organic layer was dried (Na₂SO₄) and concentrated to afford 50 mg (47%) of 1-(2-chlorophenyl)-4-methyl-1H-pyrazole-5-carboxylic acid. ¹H-NMR (CDCl₃, 400 MHz): δ 7.78-7.92 (1H, m), 7.56-7.71 (2H, m), 7.44-7.55 (2H, m), 2.38 (3H, s).

### Acid-AR: 5-(2-methoxyphenyl)-3-methylisoxazole-4-carboxylic acid

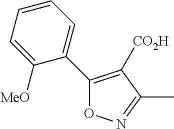

The title compound was prepared as described in: Chantegrel, B.; Nadi, A. I.; Gelin, S. J. Org. Chem, 1984, 49, 4419-4424.

¹H-NMR (CDCl₃, 400 MHz): δ 7.36-7.55 (2H, m), 6.86-7.25 (2H, m), 2.50 (3H, s).

### Acid-AS: 5-(2-chlorophenyl)-3-methylisoxazole-4-carboxylic acid

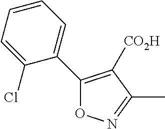

The title compound was prepared by analogy to Acid-AR.

¹H-NMR (CDCl₃, 400 MHz): δ 7.41-7.69 (2H, m), 2.44 (3H, s). m/e 238.02 (M+1)⁺.

### Acid-AT: 5-(2-methoxyphenyl)-3-methyl-1H-pyrazole-4-carboxylic acid

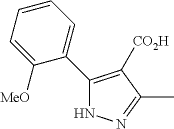

The title compound was prepared according to the literature procedure: El Kaim, L.; Lacroix, S. Synlett, 2000, 3, 353-354. ¹H-NMR (CDCl₃, 400 MHz): δ 7.28-7.40 (1H, m), 7.17-7.25 (1H, m), 6.90-7.08 (2H, m), 2.38 (3H, s). m/e 233.23 (M+1)⁺.

### Acid-AU: 4-(2-methoxyphenyl)-1-methyl-1H-1,2,3-triazole-5-carboxylic acid

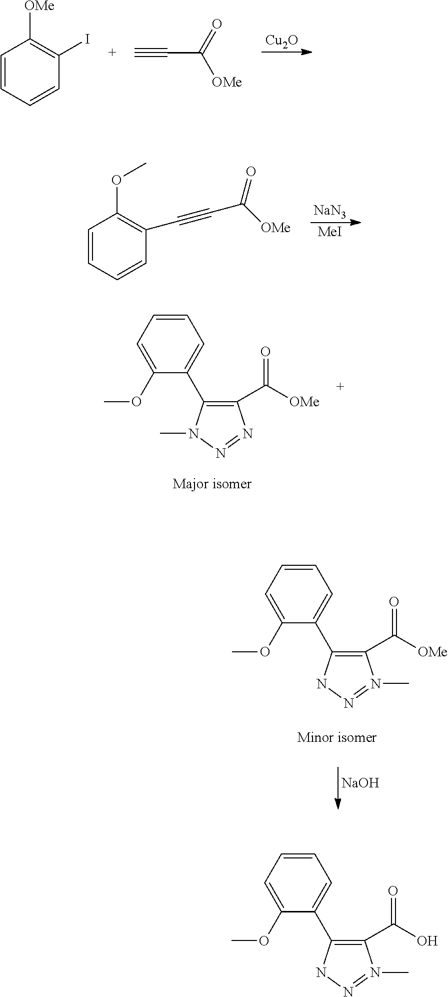

### Step AU1

Reference: Liliebris, C.; Larsen, S. D; Ogg, D.; Palazuk, B. J; and Pleasdale, J. E. J. Med. Chem., 2002, 45, 1785.

To a suspension of methyl propiolate (2, 1.314 ml, 15.41 mmol) and copper(I) oxide (1.086 g, 7.59 mmol) in DMF (20 ml) was added 1-iodo-2-methoxybenzene (1, 1.262 ml, 9.48 mmol). The resulting mixture was heated in a Microwave reactor to 110° C. for 2 hrs. The reaction mixture was filtered through a short pad of silica gel and washed w/EtOAc. The organic layer was washed w/1M HCl, brine and sat'd NaHCO₃, dried (Na₂SO₄) and concentrated to leave an oil as crude product, which was purified by flash chromatography (SiO₂, EtOAc/Hexane, 1:2) to afford methyl 3-(2-methoxyphenyl)propiolate 3 (932 mg, 4.66 mmol, 49.1% yield). ¹H-NMR (500 MHz, CDCl₃), δ: 7.54 (1H, dd), 7.44 (1H, td), 6.98-6.92 (m, 2H), 3.92 (s, 3H), 3.86 (s, 3H).

### Step AU2

A mixture of methyl 3-(2-methoxyphenyl)propiolate (3, 932 mg, 4.90 mmol), sodium azide (478 mg, 7.35 mmol), and iodomethane (0.458 ml, 7.35 mmol) in Water (7 ml) and DMF(3 ml) was heated to 100° C. in a Microwave reactor for 6 hrs. Purified by Prep-HPLC (Varian, 15-90B in min., B=90% MeOH/10% H2O) to afford methyl 5-(2-methoxyphenyl)-1-methyl-1H-1,2,3-triazole-4-carboxylate (701 mg, 2.69 mmol, 55.0% yield), ¹H-NMR (500 MHz, CD3OD), δ: 7.52-7.56 (m, 1H), 7.30 (dd, 1H), 7.12, (td, 1H), 7.06, (d, 1H), 3.90 (s, 3H), 3.86 (s, 3H), 3.82 (s, 3H). and methyl 4-(2-methoxyphenyl)-1-methyl-1H-1,2,3-triazole-5-carboxylate (185 mg, 0.711 mmol, 14.51% yield), ¹H-NMR (500 MHz, CD3OD), δ: 7.49-7.46 (m, 2H), 7.10-7.07 (m, 2H), 4.32 (s, 3H), 3.79 (s, 3H), 3.78 (s, 3H).

### Step AU3

To a soln. of methyl 4-(2-methoxyphenyl)-1-methyl-1H-1,2,3-triazole-5-carboxylate (183 mg, 0.74 mmol) in MeOH (5 ml) was added sodium hydroxide (1.3 ml, 3M aq. solution). The mixture was stirred at r.t for 2 hrs, then concentrated in vacuo. The residue was taken up in water, washed w/ether (3×) to remove the possible impurity. The aq. phase was acidified w/6M HCl to pH 3, extracted w/EtOAc (4×). The combined organic layer was dried and evaporated to afford the title compound (164 mg, 0.703 mmol, 95% yield). ¹H-NMR (500 MHz, CD3OD), δ: 7.46-7.42 (m, 2H), 7.09-7.05 (m, 2H), 4.32 (s, 3H), 3.73 (s, 3H).

### Acid-AV: 4-(2-methoxyphenyl)-1-methyl-1H-imidazole-5-carboxylic acid

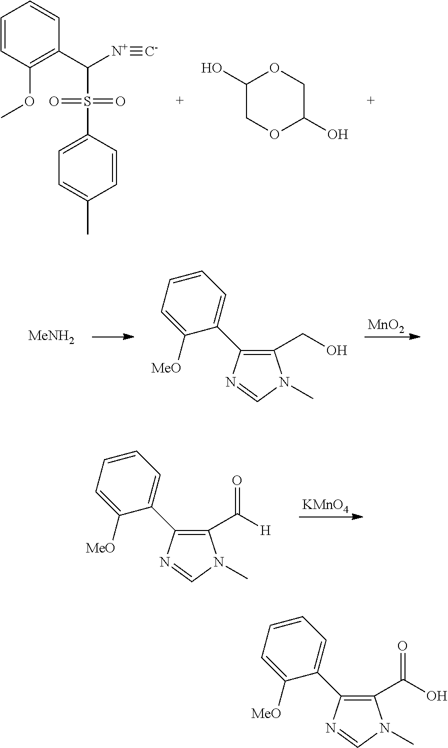

Reference: Luke, R. W. A.; Jones, C. D.; McCoull, W; Hayter, B. R. WO Patent 2004013141, 2004.

### Step AV1

To a soln. of 1,4-dioxane-2,5-diol (120 mg, 0.995 mmol) in THF (8 ml) was added methylamine (2.8 ml, 0.664 mmol) at r.t. The resulting mixture was stirred at r.t for 75 min. Then 1-(isocyano(tosyl)methyl)-2-methoxybenzene (200 mg, 0.664 mmol) was added while keeping reaction mixture at <30° C. by a water bath. The reaction mixture was stirred at r.t overnight. Evaporated to leave white solid, dissolved in DMF, and purified by Pre-HPLC to afford (4-(2-methoxyphenyl)-1-methyl-1H-imidazol-5-yl)methanol (84 mg, 0.377 mmol, 38.6% yield) as a colorless oil. ¹H-NMR (MeOD), δ: 8.97 (1H, s), 7.55 (1H, t, J=7.5 Hz), 7.47 (1H, d, J=8.0 Hz), 7.22 (1H, d, 8.0 Hz), 7.15 (1H, t, J=7.5 Hz), 4.67 (2H, s), 4.05 (3H, s), 3.89 (3H, s).

### Step AV2

To a soln. of (4-(2-methoxyphenyl)-1-methyl-1H-imidazol-5-yl)methanol (84 mg, 0.385 mmol) in 1,4-Dioxane (5 ml) was added MnO2 (147 mg, 1.693 mmol). The mixture was heated to 90° C. for 4 hrs or until LC/MS showed the completion of the reaction. The reaction mixture was Filtered through celite, and evaporated to afford 4-(2-methoxyphenyl)-1-methyl-¹H-imidazole-5-carbaldehyde (78 mg, 0.325 mmol, 84% yield), which was used for the next reaction w/o purification.

### Step AV3

To a soln. of 4-(2-methoxyphenyl)-1-methyl-1H-imidazole-5-carbaldehyde (78 mg, 0.361 mmol) in acetone (5 ml) and water (1 ml) was added potassium carbonate (100 mg, 0.721 mmol). After potassium was dissolved, KMnO4 (123 mg, 0.776 mmol) was added at r.t. The mixture was stirred for 24 hrs. LC/MS showed the completion. The mixture was filtered through celite, washed w/water. The actone was evaporated from the filtrate which was extracted with EtOAc (2×). The aq. layer was acidified w/HOAc to PH=5, reduced the volume to half volume, freezed and lyphilized to leave solid, which was purified by Pre-HPLC to afford the title compound (41 mg, 0.173 mmol, 48% yield). ¹H-NMR (MeOD), δ: 8.06 (s, 1H), 7.41-7.36 (m, 2H), 7.05-6.99 (m, 2H), 3.98 (s, 3H), 3.80 (s, 3H).

### Acid-AW: 3-(4-Methoxyphenyl)-5-methylisothiazole-4-carboxylic acid

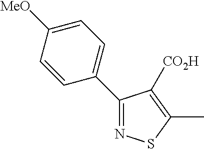

The title compound was prepared as described in [Gerritz, S.; Shi, S.; Zhu, S. U.S. Pat. Appl. 2006, US 2006287287.]

### Acid-AX: 1-(2-chlorophenyl)-4-methyl-1H-1,2,3-triazole-5-carboxylic acid

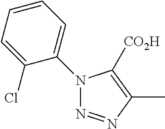

Methyl 1-(2-chlorophenyl)-4-methyl-¹H-1,2,3-triazole-5-carboxylate (methyl ester of Acid-AX) was prepared as described in [Bell, M. G. et al. PCT Int. Appl. 2007, WO 2007140174.] The methyl ester was hydrolyzed (NaOH/H2O/MeOH, RT) to afford the title compound. NMR for methyl ester of Acid-AX: ¹H-NMR (CD₃OD, 400 MHz): δ 7.67-7.58 (2H, m), 7.55-7.51 (2H, m), 3.77 (3H, s), 2.61 (3H, s).

NMR for Acid-AX: ¹H-NMR (CD₃OD, 400 MHz): δ 7.65-7.50 (4H, m), 2.61 (3H, s).

### Acid-AY: 2-(2-methoxyphenyl)-4-methyl-1H-pyrrole-3-carboxylic acid

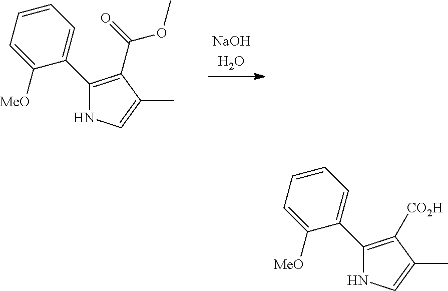

The title compound was prepared by analogy to Acid-AO.

NMR data for Acid-AY methyl ester: ¹H-NMR (CDCl₃, 400 MHz): δ 8.59 (1H, s), 7.42-7.27 (2H, m), 7.02-6.95 (2H, m), 6.58 (1H, s), 3.80 (3H, s), 3.67 (3H, s), 2.32 (3H, s).

A mixture of methyl 2-(2-methoxyphenyl)-4-methyl-¹H-pyrrole-3-carboxylate (0.126 g, 0.514 mmol) and sodium hydroxide (0.205 g, 5.14 mmol) in NMP (4.500 mL) and Water (1.500 mL) was stirred at 120° C. overnight. The crude product was used in amide formation without further purification. LCMS showed both MH⁺ (232.05) and [M−H]⁻ (230.24).

### Acid-AZ: 4-methyl-2-(naphthalen-1-yl)-1H-pyrrole-3-carboxylic acid

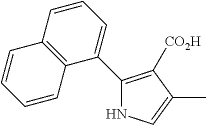

The title compound was prepared by analogy to Acid-AO.

¹H-NMR (CD₃OD, 400 MHz): δ 7.86 (2H, d, J=8.3 Hz), 7.65 (1H, d, J=8.3 Hz), 7.53-7.35 (4H, m), 6.66 (1H, s), 2.35 (3H, s).

### Acid-BA: 3-(2-methoxy-5-nitrophenyl)-5-methylisoxazole-4-carboxylic acid

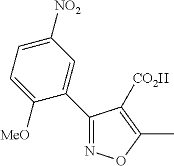

Prepared by analogy to Acid-A.

¹H-NMR (DMSO, 400 MHz): δ 12.94 (1H, s), 8.41 (1H, dd, J1=9.3 Hz, J2=2.9 Hz), 8.17 (1H, d, J=2.9 Hz), 7.38 (1H, d, J=9.3 Hz), 3.88 (3H, s), 2.69 (3H, s).

### Acid-BB: 3-(4-azidophenyl)-5-methylisoxazole-4-carboxylic acid

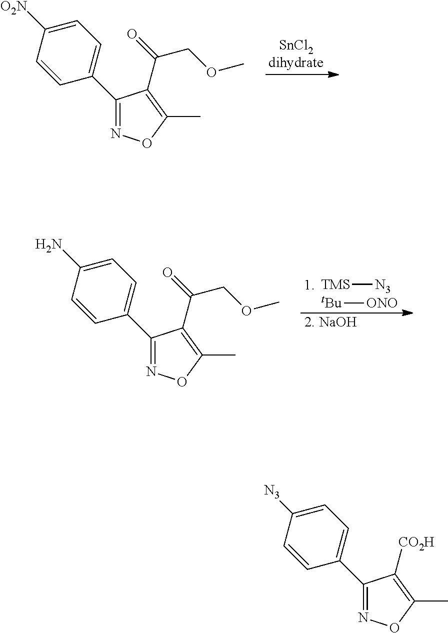

### Step BB1

Ethyl 5-methyl-3-(4-nitrophenyl)isoxazole-4-carboxylate was prepared by analogy to Acid-A. ¹H-NMR (CDCl₃, 500 MHz): δ 8.31 (2H, d, J=8.7 Hz), 7.85 (2H, d, J=8.7 Hz), 4.28 (2H, q, J=7.2 Hz), 2.78 (3H, s), 1.27 (3H, t, J=7.2 Hz).

### Step BB2

Ethyl 3-(4-aminophenyl)-5-methylisoxazole-4-carboxylate was prepared by reducing ethyl 5-methyl-3-(4-nitrophenyl)isoxazole-4-carboxylate with tin(II) chloride dihydrate. ¹H-NMR (CDCl₃, 500 MHz): δ 7.48 (2H, d, J=8.4 Hz), 6.72 (2H, d, J=8.4 Hz), 4.28 (2H, q, J=7.2 Hz), 3.85 (2H, s), 2.71 (3H, s), 1.29 (3H, t, J=7.2 Hz).

### Step BB3

Conversion of ethyl 3-(4-aminophenyl)-5-methylisoxazole-4-carboxylate to ethyl 3-(4-azidophenyl)-5-methylisoxazole-4-carboxylate with the chemistry described in [Barral, K.; Moorhouse, A. D.; Moses, J. E. Org. Lett. 2007, 9, 1809-1811.], followed by the hydrolysis of the ethyl ester provided the title compound. ¹H-NMR (CD₃OD, 500 MHz): δ 7.68 (2H, d, J=8.9 Hz), 7.15 (2H, d, J=8.9 Hz), 2.72 (3H, s).

### Acid BC: 3-(2-(methoxycarbonyl)phenyl)-5-methylisoxazole-4-carboxylic acid

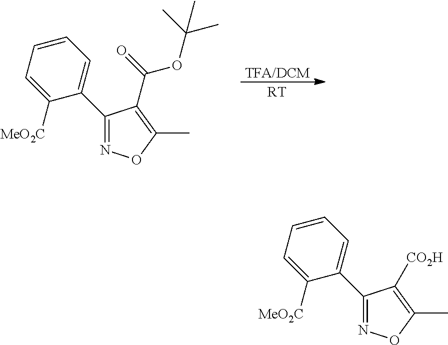

The mixture of tert-butyl 3-(2-(methoxycarbonyl)phenyl)-5-methylisoxazole-4-carboxylate (558 mg, 1.758 mmol) and TFA (4.0 mL, 51.9 mmol) was stirred at RT for one hour. TFA was evaporated in vacuo to give 0.450 g (98%, theoretical yield 0.459 g) of the acid.

¹H-NMR (DMSO, 400 MHz): δ 12.85 (1H, s), 7.99 (1H, dd, J1=7.5 Hz, J2=1.5 Hz), 7.72-7.60 (2H, m), 7.45 (1H, dd, J1=7.5 Hz, J2=1.5 Hz), 3.67 (3H, s), 2.70 (3H, s). ¹³C-NMR (DMSO, 100 MHz) δ 173.9, 166.0, 162.7, 162.5, 132.0, 131.0, 130.5, 129.7, 129.70, 129.6, 109.5, 51.9, 12.8.

### Acid-BD: 3-(5-(tert-butoxycarbonylamino)-2-methoxyphenyl)-5-methylisoxazole-4-carboxylic acid

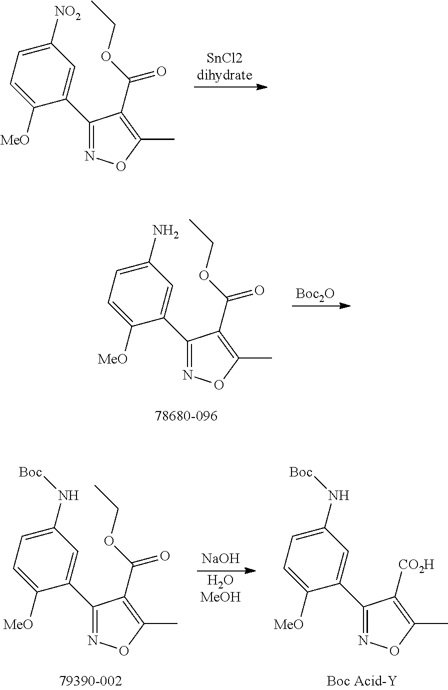

### Step BD1

Ethyl 3-(2-methoxy-5-nitrophenyl)-5-methylisoxazole-4-carboxylate was prepared by analogy to Acid-A.

### Step BD2

Reduction of ethyl 3-(2-methoxy-5-nitrophenyl)-5-methylisoxazole-4-carboxylate (0.54 g, 1.763 mmol) by tin (II) chloride dihydrate (6 eq) in DMF (5 mL) at room temperature in 16 hours provided 0.44 g of ethyl 3-(5-amino-2-methoxyphenyl)-5-methylisoxazole-4-carboxylate. ¹H-NMR (CDCl₃, 400 MHz): δ 6.81-6.76 (3H, m), 4.16 (2H, q, J=7.2 Hz), 3.68 (3H, s), 3.48 (2H, s), 2.70 (3H, s), 1.14 (3H, t, J=7.2 Hz).

### Step BD3

Ethyl 3-(5-amino-2-methoxyphenyl)-5-methylisoxazole-4-carboxylate was Boc-protected under standard conditions. ¹H-NMR (CDCl₃, 400 MHz): δ 7.54 (1H, d, J=8.3 Hz), 7.32 (1H, d, J=2.5 Hz), 6.88 (1H, d, J=8.8 Hz), 6.39 (1H, s), 4.15 (2H, q, J=7.2 Hz), 3.73 (3H, s), 2.70 (3H, s), 1.51 (9H, s), 1.12 (3H, t, J=7.2 Hz).

### Step BD4

Ethyl 3-(5-(tert-butoxycarbonylamino)-2-methoxyphenyl)-5-methylisoxazole-4-carboxylate was hydrolyzed to the title compound by analogy to Step A-2 of Acid-A. ¹H-NMR (CDCl₃, 400 MHz): δ 7.52 (1H, d, J=8.8 Hz), 7.34 (1H, s), 6.91 (1H, d, J=8.8 Hz), 6.43 (1H, s), 3.75 (3H, s), 2.73 (3H, s), 1.52 (9H, s).

### Amine-A: 1-(3-chloro-5-nitropyridin-2-yl)piperazine

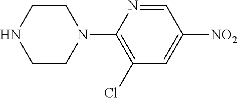

A mixture of piperazine (569 mg, 6.61 mmol) and 2,3-dichloro-5-nitropyridine (255 mg, 1.320 mml) in DMF (5 ml) was stirred at r.t. for 3 hrs. The mixture was poured into water, extracted w/EtOAc (4×). The combined organic layer was dried (Na2SO4) and concentrated to afford 1-(3-chloro-5-nitropyridin-2-yl)piperazine (286 mg, 1.120 mmol, 85% yield). ¹H-NMR (CDCl₃, 400 MHz) δ 8.96 (1H, s), 8.32 (1H, s), 3.70 (4H, m), 3.02 (1H, s), 3.70 (4H, m).

### Amine-B: 1-(3-bromo-5-nitropyridin-2-yl)piperazine

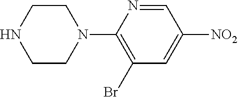

### Step B1

A solution of 3-bromo-2-chloro-5-nitropyridine (0.475 g, 2.000 mmol), tert-butyl piperazine-1-carboxylate (0.373 g, 2 mmol), and triethylamine (0.405 g, 4.00 mmol) in DCM (10 mL) was stirred at RT overnight. Purified by silicon gel column with 5% EtOAc in DCM gave tert-butyl 4-(3-bromo-5-nitropyridin-2-yl)piperazine-1-carboxylate (0.68 g, 1.756 mmol, 88% yield). yellow solid. ¹H-NMR (CDCl₃, 500 MHz) δ 9.02 (1H, s), 8.55 (1H, s), 3.59-3.65 (8H, m), 1.50 (9H, s).

### Step B2

tert-butyl 4-(3-bromo-5-nitropyridin-2-yl)piperazine-1-carboxylate was treated with 30% TFA in DCM (5 ml) for 2 hours. Drained and dried to afford the title compound (0.71 g, 1.770 mmol, 88% yield) as a yellow solid. ¹H-NMR (CD₃OD, 500 MHz) δ 9.08 (1H, s), 8.72 (1H, s), 3.85 (4H, m), 3.41 (4H, m).

### Amine-C: (R)-1-(2-chloro-4-nitrophenyl)-2-methylpiperazine

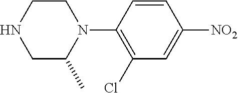

### Step C1

A mixture of (R)-tert-butyl 3-methylpiperazine-1-carboxylate (500 mg, 2.497 mmol), 2-chloro-1-fluoro-4-nitrobenzene (438 mg, 2.497 mmol), and DIEA (436 μL, 2.497 mmol) in a vial was heated at 160-165° C. for 1 h. Purification via silica gel column with DCM gave (R)-tert-butyl 4-(2-chloro-4-nitrophenyl)-3-methylpiperazine-1-carboxylate (478 mg, 1.343 mmol, 53.8% yield). yellow solid. ¹H-NMR (CDCl₃, 500 MHz) δ 8.28 (1H, s), 8.11 (1H, d, J=8.8 Hz), 7.05 (1H, d, J=8.8 Hz), 3.81 (2H, s), 3.56 (2H, s), 3.37 (2H, s), 2.84 (1H, s), 1.50 (9H, s), 1.01 (3H, d, J=6.4).

### Step C2

(R)-tert-butyl 4-(2-chloro-4-nitrophenyl)-3-methylpiperazine-1-carboxylate was stirred with 40% TFA in DCM (5 ml) for 2 h. After drained, dried in vacuum, a brown solid was obtained. 77232-012-02, (R)-1-(2-chloro-4-nitrophenyl)-2-methylpiperazine (516 mg, 1.395 mmol, 55.9% yield). ¹H-NMR (CDCl₃, 500 MHz) δ 8.35 (1H, s), 8.18 (1H, d, J=8.8 Hz), 7.25 (1H, d, J=8.8 Hz), 3.88 (1H, m), 3.56 (2H, m), 3.48-3.57 (4H, m), 3.15 (2H, m), 1.11 (3H, d, J=6.4).

### Amine-D: (S)-1-(2-chloro-4-nitrophenyl)-2-methylpiperazine

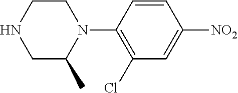

### Step D1

A mixture of (S)-tert-butyl 3-methylpiperazine-1-carboxylate (600 mg, 3.00 mmol), 2-chloro-1-fluoro-4-nitrobenzene (526 mg, 3.00 mmol), and triethylamine (455 mg, 4.49 mmol) was at 160-165° C. for ¹H. Silicon gel column purification with DCM, then 5% EtOAc in DCM gave (S)-tert-butyl 4-(2-chloro-4-nitrophenyl)-3-methylpiperazine-1-carboxylate (683 mg, 1.920 mmol, 64.1% yield), white solid. ¹H-NMR (CDCl₃, 500 MHz) δ 8.24 (1H, s), 8.08 (1H, d, J=8.8 Hz), 7.05 (1H, d, J=8.8 Hz), 3.80 (2H, s), 3.54 (2H, s), 3.37 (2H, s), 2.83 (1H, s), 1.47 (9H, s), 1.00 (3H, d, J=6.4).

### Step D2

(S)-tert-butyl 4-(2-chloro-4-nitrophenyl)-3-methylpiperazine-1-carboxylate was treated with 40% TFA in DCM (5 ml) for 2 h. Drained and concentrated in vacuo gave (S)-1-(2-chloro-4-nitrophenyl)-2-methylpiperazine (722 mg, 1.953 mmol, 65.2% yield), brown solid.). ¹H-NMR (CD₃OD, 500 MHz) δ 8.34 (1H, s), 8.21 (1H, d, J=8.8 Hz), 7.46 (1H, d, J=8.8 Hz), 3.87 (1H, m), 3.51 (2H, m), 3.40 (2H, m), 3.10 (2H, m), 1.09 (3H, d, J=6.4).

### Amine E: (3-(2-chlorophenyl)-5-methylisoxazol-4-yl)(piperazin-1-yl)methanone

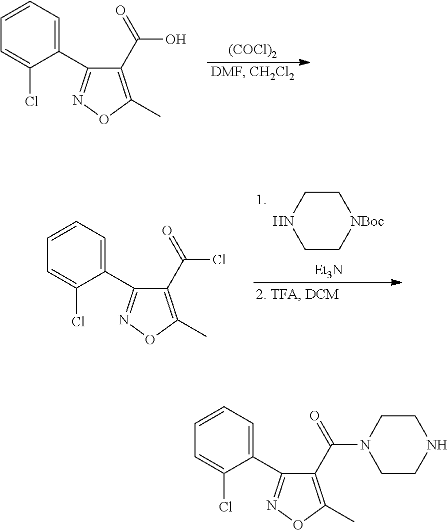

### Steps E1-E2

To a solution of 3-(2-chlorophenyl)-5-methylisoxazole-4-carboxylic acid (2.376 g, 10.00 mmol) in DCM (70 mL) was added 2 drops of DMF, then oxalyl dichloride (1.523 g, 12.00 mmol) in portions. The mixture was stirred for 2 hours and the solution turn clear. The solvent was removed by rotovap and the residue was dried in vacuum for 10 min. The residue was dissolved in DCM (70 mL), then triethylamine (3.04 g, 30.0 mmol) and tert-butyl piperazine-1-carboxylate (1.862 g, 10.00 mmol) was added, the resulting mixture was stirred overnight. After the solvent was removed by rotovap, the residue was purified by silicon gel column with DCM, then 10% EtOAc in DCM to give tert-butyl 4-(3-(2-chlorophenyl)-5-methylisoxazole-4-carbonyl)piperazine-1-carboxylate (3.6 g, 8.87 mmol, 89% yield).

### Step E3

tert-butyl 4-(3-(2-chlorophenyl)-5-methylisoxazole-4-carbonyl)piperazine-1-carboxylate was treated with 50% TFA in DCM (20 ml) for 2 hours. After the solvent was removed, the residue was purified by silicon gel column with 10% MeOH in DCM to give (3-(2-chlorophenyl)-5-methylisoxazol-4-yl)(piperazin-1-yl)methanone (2.4 g, 7.85 mmol, 79% yield). ¹H-NMR (CD₃OD, 500 MHz) δ 7.54-7.61 (3H, m.), 7.50 (1H, t, J=7.3 Hz), 3.63 (4H, s), 2.94 (4H, s), 2.58 (3H, s).

### Amine-G: 1-(2-chloro-4-nitrophenyl)piperazin-2-one

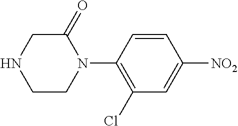

The title compound was prepared from 2-chloro-4-nitroaniline via intramolecular Mitsunobu cyclodehydration as described in [Weissman, S. A. etc. Tetrahedron Lett. 1998, 39, 7459-7462.]

¹H-NMR (CDCl₃, 300 MHz): δ 8.39 (1H, d, J=2.6 Hz), 8.21 (1H, dd, J1=8.8 Hz, J2=2.6 Hz), 7.50 (1H, d, J=8.8 Hz), 3.74 (2H, s), 3.63 (2H, t, J=5.5 Hz), 3.28 (2H, t, J=5.5 Hz), 1.86 (1H, s).

### Amine-I: 1-(5-chloro-3-fluoropyridin-2-yl)piperazine

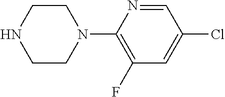

A solution of tert-butyl piperazine-1-carboxylate (745 mg, 4.00 mmol), 5-chloro-2,3-difluoropyridine (598 mg, 4 mmol), and DIEA (699 μL, 4.00 mmol) in NMP was heated at 160-165° C. for 1 h. After cooled down, the residue was treated with 50% TFA in DCM (5 ml) for 1 h. The solvent was removed, then residue was purified by silicon gel column with DCM, then 5% MeOH in DCM to give 1-(5-chloro-3-fluoropyridin-2-yl)piperazine (213 mg, 0.988 mmol, 24.69% yield), white solid. ¹H-NMR (CD₃OD, 500 MHz) δ 8.08 (1H, s), 7.64 (1H, s), 3.69 (4H, m), 3.34 (4H, m).

### Amine-J: (5-chloro-6-(piperazin-1-yl)pyridin-3-yl)methanol

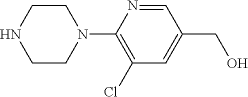

### Step J1

A mixture of tert-butyl piperazine-1-carboxylate (186 mg, 1.000 mmol), (5,6-dichloropyridin-3-yl)methanol (178 mg, 1.000 mmol), and DIEA (175 μL, 1.000 mmol) in NMP was heated at 160-165° C. for 15 min. Silica gel column purification with 25% EtOAc in DCM gave tert-butyl 4-(3-chloro-5-(hydroxymethyl)pyridin-2-yl)piperazine-1-carboxylate (106 mg, 0.323 mmol, 32.3% yield), white solid. ¹H-NMR (CDCl₃, 500 MHz) δ 8.13 (1H, s), 7.66 (1H, s), 4.62 (2H, s), 3.57 (4H, m), 3.28 (4H, m), 1.48 (9H, s).

### Step J2

Tert-butyl 4-(3-chloro-5-(hydroxymethyl)pyridin-2-yl)piperazine-1-carboxylate was stirred with 40% TFA in DCM for 2 h. Drained and dried in vacuum gave the title compound (120 mg, 0.351 mmol, 35.1% yield).

### Amine-K: 1-(5-bromo-3-chloropyridin-2-yl)piperazine

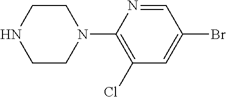

A mixture of tert-butyl piperazine-1-carboxylate (186 mg, 0.996 mmol), 5-bromo-2,3-dichloropyridine (226 mg, 0.996 mmol), and DIEA (174 μL, 0.996 mmol) was heated at 160-165° C. for 1 h. The solid was stirred with 40% TFA in DCM for 2 hours. Purification on silicon gel column with 5% MeOH in DCM gave 1-(5-bromo-3-chloropyridin-2-yl)piperazine (213 mg, 0.770 mmol, 77% yield). ¹H-NMR (CD₃OD, 500 MHz) δ 8.31 (1H, s), 8.01 (1H, s), 3.58 (4H, m), 3.38 (4H, m).

### Amine-L: 1-(3,5-dibromopyridin-2-yl)piperazine

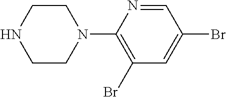

A solution of tert-butyl piperazine-1-carboxylate (279 mg, 1.500 mmol), 3,5-dibromo-2-chloropyridine (407 mg, 1.5 mmol), and DIEA (262 mL, 1.500 mmol) in NMP was heated at 160-165° C. for 1 h. LCMS showed the desired product is the major peak. After cold down, the solid was treated with 40% TFA in DCM for 2 h. Purification in silicon gel column with 5% MeOH/DCM gave 1-(3,5-dibromopyridin-2-yl)piperazine (213 mg, 0.664 mmol, 44.2% yield). ¹H-NMR (CD₃OD, 500 MHz) δ 8.31 (1H, s), 8.01 (1H, s), 3.58 (4H, m), 3.38 (4H, m).

### Amine-M: 1-(3-bromo-5-chloropyridin-2-yl)piperazine

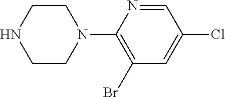

### Step M1

A mixture of tert-butyl piperazine-1-carboxylate (186 mg, 0.996 mmol), 3-bromo-2,5-dichloropyridine (226 mg, 0.996 mmol), and DIEA (174 μL, 0.996 mmol) in NMP was heated at 160-165° C. for 15 min. Silica gel column purification with 5% EtOAc in DCM gave tert-butyl 4-(3-bromo-5-chloropyridin-2-yl)piperazine-1-carboxylate (204 mg, 0.542 mmol, 54.4% yield). White solid. ¹H-NMR (CDCl₃, 500 MHz) δ 8.18 (1H, s), 7.80 (1H, s), 3.58 (4H, m), 3.26 (4H, m), 1.49 (9H, s),

### Step M2

Tert-butyl 4-(3-bromo-5-chloropyridin-2-yl)piperazine-1-carboxylate was stirred with 40% TFA in DCM (5 ml) for 2 h. Drained and dried in vacuum gave 3-bromo-5-chloropyridin-2-yl)piperazine (213 mg, 0.545 mmol, 54.7% yield) as off-white solid. ¹H-NMR (CD₃OD, 500 MHz) δ 8.27 (1H, s), 8.06 (1H, s), 3.54 (4H, m), 3.38 (4H, m).

### Amine-O: 3,5-dichloro-2-(piperazin-1-yl)pyrazine

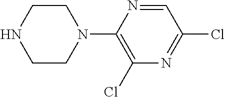

3,5-dichloro-2-(piperazin-1-yl)pyrazine was prepared as described in reference: PCT Int. Appl. 2000, WO 2000076984.

### Example 1

### (3-(2-chlorophenyl)-5-methylisoxazol-4-yl)(4-(2-methyl-4-nitrophenyl)piperazin-1-yl)methanone

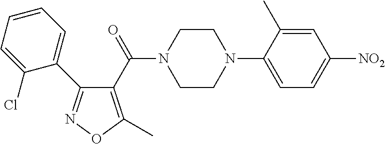

A mixture of 3-(2-chlorophenyl)-5-methylisoxazole-4-carboxylic acid (0.050 g, 0.210 mmol), 1-(2-methyl-4-nitrophenyl)piperazine (0.071 g, 0.210 mmol) (TFA salt), EDC (0.048 g, 0.252 mmol) and DMAP (0.051 g, 0.421 mmol) in dichloromethane (3 mL) was stirred at room temperature overnight. The solvent was evaporated in vacuo. The crude product was purified by preparative HPLC (methanol/water with 0.1% TFA) to give 41 mg (44% yield) of the title compound. ¹H-NMR (300 MHz, CDCl₃) δ 8.08-7.99 (2H, m), 7.60-7.36 (4H, m), 6.81 (1H, d, J=8.4 Hz), 3.79 (2H, br. s.), 3.39 (2H, br. s.), 2.89 (2H, br. s.), 2.61 (3H, s), 2.40 (2H, br. s.), 2.33 (3H, s). HPLC/MS (Method U): (ES+) m/z (M+H)⁺=441; Rₜ=5.67 min.

### Examples 2-5

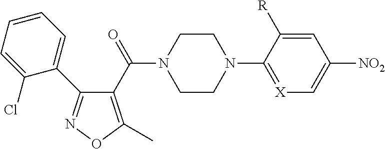

Examples 2-5 were synthesized by analogy to Example 1, substituting the appropriate RHS Preparation for 1-(2-methyl-4-nitrophenyl)piperazine.

| | | | | | LC/MS | RHS |
|---|---|---|---|---|---|---|
| Example | R | X | MH+ | RT | Method | Preparation |
| 2 | CF₃ | CH | 495 | 6.46 | U | Commercial |
| 3 | Cl | N | 462 | 1.54 | K | Amine-A |
| 4 | F | CH | 445 | 2.43 | L | Commercial |
| 5 | Br | N | 508 | 1.60 | M | Amine-B |

### Example 6

### (3-(2-chlorophenyl)-5-methylisoxazol-4-yl)(4-(3-methoxy-5-nitropyridin-2-yl)piperazin-1-yl)methanone

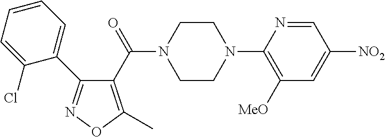

A solution of Amine-E (30 mg, 0.098 mmol), triethylamine (14.89 mg, 0.147 mmol), and 2-chloro-3-methoxy-5-nitropyridine (18.50 mg, 0.098 mmol) in THF (1.5 mL) was stirred at rt overnight. HPLC purification gave the title compound (16.1 mg, 0.035 mmol, 35.5% yield), yellow solid. ¹H-NMR (CD₃OD, 500 MHz) δ 8.64 (1H, s), 7.82 (1H, s), 7.48-7.56 (4H, m), 3.92 (3H, s), 3.70 (4H, s), 3.36 (4H, s), 2.57 (3H, s). HPLC/MS (Method K): (ES+) m/z (M+H)⁺=458; Rₜ=1.47 min.

### Example 7

### (3-(2-chlorophenyl)-5-methylisoxazol-4-yl)(4-(3-methyl-5-nitropyridin-2-yl)piperazin-1-yl)methanone

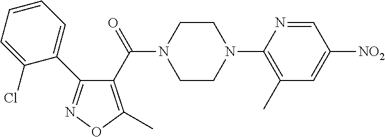

The title compound was synthesized by analogy to Example 6, substituting 2-chloro-3-methyl-5-nitropyridine for 2-chloro-3-methoxy-5-nitropyridine. ¹H-NMR (CDCl₃, 500 MHz) δ 8.93 (1H, s), 8.14 (1H, s), 7.55 (1H, d, J=7.6), 7.51 (1H, d, J=7.9), 7.39-7.46 (2H, m), 3.76 (2H, s), 3.36 (4H, s), 2.96 (2H, s), 2.60 (3H, s), 2.31 (3H, s). HPLC/MS (Method N): (ES+) m/z (M+H)⁺=442; Rₜ=2.18 min.

### Example 8

### (4-(2-bromo-4-nitrophenyl)piperazin-1-yl)(3-(2-chlorophenyl)-5-methylisoxazol-4-yl)methanone

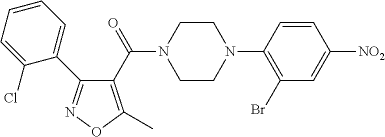

The title compound was synthesized by analogy to Example 6, substituting 2-bromo-1-chloro-4-nitrobenzene for 2-chloro-3-methoxy-5-nitropyridine. ¹H-NMR (CDCl₃, 500 MHz) δ 8.93 (1H, s), 8.14 (1H, s), 7.55 (1H, d, J=7.6), 7.51 (1H, d, J=7.9), 7.39-7.46 (2H, m), 3.76 (2H, s), 3.36 (4H, s), 2.96 (2H, s), 2.60 (3H, s), 2.31 (3H, s). HPLC/MS (Method K): (ES+) m/z (M+H)⁺=507; Rₜ=1.56 min.

### Examples 9-19

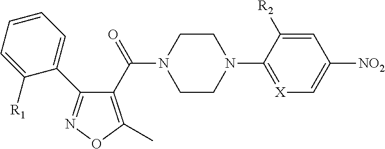

Examples 9-19 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | | | LC/MS | | |
|---|---|---|---|---|---|---|---|---|
| Example | R₁ | R₂ | X | MH+ | RT | Method | LHS Prep. | RHS Prep. |
| 9 | Br | Cl | CH | 506 | 3.03 | P | Commercial | Commercial |
| 10 | OCH₃ | Br | N | 504 | 2.28 | L | Acid-A | Amine-B |
| 11 | F | Cl | CH | 445 | 13.78 | O | Commercial | Commercial |
| 12 | CH₃ | Cl | CH | 441 | 5.68 | Q | Commercial | Commercial |
| 13 | CN | Cl | CH | 452 | 5.07 | Q | Acid-B | Commercial |
| 14 | OCH₂Ph | Cl | CH | 533 | 6.20 | Q | Acid-C | Commercial |
| 15 | CH₂CH₃ | Cl | CH | 455 | 6.01 | Q | Acid-D | Commercial |
| 16 | CF₃ | Cl | CH | 495 | 5.84 | Q | Acid-E | Commercial |
| 17 | OCHF₂ | Cl | CH | 493 | 5.52 | Q | Acid-F | Commercial |
| 18 | OCH₃ | Cl | CH | 457 | 2.92 | A | Acid-B | Commercial |
| 19 | OCH₃ | CF₃ | CH | 491 | 2.19 | F | Acid-A | Commercial |

### Examples 20-35

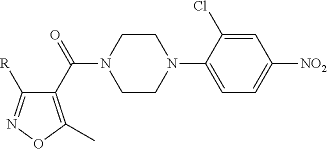

Examples 20-35 were synthesized by analogy to Example 1, substituting 1-(2-chloro-4-nitrophenyl)piperazine for 1-(2-methyl-4-nitrophenyl)piperazine and the appropriate LHS Preparation for 3-(2-chlorophenyl)-5-methylisoxazole-4-carboxylic acid.

| | | | | LC/MS | LHS |
|---|---|---|---|---|---|
| Example | R | MH+ | RT | Method | Prep. |
| 20 | 4-CH₃O—Ph | 457 | 6.28 | U | Commercial |
| 21 | 4-Cl—Ph | 461 | 7.27 | U | Commercial |
| 22 | 2,6-di-Cl—Ph | 495 | 6.46 | U | Commercial |
| 23 | 2-CH₃-3-F—Ph | 459 | 5.66 | Q | Acid-G |
| 24 | 2-Cl-6-F—Ph | 479 | 5.67 | Q | Acid-H |
| 25 | 2,3-di-CH₃O—Ph | 487 | 5.30 | Q | Acid-I |
| 26 | 3-CH₃—Ph | 441 | 3.14 | A | Commercial |
| 27 | 4-F—Ph | 445 | 3.00 | A | Commercial |
| 28 | 3-Cl—Ph | 461 | 2.56 | B | Commercial |
| 29 | 2-CH₃O-4-F—Ph | 475 | 1.80 | J | Acid-J |
| 30 | 2-Cl-4-F—Ph | 479 | 1.84 | J | Acid-K |
| 31 | 2-Cl-4-CH₃O—Ph | 491 | 2.42 | C | Commercial |
| 32 | 2,4-di-Cl—Ph | 497 | 2.39 | F | Commercial |
| 33 | 2,5-di-Cl—Ph | 495 | 1.32 | D | Acid-L |
| 34 | 2-CH₃O-5-Br—Ph | 537 | 1.57 | D | Acid-M |
| 35 | 4-N₃—Ph | 468 | 2.55 | C | Acid-BB |

### Examples 36-63

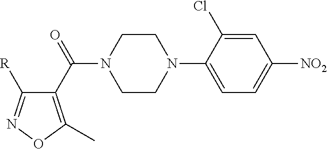

Examples 36-63 were synthesized by analogy to Example 1, substituting 1-(2-chloro-4-nitrophenyl)piperazine for 1-(2-methyl-4-nitrophenyl)piperazine and the appropriate LHS Preparation for 3-(2-chlorophenyl)-5-methylisoxazole-4-carboxylic acid.

| | | | | LC/MS | |
|---|---|---|---|---|---|
| Example | R | MH+ | RT | Method | LHS Prep |
| 36 | fur-2-yl | 417 | 2.46 | R | Commercial |
| 37 | 5-chloro-thiophen-2-yl | 467 | 3.63 | R | Commercial |
| 38 | 2-chloro-thiophen-3-yl | 467 | 2.89 | R | Commercial |
| 39 | quinolin-8-yl | 479 | 8.96 | O | Acid-N |
| 40 | pyrid-2-yl | 428 | 4.91 | Q | Acid-O |
| 41 | pyrimid-5-yl | 429 | 4.27 | Q | Acid-P |
| 42 | 3-methylpyrid-2-yl | 442 | 5.06 | Q | Acid-Q |
| 43 | 3,5-dimethylisoxazol-4-yl | 446 | 5.07 | Q | Acid-R |
| 44 | 3-methylthiophen-2-yl | 447 | 5.39 | Q | Acid-S |
| 45 | 2-methoxypyrid-3-yl | 458 | 4.88 | Q | Acid-T |
| 46 | 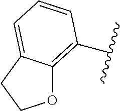 | 469 | 5.21 | Q | Acid-U |
| 47 | 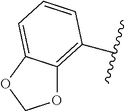 | 471 | 2.43 | S | Acid-V |
| 48 | 2-ethoxypyrid-3-yl | 472 | 5.38 | Q | Acid-W |
| 49 | naphth-1-yl | 477 | 4.88 | Q | Acid-X |
| 50 | quinolin-5-yl | 478 | 4.84 | Q | Acid-Y |
| 51 | isoquinolin-5-yl | 478 | 4.55 | Q | Acid-Z |
| 52 | quinolin-4-yl | 478 | 4.79 | Q | Acid-AA |
| 53 | 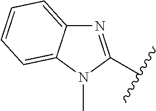 | 481 | 5.48 | Q | Acid-AB |
| 54 | 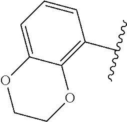 | 485 | 5.33 | Q | Acid-AC |
| 55 | 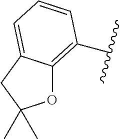 | 497 | 5.94 | Q | Acid-AD |
| 56 | 2-(pyrid-3-yl)phenyl | 504 | 5.09 | Q | Acid-AE |
| 57 | 3-(pyrid-3-yl)phenyl | 504 | 4.91 | Q | Acid-AF |
| 58 | pyrid-3-yl | 428 | 2.45 | A | Commercial |
| 59 | pyrid-4-yl | 428 | 2.31 | A | Commercial |
| 60 | 3-chloropyrid-4-yl | 462 | 1.90 | F | Acid-AG |
| 61 | 2-chloropyrid-3-yl | 462 | 2.74 | A | Acid-AH |
| 62 | quinoxalin-5-yl | 479 | 1.76 | I | Acid-AI |
| 63 | 2-methoxynaphth-1-yl | 507 | 2.48 | C | Acid-AJ |

### Examples 64-69

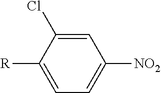

Examples 64-69 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | LC/MS | LHS | RHS |
|---|---|---|---|---|---|---|
| Example | R | MH+ | RT | Method | Prep. | Prep. |
| 64 | 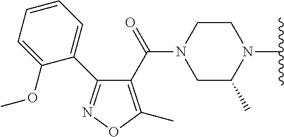 | 471 | 1.52 | K | Acid-A | Amine-C |
| 65 | 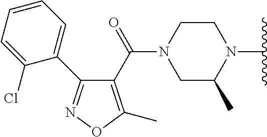 | 475 | 1.60 | K | Commercial | Amine-D |
| 66 | 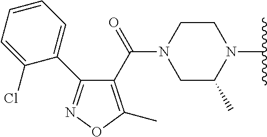 | 475 | 1.59 | K | Commercial | Amine-C |
| 67 | 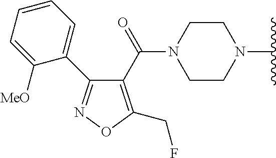 | 475 | 2.30 | C | Acid-AK | Commercial |
| 68 | 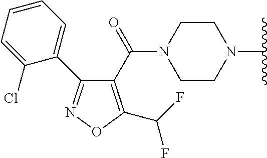 | 497 | 3.11 | A | Acid-AL | Commercial |
| 69 | 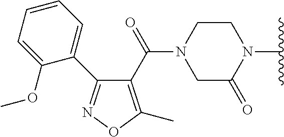 | 471 | 1.81 | C | Acid-A | Amine-G |

### Examples 70-90

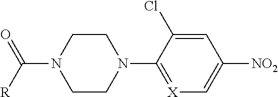

Examples 70-90 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | | LC/MS | | |
|---|---|---|---|---|---|---|---|
| Example | X | R | MH+ | RT | Method | LHS Prep. | RHS Prep. |
| 70 | CH | 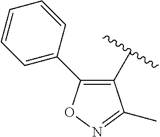 | 495 | 6.62 | V | Commercial | Commercial |
| 71 | CH | 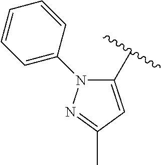 | 426 | 3.66 | V | Commercial | Commercial |
| 72 | CH | 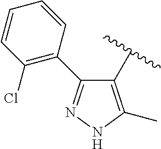 | 460 | 2.56 | T | Acid-AM | Commercial |
| 73 | CH | 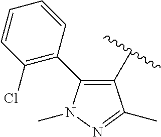 | 474 | 3.03 | R | Acid-AN | Commercial |
| 74 | CH | 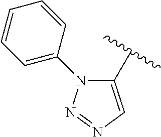 | 413 | 2.77 | R | Commercial | Commercial |
| 75 | CH | 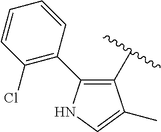 | 459 | 8.09 | O | Acid-AO | Commercial |
| 76 | CH | 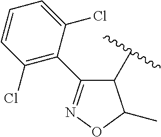 | 497 | 9.73 | O | Acid-AP | Commercial |
| 77 | CH | 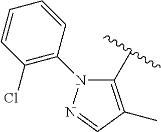 | 460 | 7.95 | O | Acid-AQ | Commercial |
| 78 | CH | 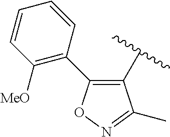 | 457 | 3.19 | R | Acid-AR | Commercial |
| 79 | CH | 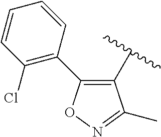 | 461 | 3.41 | R | Acid-AS | Commercial |
| 80 | CH | 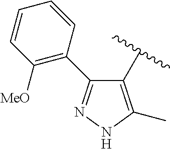 | 456 | 3.59 | R | Acid-AT | Commercial |
| 81 | CH | 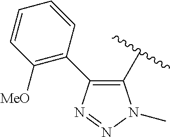 | 457 | 2.97 | G | Acid-AU | Commercial |
| 82 | CH | 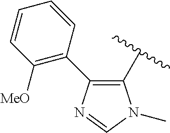 | 456 | 2.24 | G | Acid-AV | Commercial |
| 83 | N | 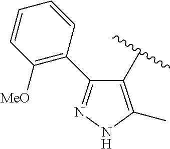 | 457 | 2.56 | G | Acid-AT | Amine-A |
| 84 | N | 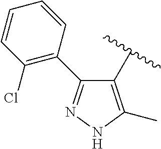 | 461 | 2.71 | G | Acid-AM | Amine-A |
| 85 | N | 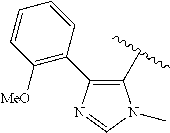 | 458 | 2.04 | G | Acid-AV | Amine-A |
| 86 | N | 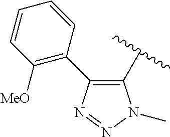 | 458 | 2.79 | G | Acid-AU | Amine-A |
| 87 | CH | 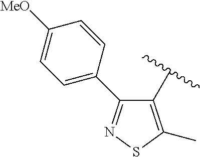 | 473 | 3.09 | A | Acid-AW | Commercial |
| 88 | CH | 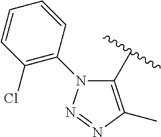 | 461 | 2.25 | C | Acid-AX | Commercial |
| 89 | CH | 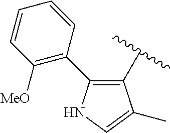 | 455 | 1.98 | I | Acid-AY | Commercial |
| 90 | CH | 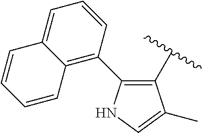 | 475 | 2.10 | I | Acid-AZ | Commercial |

### Example 91-97

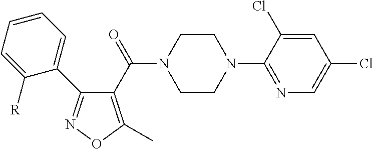

Examples 91-97 were synthesized by analogy to Example 1, substituting 1-(3,5-dichloro-piperidin-2-yl)piperazine for 1-(2-methyl-4-nitrophenyl)piperazine and the appropriate LHS Preparation for 3-(2-chlorophenyl)-5-methylisoxazole-4-carboxylic acid.

| | | | | LC/MS | |
|---|---|---|---|---|---|
| Example | R | MH+ | RT | Method | LHS Prep. |
| 91 | CH₃ | 431 | 3.61 | R | Commercial |
| 92 | F | 435 | 5.75 | R | Commercial |
| 93 | CF₂H | 483 | 5.82 | R | Acid-F |
| 94 | CN | 442 | 3.22 | R | Acid-B |
| 95 | Br | 497 | 2.57 | C | Commercial |
| 96 | OCH₃ | 447 | 2.20 | F | Acid-A |
| 97 | Cl | 451 | 3.12 | A | Commercial |

### Examples 98-118

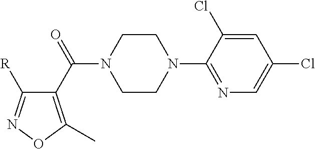

Examples 98-118 were synthesized by analogy to Example 1, substituting 1-(3,5-dichloro-piperidin-2-yl)piperazine for 1-(2-methyl-4-nitrophenyl)piperazine and the appropriate LHS Preparation for 3-(2-chlorophenyl)-5-methylisoxazole-4-carboxylic acid.

| | | | | LC/MS | |
|---|---|---|---|---|---|
| Example | Ar | MH+ | RT | Method | LHS Prep. |
| 98 | quinolin-8-yl | 468 | 8.95 | O | Acid-N |
| 99 | isoquinolin-5-yl | 468 | 8.05 | O | Commercial |
| 100 | pyrid-2-yl | 418 | 5.14 | Q | Acid-O |
| 101 | 3-methylpyrid-2-yl | 418 | 5.81 | R | Acid-Q |
| 102 | 3-methylthiophen-2-yl | 437 | 3.54 | R | Acid-S |
| 103 | 2-methoxypyrid-3-yl | 448 | 3.10 | R | Acid-T |
| 104 | 3-fluoro-2-methylphenyl | 449 | 3.68 | R | Acid-G |
| 105 | 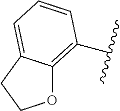 | 459 | 3.39 | R | Acid-U |
| 106 | 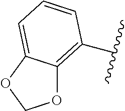 | 461 | 3.30 | R | Acid-V |
| 107 | 2-ethoxypyrid-3-yl | 462 | 3.38 | R | Acid-W |
| 108 | naphth-1-yl | 467 | 3.80 | R | Acid-X |
| 109 | quinolin-4-yl | 468 | 3.10 | R | Acid-AA |
| 110 | quinolin-5-yl | 468 | 2.96 | R | Acid-Y |
| 111 | 2-chloro-6-fluorophenyl | 469 | 6.00 | Q | Acid-H |
| 112 | 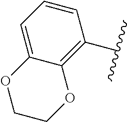 | 475 | 3.28 | R | Acid-AC |
| 113 | 2,3-dimethoxyphenyl | 477 | 3.34 | R | Acid-I |
| 114 | 2-methoxy-4- | 465 | 1.89 | J | Acid-J |
| | fluorophenyl | | | | |
| 115 | quinoxalin-5-yl | 469 | 1.83 | I | Acid-AI |
| 116 | 2-chloro-6- | 481 | 2.31 | F | Commercial |
| | methoxyphenyl | | | | |
| 117 | 2-methoxy-5- | 492 | 2.36 | C | Acid-BA |
| | nitrophenyl | | | | |
| 118 | 2-methoxynaphth-1-yl | 497 | 2.46 | I | Acid-AJ |

### Examples 119-125

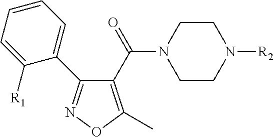

Examples 119-124 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | | LC/MS | | |
|---|---|---|---|---|---|---|---|
| Example | R₁ | R₂ | MH+ | RT | Method | LHS Prep. | RHS Prep. |
| 119 | Cl | 2-F-4-Cl—Ph | 434 | 1.69 | M | Commercial | Commercial |
| 120 | OCH₃ | 2-F-4-Cl—Ph | 430 | 1.49 | K | Acid-A | Commercial |
| 121 | Cl | 2-Cl-4-CN—Ph | 441 | 2.23 | T | Commercial | Commercial |
| 122 | Cl | 2,4-di-CH₃—Ph | 410 | 3.13 | A | Commercial | Commercial |
| 123 | Cl | 2-CH₃-4-Cl—Ph | 430 | 3.18 | A | Commercial | Commercial |
| 124 | Cl | 2,4-di-Cl—Ph | 451 | 3.25 | A | Commercial | Commercial |

### Examples 125-132

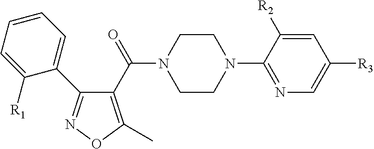

Examples 125-132 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | | | LC/MS | | |
|---|---|---|---|---|---|---|---|---|
| Example | R1 | R2 | R3 | MH+ | RT | Method | LHS Prep. | RHS Prep. |
| 125 | Cl | F | Cl | 435 | 1.51 | K | Commercial | Amine-I |
| 126 | Cl | Cl | CH₂OH | 447 | 1.05 | K | Commercial | Amine-J |
| 127 | Cl | Cl | Br | 495 | 1.61 | K | Commercial | Amine-K |
| 128 | Cl | Br | Br | 539 | 1.64 | K | Commercial | Amine-L |
| 129 | OCH₃ | Br | Cl | 493 | 1.54 | K | Acid-A | Amine-M |
| 130 | OCH₃ | Cl | Br | 493 | 1.51 | K | Acid-A | Amine-N |
| 131 | OCH₃ | Br | Br | 537 | 1.55 | K | Acid-A | Amine-L |
| 132 | Cl | CH₃ | Br | 477 | 2.99 | A | Commercial | Commercial |

### Examples 133-137

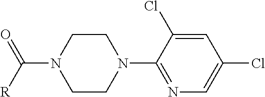

Examples 133-137 were synthesized by analogy to Example 1, substituting the appropriate RHS Preparation for 1-(2-methyl-4-nitrophenyl)piperazine and the appropriate LHS Preparation for 3-(2-chlorophenyl)-5-methylisoxazole-4-carboxylic acid.

| | | | | LC/MS | LHS |
|---|---|---|---|---|---|
| Example | R | MH+ | RT | Method | Prep |
| 133 | 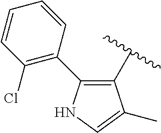 | 449 | 3.47 | R | Acid-AO |
| 134 | 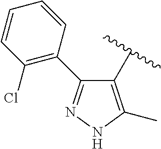 | 450 | 3.03 | R | Acid-AM |
| 135 | 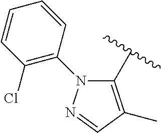 | 450 | 7.95 | O | Acid-AQ |
| 136 | 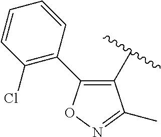 | 451 | 3.66 | R | Acid-AS |
| 137 | 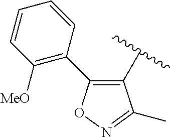 | 447 | 3.4 | R | Acid-AR |

### Example 138

### (3-(2-bromophenyl)-5-methylisoxazol-4-yl)(4-(2-chloro-4-nitrophenyl)piperazin-1-yl)methanone

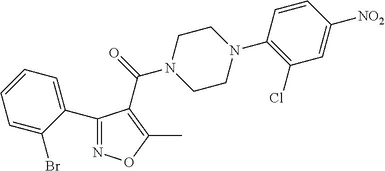

Reference: Xin, Z.; Zhao, H.; Serby, M. D. et al. Bioorg. Med. Chem. Lett. 2005, 15(4), 1201-1204.

To a solution of 1-(4-(2-chloro-4-nitrophenyl)piperazin-1-yl)butane-1,3-dione (200 mg, 0.614 mmol), in THF (3 mL) was added lithium bis(trimethylsilyl)amide (0.614 mL, 0.614 mmol) and stirred at room temperature for 1 h. To this pale yellow suspension was added (Z)-2-bromo-N-hydroxybenzimidoyl chloride (144 mg, 0.614 mmol), followed by Acetonitrile (3.00 mL) and the contents were stirred at rt overnight. Solid separated from the reaction mixture was filtered and dried to yield the title compound ¹H-NMR (CDCl₃, 500 MHz): δ 8.23 (1H, S), 8.05-8.12 (1H, m), 7.64-7.74 (1H, m), 7.40-7.53 (2H, m), 7.31-7.4 (1H, S), 6.75-6.91 (1H, m), 2.6 (3H, s). HPLC/MS (Method P): (ES+) m/z (M+H)⁺=506; Rₜ=3.03 min.

### Example 139

### (3-(2-(1H-pyrrol-2-yl)phenyl)-5-methylisoxazol-4-yl)(4-(2-chloro-4-nitrophenyl)piperazin-1-yl)methanone

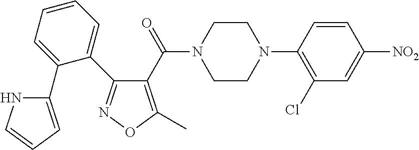

### Step 139A

### (4-(2-chloro-4-nitrophenyl)piperazin-1-yl)(3-(2-iodophenyl)-5-methylisoxazol-4-yl)methanone

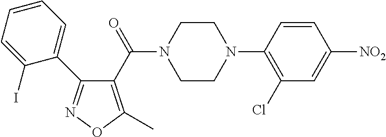

(4-(2-chloro-4-nitrophenyl)piperazin-1-yl)(3-(2-iodophenyl)-5-methylisoxazol-4-yl)methanone was prepared by analogy to Example 1, substituting 3-(2-iodophenyl)-5-methylisoxazole-4-carboxylic acid for 3-(2-chlorophenyl)-5-methylisoxazole-4-carboxylic acid and 1-(2-chloro-4-nitrophenyl)piperazine for 1-(2-methyl-4-nitrophenyl)piperazine. ¹H-NMR (CDCl₃, 500 MHz): δ 8.25 (1H, d, J=2.8 Hz), 8.10 (1H, dd, J1=9.0 Hz, J2=2.8 Hz), 8.00 (1H, d, J=8.9 Hz), 7.51-7.41 (2H, m), 7.22-7.16 (1H, m), 6.85 (1H, d, J=8.9 Hz), 3.81 (2H, s), 3.40 (2H, s), 3.04 (2H, s), 3.63 (3H, s), 2.55 (2H, s).

### Step 139B

The title compound was prepared by Suzuki coupling of (4-(2-chloro-4-nitrophenyl)piperazin-1-yl)(3-(2-iodophenyl)-5-methylisoxazol-4-yl)methanone and 1-(tert-butoxycarbonyl)-1H-pyrrol-2-ylboronic acid (Pd(PPh₃)₄, K₃PO₄ in 1,4-dioxane and water, 85° C.), followed by removal of Boc by treating the crude product with trifluoroacetic acid. ¹H-NMR (CD₃OD, 500 MHz): δ 10.57 (1H, s), 8.25 (1H, d, J=2.8 Hz), 8.15 (1H, dd, J1=9.0 Hz, J2=2.8 Hz), 7.58-7.49 (2H, m), 7.44 (1H, d, J=8.6 Hz), 7.39-7.32 (1H, m), 7.09 (1H, d, J=8.9 Hz), 6.81-6.75 (1H, m), 6.10-6.03 (1H, m), 5.92-5.86 (1H, m), 3.54 (2H, s), 3.13 (2H, s), 3.02 (2H, s), 2.54 (2H, s), 2.50 (3H, s).

### Example 140

### (4-(2-chloro-4-nitrophenyl)piperazin-1-yl)(2-(2-chlorophenyl)-1,4-dimethyl-1H-pyrrol-3-yl)methanone

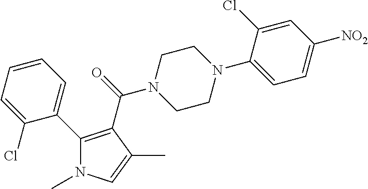

A mixture of (4-(2-chloro-4-nitrophenyl)piperazin-1-yl)(2-(2-chlorophenyl)-4-methyl-1H-pyrrol-3-yl)methanone (Example 75, 100 mg, 0.22 mmol), dimethyl carbonate (1 mL, 12 mmol) and DMF (0.1 mL) in presence of 1,4-Diazabicyclo[2.2.2]octane (2.4 mg, 0.022 mmol) was heated at 95° C. for 3 h. Solvent was removed using rotary evaporator and the residue was purified by Prep-HPLC to afford the title compound (30 mg, 0.22 mmol, 28% yield) as a pale yellow solid. ¹H NMR (CDCl₃, 400 MHz): δ 8.18 (1H, d, J=2.8 Hz), 7.92-8.06 (1H, m), 7.46 (1H, d, J=2.0 Hz), 7.32-7.41 (1H, m), 7.26-7.33 (1H, m), 7.23 (1H, s), 6.73-6.86 (1H, m), 6.44-6.57 (1H, m), 3.67 (2H, br. s.), 3.52 (2H, br. s.), 3.42-3.61 (2H, m), 3.37 (3H, s), 2.82 (2H, br. s.), 2.11 (3H, s). HPLC/MS (Method O): (ES+) m/z (M+H)⁺=473; Rₜ=11.83 min.

### Example 141

### (4-(2-bromo-6-nitropyridin-3-yl)piperazin-1-yl)(3-(2-methoxyphenyl)-5-methylisoxazol-4-yl)methanone

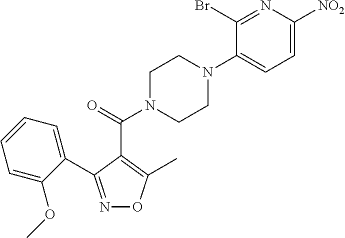

### Step 141A

### 5-Bromo-2-nitropyridin-3-amine

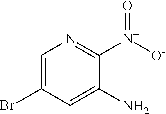

5-Bromo-2-nitropyridin-3-amine was prepared as described in reference: Journal of Medicinal Chemistry, 2007, 50, 18, 4453.

### Step 141B

### t-Butyl 4-(5-amino-6-nitropyridin-3-yl)piperazine-1-carboxylate

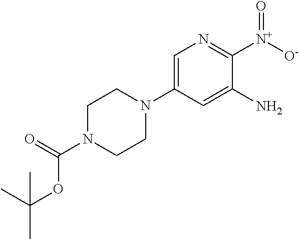

A mixture of 5-bromo-2-nitropyridin-3-amine (160 mg, 0.734 mmol) and t-butyl piperazine-1-carboxylate (1 g, 5.37 mmol) was stirred at 105° C. for 4 h. The reaction was cooled down to room temperature and diluted with water. The resulting precipitate was collected by filtration, washed with water to give the title compound (221 mg, 84%). 1H NMR (400 MHz, CHLOROFORM-d) δ ppm 7.70 (d, J=2.6 Hz, 1H), 6.31 (d, J=2.6 Hz, 1H), 5.99 (br. s., 2H), 3.70-3.58 (m, 4H), 3.43-3.27 (m, 4H), 1.50 (s, 9H).

### Step 141C

### t-Butyl 4-(5-amino-2-bromo-6-nitropyridin-3-yl)piperazine-1-carboxylate

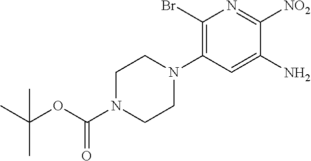

A solution of NBS (110 mg, 0.619 mmol) in dichloroethane (30 ml) was added dropwise to t-butyl 4-(5-amino-6-nitropyridin-3-yl)piperazine-1-carboxylate (200 mg, 0.619 mmol) in dichloroethane (35 ml) at 60° C. over 1 h. The resulted reaction mixture was stirred at room temperature for 15 mins. The solvent was evaporated under reduced pressure and the residue was purified by flash chromatography to give the title compound (88 mg, 35.4%). 1H NMR (500 MHz, CHLOROFORM-d) δ ppm 6.59 (s, 1H), 6.06 (br. s., 2H), 3.68-3.58 (m, 4H), 3.15-3.06 (m, 4H), 1.48 (s, 9H).

### Step 141D

### 6-Bromo-2-nitro-5-(piperazin-1-yl)pyridin-3-amine

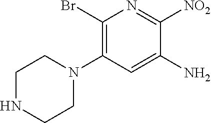

t-Butyl 4-(5-amino-2-bromo-6-nitropyridin-3-yl)piperazine-1-carboxylate (88 mg, 0.219 mmol) was treated with 50% TFA in dichloromethane (2 ml). The reaction mixture was stirred at room temperature for 0.5 h. The solvent was evaporated and the residue was dried under vacuum pump to give the title compound (85 mg, 93%). LCMS—Phenomenex Luna C18 3.0×50 mm S10, 0 to 100% B over 2.0 minute gradient, 1 minute hold time, A=5% acetonitrile 95% water 10 mM NH₄OAc, B=95% acetonitrile 5% water 10 mM NH₄OAc. Flow rate: 4 ml/min. Retention time: 0.793 min, m/e 302.10 (M+1)⁺.

### Step 141E

### (4-(5-Amino-2-bromo-6-nitropyridin-3-yl)piperazin-1-yl)(3-(2-methoxyphenyl)-5-methylisoxazol-4-yl)methanone

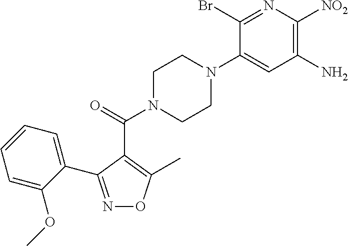

To a solution of 6-bromo-2-nitro-5-(piperazin-1-yl)pyridin-3-amine, TFA salt (85 mg, 0.204 mmol) in DMF (3 ml) was added DIEA (0.143 ml, 0.817 mmol), HATU (85 mg, 0.225 mmol) and 3-(2-methoxyphenyl)-5-methylisoxazole-4-carboxylic acid (47.6 mg, 0.204 mmol). The reaction was stirred at room temperature for 1 h. The reaction mixture was purified by preparative HPLC to afford 73 mg (68.4%) of the title compound. 1H NMR (500 MHz, CHLOROFORM-d) δ ppm 7.58 (dd, J=7.63, 1.83 Hz, 1H), 7.46 (td, J=7.93, 1.83 Hz, 1H), 7.10-1.07 (m, 1H), 6.98 (d, J=8.55 Hz, 1H), 6.38 (s, 1H), 6.02 (br. s., 2H), 3.92-3.71 (m, 5H), 3.24-3.22 (m, 2H), 3.03-3.02 (m, 2H), 2.57 (s, 3H), 2.46-2.43 (m, 2H).

### Step 141F

The suspension of (4-(5-amino-2-bromo-6-nitropyridin-3-yl)piperazin-1-yl)(3-(2-methoxyphenyl)-5-methylisoxazol-4-yl)methanone (25 mg, 0.048 mmol) in Ethanol (500 μL) was cooled down to −10° C. 48% HBF4 (150 ul) was added in one portion. The temperature was further lowered to −25° C. Isoamyl nitrite (50 μL, 0.371 mmol) was added dropwise. The reaction was allowed to warm up to −5° C. The reaction mixture was stirred at −5° C. for 30 mins. Then it was cooled down to −25° C. 50% Aqueous H3PO2 (500 ul) was added dropwise. The reaction was allowed to warm up to room temperature slowly and stirred at room temperature for 16 h. DMF(1 ml) was added to the reaction mixture. After filtration, the filtrate was purified by preparative HPLC to afford the title compound (8 mg, 31.3%). ¹H NMR (500 MHz, CHLOROFORM-d) δ ppm 8.17 (d, J=8.24 Hz, 1H), 7.59 (dd, J=7.48, 1.98 Hz, 1H), 7.43-7.51 (m, 1H), 7.17 (d, J=8.55 Hz, 1H), 7.05-7.13 (m, 1H), 6.99 (d, J=8.24 Hz, 1H), 3.86 (br. s., 2H), 3.79 (s, 3H), 3.28 (br. s., 2H), 3.09 (br. s., 2H), 2.57 (s, 3H), 2.44 (br. s., 2H). HPLC/MS (Method D): (ES+) m/z (M+H)⁺=502; Rₜ=1.28 min.

### Example 142

### (4-(3,5-dichloropyridin-2-yl)piperazin-1-yl)(3-(2-hydroxyphenyl)-5-methylisoxazol-4-yl)methanone

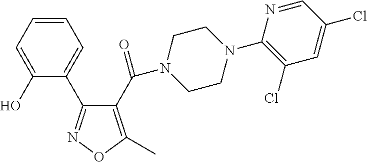

A 1.0 M solution of boron tribromide (5.70 mL, 5.70 mmol) was cooled to −78° C. A solution of (4-(3,5-dichloropyridin-2-yl)piperazin-1-yl)(3-(2-methoxyphenyl)-5-methylisoxazol-4-yl)methanone (Example 96, 0.85 g, 1.900 mmol) in DCM (10 mL) was added dropwise. The reaction mixture was stirred at −78° C. for 3 hr. and at room temperature overnight. Water was added slowly, followed by the addition of saturated NaHCO₃ solution. The product was extracted with DCM (3×50 mL). The crude product was purified via column chromatography (20% EtOAc/DCM R_{f} 0.45) to give 0.75 g (92%, theoretical yield 0.823 g) of the title compound. ¹H-NMR (CDCl₃, 300 MHz): δ 9.31 (1H, s), 8.12 (1H, d, J=2.2 Hz), 7.62 (1H, d, J=2.2 Hz), 7.50-7.40 (1H, m), 7.40-7.30 (1H, m), 7.14-7.06 (1H, m), 6.99-6.87 (1H, m), 3.98 (2H, t, J=5.1 Hz), 3.40 (2H, t, J=5.1 Hz), 3.35 (2H, s), 3.05 (2H, s), 2.54 (3H, s). HPLC/MS (Method F): (ES+) m/z (M+H)⁺=433; Rₜ=2.05 min.

### Example 143

### (4-(3,5-dichloropyridin-2-yl)piperazin-1-yl)(3-(2-isopropoxyphenyl)-5-methylisoxazol-4-yl)methanone

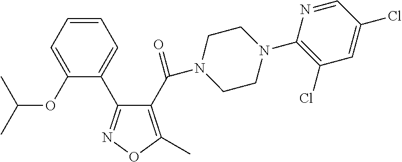

To a solution of (4-(3,5-dichloropyridin-2-yl)piperazin-1-yl)(3-(2-hydroxyphenyl)-5-methylisoxazol-4-yl)methanone (Example 142, 20 mg, 0.046 mmol), propan-2-ol (4.16 mg, 0.069 mmol) and tri-n-butylphosphine (0.017 mL, 0.069 mmol) in THF (2 mL) at 0° C. was added diisopropyl azodicarboxylate (0.013 mL, 0.069 mmol). The reaction mixture was stirred at 0° C. for 3 minutes and then at room temperature overnight. Solvent was evaporated in vacuo. The product was purified by prep HPLC (0.1% TFA buffer, MeOH/H2O) to give 13.0 mg (59% yield). ¹H-NMR (CD₃OD, 300 MHz): δ 8.11 (1H, d, J=2.2 Hz), 7.77 (1H, d, J=2.2 Hz), 7.51-7.41 (2H, m), 7.15-6.99 (2H, m), 4.64 (1H, m), 3.70 (2H, s), 3.26-3.08 (4H), 2.57 (2H, s), 2.54 (3H, s), 1.27 (6H, d, J=5.9 Hz).

### Examples 144-146

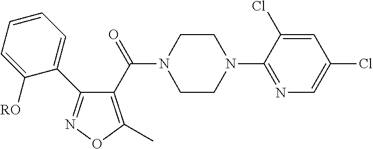

Examples 144-146 were synthesized by analogy to Example 143, substituting the appropriate alcohol (“R—OH”) for propan-2-ol.

| | | | | LC/MS |
|---|---|---|---|---|
| Example | R | MH+ | RT | Method |
| 144 | Et | 461 | 2.35 | F |
| 145 | nPr | 475 | 2.74 | C |
| 146 | CH₂CH₂OCH₃ | 491 | 2.43 | C |

### Example 147

### (4-(2-chloro-4-nitrophenyl)piperazin-1-yl)(3-(2-hydroxyphenyl)-5-methylisoxazol-4-yl)methanone

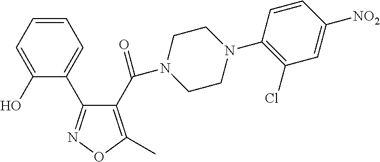

The title compound was prepared by analogy to Example 142, substituting Example 18 for Example 96. ¹H-NMR (CDCl₃, 500 MHz): δ 9.20 (1H, s), 8.26 (1H, d, J=2.7 Hz), 8.10 (1H, dd, J1=9.0 Hz, J2=2.7 Hz), 7.47-7.42 (1H, m), 7.40-7.34 (1H, m), 7.12 (1H, d, J=8.6 Hz), 6.99-6.90 (2H, m), 4.03 (2H, s), 3.37 (2H, s), 3.23 (2H, s), 2.69 (2H, s), 2.56 (3H, s). HPLC/MS (Method C): (ES+) m/z (M+H)⁺=443; Rₜ=2.16 min.

### Examples 148-153

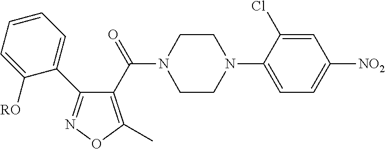

Examples 148-153 were synthesized by analogy to Example 143, substituting Example 147 for Example 142 and the appropriate alcohol (“R—OH”) for propan-2-ol.

| | | | | LC/MS |
|---|---|---|---|---|
| Example | R | MH+ | RT | Method |
| 148 | nPr | 485 | 3.18 | E |
| 149 | iPr | 485 | 2.33 | F |
| 150 | nBu | 499 | 2.48 | F |
| 151 | CH₂CH₂N(CH₃)₂ | 514 | 1.59 | C |
| 152 | CH₂CH₃ | 471 | 2.43 | C |
| 153 | CH₂CH₂OCH₃ | 501 | 2.32 | C |

### Example 154

### Methyl 2-(4-(4-(2-chloro-4-nitrophenyl)piperazine-1-carbonyl)-5-methylisoxazol-3-yl)benzoate

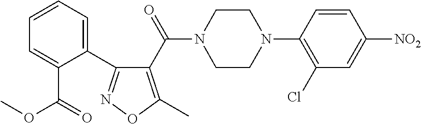

The title compound was synthesized by analogy to Example 1, substituting 1-(2-chloro-4-nitrophenyl)piperazine for 1-(2-methyl-4-nitrophenyl)piperazine and Acid-BC for 3-(2-chlorophenyl)-5-methylisoxazole-4-carboxylic acid. ¹H-NMR (CD₃OD, 500 MHz): δ 8.23 (1H, d, J=2.6 Hz), 8.12 (1H, dd, J1=8.9 Hz, J2=2.6 Hz), 8.00-7.94 (1H, m), 7.75-7.62 (2H, m), 7.58-7.52 (1H, m), 7.03 (1H, d, J=9.2 Hz), 3.80 (3H, s), 2.56 (3H, s). HPLC/MS (Method I): (ES+) m/z (M+H)⁺=485; Rₜ=2.06 min.

### Example 155

### 2-(4-(4-(2-chloro-4-nitrophenyl)piperazine-1-carbonyl)-5-methylisoxazol-3-yl)benzamide

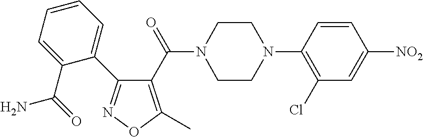

### Step 155A

### 2-(4-(4-(2-chloro-4-nitrophenyl)piperazine-1-carbonyl)-5-methylisoxazol-3-yl)benzoic acid

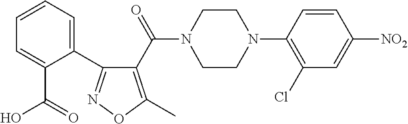

A solution of methyl 2-(4-(4-(2-chloro-4-nitrophenyl)piperazine-1-carbonyl)-5-methylisoxazol-3-yl)benzoate (Example 154, 496 mg, 1.022 mmol) and sodium hydroxide (245 mg, 6.13 mmol) in methanol (15 mL) and Water (1.5 mL) was stirred at room temperature overnight. The reaction mixture was concentrated by removing methanol by rotary evaporation. The concentrated reaction mixture was transferred to a 250-mL separatory funnel with 80 mL of water and 60 mL of ethyl acetate. Two layers were separated and the organic layer was discarded. The aqueous layer was acidified by adding concentrated HCl (0.8 mL). The product was extracted with ethyl acetate (2×60 mL) and methylene chloride (2×50 mL). The extract was dried over anhydrous sodium sulfate and solvent was evaporated in vacuo to give 0.400 g (83%, theoretical yield 0.481 g). ¹H-NMR (DMSO-d6, 500 MHz): δ 13.05 (1H, s), 8.24 (1H, d, J=2.6 Hz), 8.17 (1H, dd, J1=9.2 Hz, J2=2.6 Hz), 7.95-7.87 (1H, m), 7.73-7.58 (2H, m), 7.49-7.40 (1H, m), 7.18 (1H, d, J=9.2 Hz), 2.52 (3H, s).

### Step 155B

2-(4-(4-(2-Chloro-4-nitrophenyl)piperazine-1-carbonyl)-5-methylisoxazol-3-yl)benzoyl chloride (44 mg, 0.090 mmol) was prepared from 2-(4-(4-(2-chloro-4-nitrophenyl)piperazine-1-carbonyl)-5-methylisoxazol-3-yl)benzoic acid by treatment with oxalyl chloride (17 mg, 0.135 mmol) in DCM (2.0 mL) at room temperature in the presence of 1 drop of DMF. Solvent was evaporated in vacuo, followed by the addition of DCM (2 mL) and ammonia (0.054 mL, 0.108 mmol) (2.0 M solution in methanol). The resulted reaction mixture was stirred at room temperature overnight. The product was purified by preparative HPLC (0.1% TFA MeOH/H2O) to give 4.5 mg of the title compound. ¹H-NMR (DMSO, 400 MHz): δ 8.24 (1H, d, J=2.6 Hz), 8.16 (1H, dd, J1=8.9 Hz, J2=2.6 Hz), 7.98 (1H, s), 7.69-7.61 (1H, m), 7.61-7.51 (2H, m), 7.45-7.34 (2H, m), 7.17 (1H, d, J=9.0 Hz), 2.51 (3H, s). HPLC/MS (Method F): (ES+) m/z (M+H)⁺=470; Rₜ=1.54 min.

### Examples 156-158

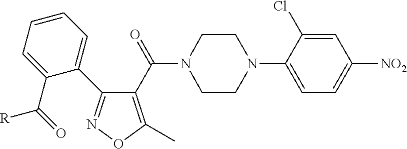

Examples 156-158 were synthesized by analogy to Example 155, substituting the appropriate amine (“R—NH₂”) for ammonia.

| | | | | LC/MS |
|---|---|---|---|---|
| Example | R | MH+ | RT | Method |
| 156 | NHCH₃ | 484 | 2.27 | H |
| 157 | NHPh | 546 | 2.44 | C |
| 158 | NHnPr | 512 | 2.19 | C |

### Example 159

### (3-(5-amino-2-methoxyphenyl)-5-methylisoxazol-4-yl)(4-(3,5-dichloropyridin-2-yl)piperazin-1-yl)methanone

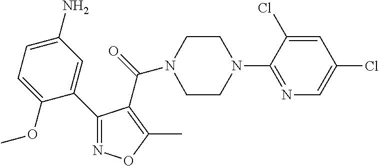

### Step 159A

### (4-(3,5-dichloropyridin-2-yl)piperazin-1-yl)(3-(2-methoxy-5-nitrophenyl)-5-methylisoxazol-4-yl)methanone

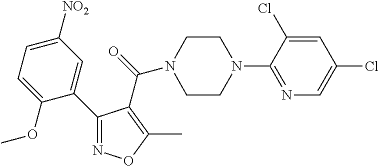

(4-(3,5-dichloropyridin-2-yl)piperazin-1-yl)(3-(2-methoxy-5-nitrophenyl)-5-methylisoxazol-4-yl)methanone was synthesized by analogy to Example 1, substituting 1-(3,5-dichloro-piperidin-2-yl)piperazine for 1-(2-methyl-4-nitrophenyl)piperazine and Acid-BA for 3-(2-chlorophenyl)-5-methylisoxazole-4-carboxylic acid.

### Step 159B

The title compound was prepared by analogy to Step BD2 of Acid-BD.

¹H-NMR (CD₃OD, 400 MHz): δ 8.15 (1H, d, J=2.3 Hz), 7.82 (1H, d, J=2.3 Hz), 7.59 (1H, d, J=2.8 Hz), 7.54 (1H, dd, J1=8.8 Hz, J2=2.8 Hz), 7.28 (1H, d, J=9.0 Hz), 3.86 (3H, s), 3.78 (2H, s), 3.40 (2H, s), 2.97 (2H, s), 2.56 (3H, s). HPLC/MS (Method J): (ES+) m/z (M+H)⁺=462; Rₜ=1.66 min.

### Example 160

### N-(3-(4-(4-(3,5-dichloropyridin-2-yl)piperazine-1-carbonyl)-5-methylisoxazol-3-yl)-4-methoxyphenyl)acetamide

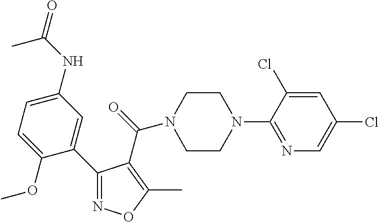

To a solution of (3-(5-amino-2-methoxyphenyl)-5-methylisoxazol-4-yl)(4-(3,5-dichloropyridin-2-yl)piperazin-1-yl)methanone (Example 159, 28 mg, 0.061 mmol) and DIEA (0.026 mL, 0.151 mmol) in DCM (1.4 mL) was added acetyl chloride (5 μL, 0.091 mmol). The resulting reaction mixture was stirred at room temperature for one hour. The product was purified by preparative HPLC (0.1% TFA, MeOH/H₂O) to give 16.6 mg (54% yield).

¹H-NMR (400 MHz, MeOD) δ 8.12 (1H, d, J=2.3 Hz), 7.76 (2H, dd, J=11.8, 2.5 Hz), 7.66 (1H, dd, J=8.9, 2.6 Hz), 7.07 (1H, d, J=9.0 Hz), 3.78 (3H, s), 3.75 (2H, br. s.), 3.28 (4H, br. s.), 2.81 (2H, br. s.), 2.53 (3H, s), 2.11 (3H, s). HPLC/MS (Method J): (ES+) m/z (M+H)⁺=504; Rₜ=1.67 min.

### Example 161

### N-(3-(4-(4-(3,5-dichloropyridin-2-yl)piperazine-1-carbonyl)-5-methylisoxazol-3-yl)-4-methoxyphenyl)methanesulfonamide

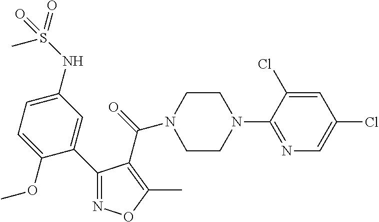

To a solution of (3-(5-amino-2-methoxyphenyl)-5-methylisoxazol-4-yl)(4-(3,5-dichloropyridin-2-yl)piperazin-1-yl)methanone (Example 159, 34.5 mg, 0.075 mmol) and methanesulfonyl chloride (7.0 μL, 0.090 mmol) in DCM (1.4 mL) was added 4-methylmorpholine (11.3 mg, 0.112 mmol). The resulted reaction mixture was stirred at room temperature for one hour. The product was purified by preparative HPLC (0.1% TFA, MeOH/H₂O) to give 8.8 mg (21% yield). ¹H-NMR (400 MHz, MeOD) δ 8.12 (1H, d, J=2.3 Hz), 7.80 (1H, d, J=2.3 Hz), 7.39-7.49 (2H, m), 7.12 (1H, d, J=8.8 Hz), 3.79 (3H, s), 3.75 (2H, br. s.), 3.25 (2H, br. s.), 2.96 (3H, s), 2.73 (2H, br. s.), 2.54 (3H, s). HPLC/MS (Method J): (ES+) m/z (M+H)⁺=540; Rₜ=1.65 min.

### Example 162

### (3-(5-amino-2-methoxyphenyl)-5-methylisoxazol-4-yl)(4-(2-chloro-4-nitrophenyl)piperazin-1-yl)methanone

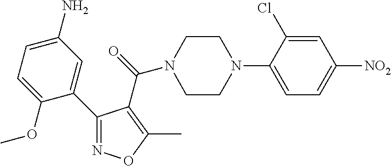

### Step 162A

### tert-butyl 3-(4-(4-(2-chloro-4-nitrophenyl)piperazine-1-carbonyl)-5-methylisoxazol-3-yl)-4-methoxyphenylcarbamate

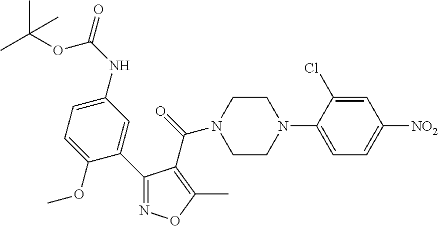

Tert-butyl 3-(4-(4-(2-chloro-4-nitrophenyl)piperazine-1-carbonyl)-5-methylisoxazol-3-yl)-4-methoxyphenylcarbamate was prepared by analogy to Example 1, substituting 1-(2-chloro-4-nitrophenyl)piperazine for 1-(2-methyl-4-nitrophenyl)piperazine and Acid-BD for 3-(2-chlorophenyl)-5-methylisoxazole-4-carboxylic acid.

### Step 162B

Tert-butyl 3-(4-(4-(2-chloro-4-nitrophenyl)piperazine-1-carbonyl)-5-methylisoxazol-3-yl)-4-methoxyphenylcarbamate was treated with TFA in DCM for 1 hour and concentrated in vacuo. The crude residue was purified by prep HPLC to provide the title compound. ¹H-NMR (CD₃OD, 400 MHz): δ 8.26 (1H, d, J=2.8 Hz), 8.15 (1H, dd, J1=9.0 Hz, J2=2.8 Hz), 7.60 (1H, d, J=2.8 Hz), 7.54 (1H, dd, J1=8.8 Hz, J2=2.8 Hz), 7.30 (1H, d, J=9.0 Hz), 7.19 (1H, d, J=9.0 Hz), 3.88 (3H, s), 3.83 (2H, s), 3.46 (2H, s), 3.21 (2H, s), 2.88 (2H, s), 2.57 (3H, s). HPLC/MS (Method J): (ES+) m/z (M+H)⁺=472; Rₜ=1.58 min.

### Examples 163-165

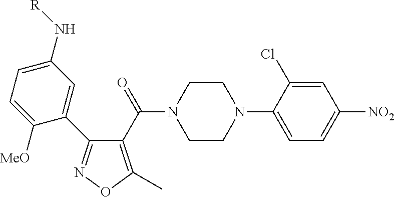

Examples 163-165 were prepared by analogy to Example 160, substituting Example 162 for Example 159 and the appropriate acylating agent (“R—Cl”) for acetyl chloride.

| | | | | LC/MS |
|---|---|---|---|---|
| Example | R | MH+ | RT | Method |
| 163 | SO₂Me | 550 | 1.63 | J |
| 164 | (C═O)CH₂N(CH₃)₂ | 557 | 1.66 | J |
| 165 | (C═O)Me | 514 | 2.14 | F |

### Example 166

### (4-(4-azido-2-chlorophenyl)piperazin-1-yl)(3-(2-chlorophenyl)-5-methylisoxazol-4-yl)methanone

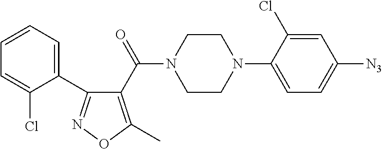

### Step 166A

### (4-(2-chloro-4-nitrophenyl)piperazin-1-yl)(3-(2-chlorophenyl)-5-methylisoxazol-4-yl)methanone

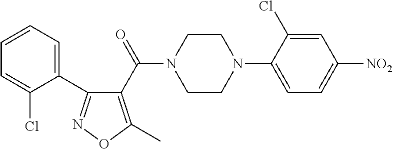

To 3-(2-chlorophenyl)-5-methylisoxazole-4-carboxylic acid (1.426 g, 6.00 mmol) in CH2Cl2 (40 mL) was added oxalyl chloride (0.914 g, 7.20 mmol) and 2 drops of DMF, then the mixture was stirred for 2 hours. After bubbling stopped, the solvent was removed by rotovap and dried in vacuum for 10 min, then the residue was dissolved in DCM (40 mL) and triethylamine (1.821 g, 18.00 mmol), 1-(2-chloro-4-nitrophenyl)piperazine (1.450 g, 6.00 mmol) were added, stirred overnight. LCMS showed the desired product is major. After solvent was removed by rotovap, a yellow solid was obtained, (4-(2-chloro-4-nitrophenyl)piperazin-1-yl)(3-(2-chlorophenyl)-5-methylisoxazol-4-yl)methanone (crude, 2.77 g, 6.00 mmol).

### Step 166B

### (4-(4-amino-2-chlorophenyl)piperazin-1-yl)(3-(2-chlorophenyl)-5-methylisoxazol-4-yl)methanone

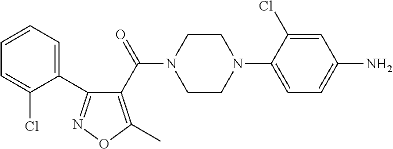

To a solution of (4-(2-chloro-4-nitrophenyl)piperazin-1-yl)(3-(2-chlorophenyl)-5-methylisoxazol-4-yl)methanone (2.77 g, 6.00 mmol) in DMF (60 ml) was added tin(II) chloride dihydrate (6.77 g, 30.0 mmol). The mixture was stirred overnight, then was treated with concentrated hydrochloride acid (20 ml). The solution was adjusted to pH at 5-6 with 50% NaOH. Then the mixture was extracted with EtOAc four times (4×50 ml) and combined extractions was washed with water (6×50 ml). After solvent was removed, the residue was dried in vacuum to give (4-(4-amino-2-chlorophenyl)piperazin-1-yl)(3-(2-chlorophenyl)-5-methylisoxazol-4-yl)methanone (2 g, 4.64 mmol, 77% yield).

¹H-NMR (CD₃OD, 500 MHz) δ 7.46-7.59 (4H, m), 7.35 (1H, s), 7.22 (1 h, d, J=8.6 Hz), 7.04 (1H, d, J=8.6 Hz), 3.76 (2H, s), 3.42 (2H, s), 2.95 (2H, s), 2.58 (3H, s), 2.51 (2H, s).

### Step 166C

A mixture of (4-(4-amino-2-chlorophenyl)piperazin-1-yl)(3-(2-chlorophenyl)-5-methylisoxazol-4-yl)methanone (43 mg, 0.100 mmol), tert-butyl nitrite (20.56 mg, 0.199 mmol), and azidotrimethylsilane (17.23 mg, 0.150 mmol) in acetonitrile (1 ml) was stirred at 0° C. for 2 h, then warmed up to RT and stand overnight. HPLC purification afforded the title compound (31.2 mg, 0.054 mmol, 54.2% yield).

¹H-NMR (CDCl₃, 500 MHz) δ 7.50-7.55 (2H, m), 7.37-7.46 (2H, m), 7.04 (1H, s), 6.88 (1H, d, J=8.5 Hz), 6.81 (1H, d, J=8.5 Hz), 3.78 (2H, s), 3.37 (2H, s), 2.89 (2H, s), 2.59 (3H, s), 243 (2H, s). HPLC/MS (Method K): (ES+) m/z (M+H)⁺=457; Rₜ=1.65 min.

### Example 167

### 6-(4-(3-(2-chlorophenyl)-5-methylisoxazole-4-carbonyl)piperazin-1-yl)-5-methylnicotinonitrile

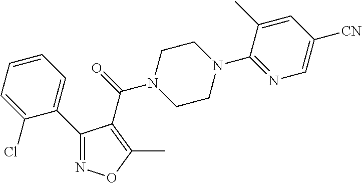

The title compound was prepared from Example 132 (4-(5-bromo-3-methylpyridin-2-yl)piperazin-1-yl)(3-(2-chlorophenyl)-5-methylisoxazol-4-yl)methanone as described in Tschaen, D. M.; Desmond, R.; King, A. O.; Fortin, M. C.; Pipik, B.; King, S.; Verhoeven, T. R. Synth. Commun., 1994, 24, 887-890.

¹H NMR (500 MHz, DMSO-d₆) δ 8.50 (1H, d, J=2.4 Hz), 7.90 (1H, d, J=1.5 Hz), 7.45-7.66 (4H, m), 3.60 (2H, br. s.), 3.44 (2H, br. s.), 3.23 (2H, br. s.), 3.02 (2H, br. s.), 2.55 (3H, s), 2.24 (3H, s). HPLC/MS (Method B): (ES+) m/z (M+H)⁺=422; Rₜ=2.12 min.

### Materials and Methods

### Cells and Virus

Madin Darby canine kidney (MDCK) cells and influenza A/WSN/33 were obtained from ATCC. Influenza A/Solomon Islands/3/06 and influenza A/Brisbane/10/2007 were obtained from the CDC.

### Compounds

Test compounds, at 100× the final test concentration, were serially diluted in DMSO in 3-fold steps. One ul of diluted compound was added to each well of a 96-well plate.

### Antivial Assays

For antiviral assays, MDCK cells were re-suspended in assay media (MEM with pen/strep plus 0.125% BA (bovine albumin) and 1 ug/ml TPCK-treated trypsin) at 4.5×10⁵ cells per ml. Virus was added for final multiplicity of infection (MOI) of 0.001 plaque forming units per cell and 100 ul was added to each well of a 96-well plate (1 ul of compound/well). For cytotoxicity assays, only cells were added to the assay plates. 48 hrs post infection, viral replication in the presence of inhibitor was determined by measuring viral neuraminidase (NA) activity via activation of the quenched substrate 2′-(4-Methylunbelliferyl)-α-D-N-acetylneuraminic acid (MUNANA). A 5× substrate solution was added to yield a final concentration of 100 uM MUNANA, 50 mM MES, 2 mM CaCl₂ and 0.25% NP-40. After a 30 minute incubation at 37° C. the plates were read on a fluorescence plate reader set at 360 nm excitation and 460 nm emission. Cytotoxicity was ascertained via crystal violet staining of treated cells. Cells were washed once with PBS, stained for 20 min with 0.5% crystal violet in 20% methanol, washed with water and air dried. 50 ul of methanol was added to each well to solubilize the crystal violet and 50 ul PBS was added before the absorbance was read at 540 nM.

### REFERENCES

Chen J, Deng Y M. 2009. Influenza virus antigenic variation, host antibody production and new approach to control epidemic. Virol J. March 13; 6:30.
Deyde V M, Sheu T G, Trujillo A A, Okomo-Adhiambo M, Garten R, Klimov A I, Gubareva L V. 2010. Detection of molecular markers of drug resistance in 2009 pandemic influenza A (H1N1) viruses by pyrosequencing. Antimicrob Agents Chemother. March; 54(3):1102-10.
Moscona A. 2009. Global transmission of oseltamivir-resistant influenza. N Engl J Med. March 5; 360(10):953-6.
Soepandi P Z, Burhan E, Mangunnegoro H, Nawas A, Aditama T Y, Partakusuma L, Isbaniah F, Malik S, Benamore R, Baird J K, Taylor W R. 2010. Clinical course of H5N1 avian influenza in patients at the Persahabatan Hospital, Jakarta, Indonesia, 2005-2008. Chest 09-2644.
Zimmer S M, Burke D S. 2009. Historical perspective—Emergence of influenza A (H1N1) viruses. N Engl J Med. July 16; 361(3):279-85.

Activity Table 1

| | | A/H1N1/ | | | | |
|---|---|---|---|---|---|---|
| | A/H1N1/ | Solomon | A/H3N2 | A/H5N1/ | A/H5N1/ | A/H5N1/ |
| Example | WSN | Islands | Brisbane | Duck_MN | Duck_PA | Gull_PA |
| 133 | ++ | + | + | − | − | + |
| 80 | + | + | − | + | + | ++ |
| 82 | ++ | − | − | + | + | ++ |
| 18 | +++ | ++ | + | ++ | ++ | +++ |
| 81 | +++ | + | − | ++ | ++ | +++ |
| 86 | ++ | + | − | + | + | ++ |
| 75 | +++ | + | − | ++ | ++ | +++ |
| 72 | ++ | − | − | + | + | ++ |
| 77 | ++ | − | − | + | + | ++ |
| 88 | ++ | + | − | + | + | ++ |
| 61 | +++ | + | − | ++ | ++ | ++ |
| 132 | ++ | + | − | − | − | − |
| 31 | +++ | ++ | − | ++ | ++ | +++ |
| 129 | ++ | + | + | + | + | ++ |
| 63 | +++ | ++ | − | ++ | ++ | +++ |
| 165 | ++ | ++ | − | + | + | ++ |

Table Key: “−” = EC₅₀ > 10 uM; “+” = EC₅₀ ≦ 10 uM; “++” = EC₅₀ < 1 uM; “+++” = EC₅₀ < 0.1 uM

ACTIVITY TABLE 2

| | Human Liver Microsomal | Mouse Liver Microsomal |
|---|---|---|
| | Stability (% Remaining | Stability (% Remaining |
| Example | after 10 minutes) | after 10 minutes) |
| 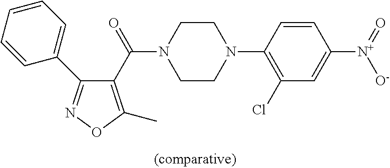 | 40 | 20 |
| 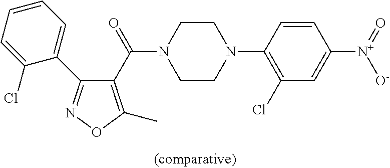 | 6 | 0 |
| 28 | 94 | 85 |
| 155 | 80 | 61 |
| 72 | 78 | 61 |
| 163 | 75 | 49 |
| 81 | 73 | 45 |
| 80 | 72 | 65 |
| 4 | 72 | 86 |
| 161 | 70 | 8 |
| 29 | 68 | 28 |
| 27 | 66 | 86 |
| 159 | 62 | 36 |
| 45 | 56 | 33 |
| 83 | 52 | 16 |
| 61 | 50 | 18 |
| 142 | 80 | 34 |
| 88 | 47 | 23 |
| 82 | 45 | 43 |
| 67 | 40 | 29 |

The foregoing description is merely illustrative and should not be understood to limit the scope or underlying principles of the invention in any way. Indeed, various modifications of the invention, in addition to those shown and described herein, will become apparent to those skilled in the art from the following examples and the foregoing description. Such modifications are also intended to fall within the scope of the appended claims.

What is claimed is:

1. A compound of Formula I, including pharmaceutically acceptable salts thereof: 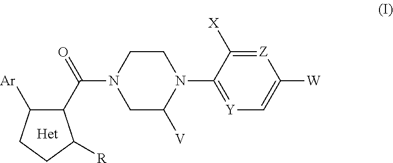
wherein Het is a 5 or 6-membered heterocycle with —N, —O, or —S adjacent to the —Ar substituent or adjacent to the point of attachment for the —Ar substituent;
Ar is aryl or heteroaryl;
R is —CH₃, —CH₂F, —CHF₂ or —CH═CH₂;
V is —H, —CH₃ or ═O;
W is —NO₂, —Cl, —Br, —CH₂OH, or —CN;
X is —Cl, —Br, —F, —CH₃, —OCH₃, or —CN;
Y is —CH or —N; and
Z is —CH or —N;
with the proviso that the compound of Formula I does not include the following compounds:

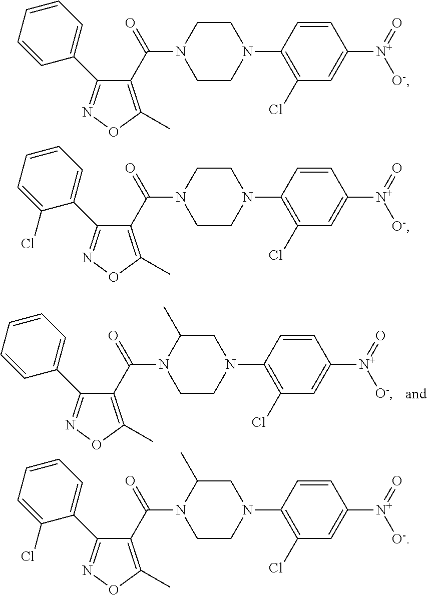

2. The compound of claim 1, wherein Het is selected from the group of:

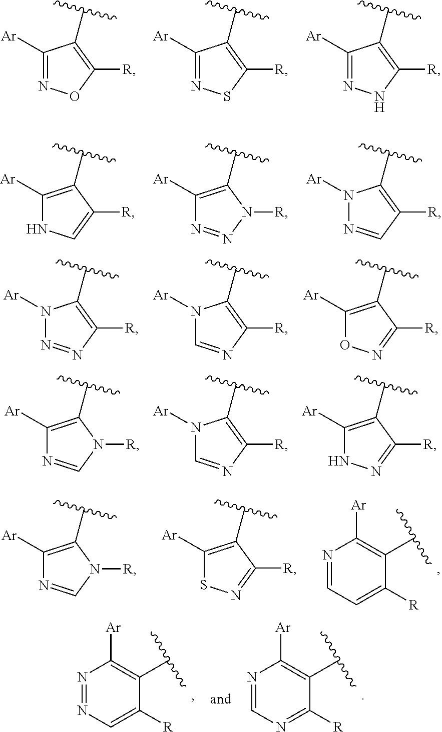

3. The compound of claim 2, wherein Het is a 5 or 6-membered heterocycle with —N adjacent to the point of attachment for the —Ar substituent.

4. The compound of claim 1, wherein Ar is selected from the group of: 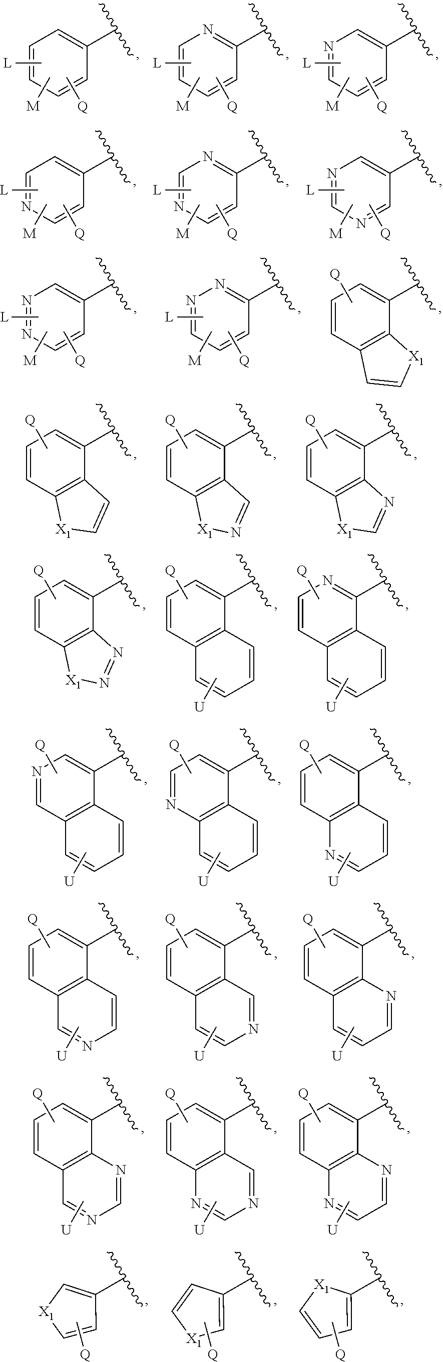 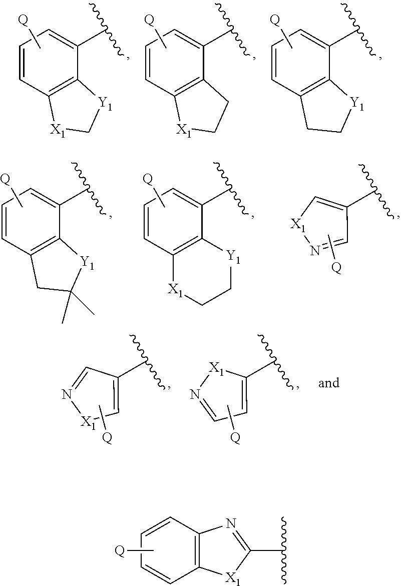
wherein
L is H, halogen, cyano, hydroxyl, amino, alkyl, alkoxy, alkylamino, or amido;
M is H, halogen, cyano, hydroxyl, amino, alkyl, alkoxy, alkylamino, or amido;
Q is H, halogen, cyano, hydroxyl, amino, alkyl, alkoxy, alkylamino, or amido;
U is H, halogen, cyano, hydroxyl, amino, alkyl, alkoxy, alkylamino, or amido;
X₁ is O, NH, N-alkyl, N-aryl, S or CH₂; and
Y₁ is O, NH, N-alkyl, N-aryl, S or CH₂.

5. The compound of claim 4, wherein Ar is phenyl.

6. The compound of claim 5, wherein Ar is phenyl substituted with methoxy or hydroxyl.

7. The compound of claim 1, wherein W is —NO₂, —Cl, —Br, or —CN.

8. The compound of claim 1, wherein X is —Cl or —CH₃.

9. The compound of claim 1, wherein Y is —CH or —N and Ar is phenyl substituted with methoxy or hydroxyl.

10. The compound of claim 1, wherein R is —CH₃ or —CH₂F.

11. The compound of claim 2, wherein Het is selected from the group of:

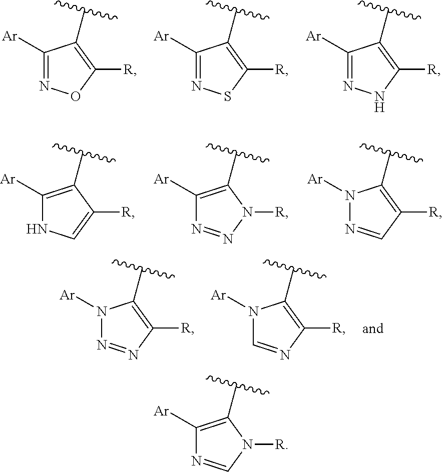

12. The compound of claim 4, wherein Ar is selected from the group of:

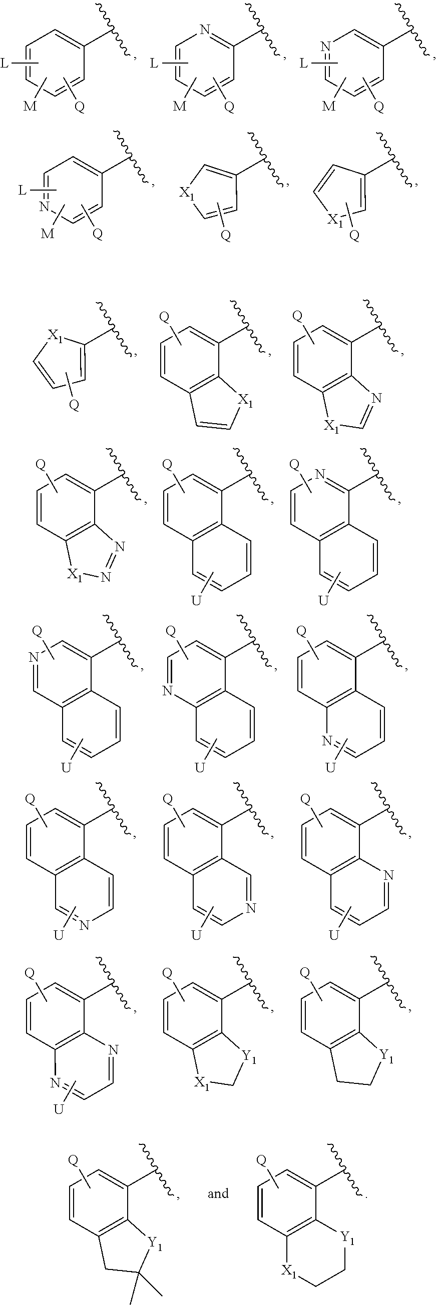

13. The compound of claim 7, wherein W is —NO₂, —Cl, or —Br.

14. The compound of claim 8, where X is —Cl.

15. The compound of claim 9, wherein Y is —CH or —N.

16. The compound of claim 10, wherein R is —CH₃.

17. The compound of claim 11, wherein Het is selected from the group of:

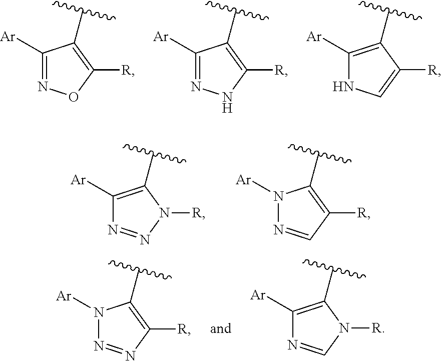

18. The compound of claim 12, wherein Ar is selected from the group of:

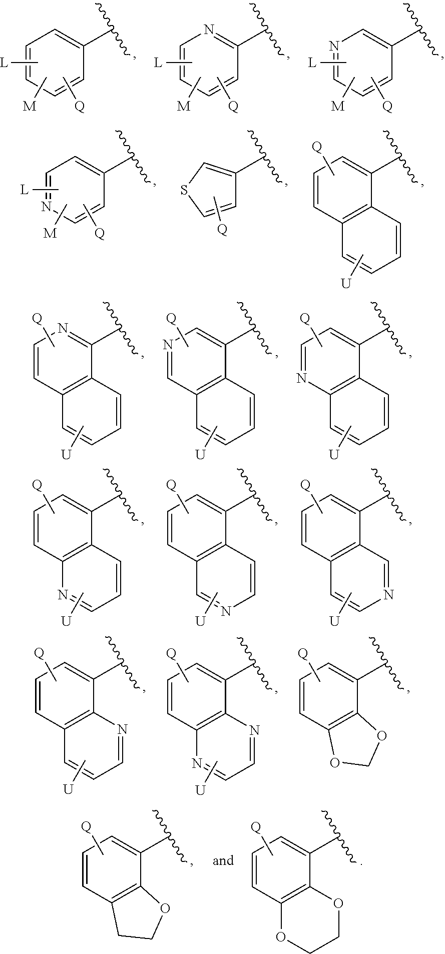

19. The compound of claim 18, wherein Ar is phenyl.

20. The compound of claim 19, wherein Ar is phenyl substituted with methoxy or hydroxyl.

21. The compound of claim 13, wherein W is —NO₂ or —Br.

22. The compound of claim 15, wherein Y is —CH.

23. A compound which is selected from the group consisting of:

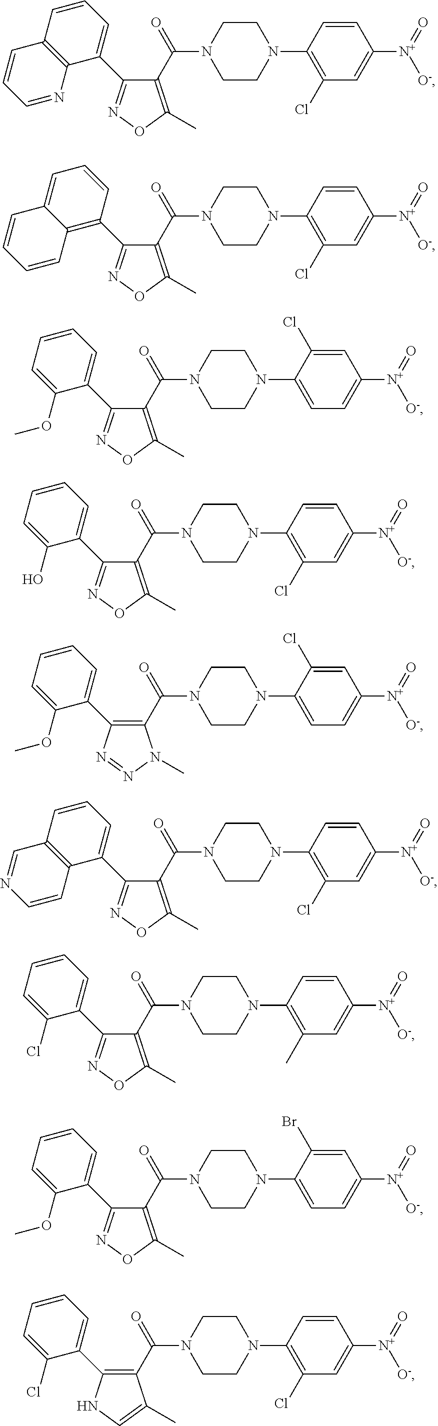

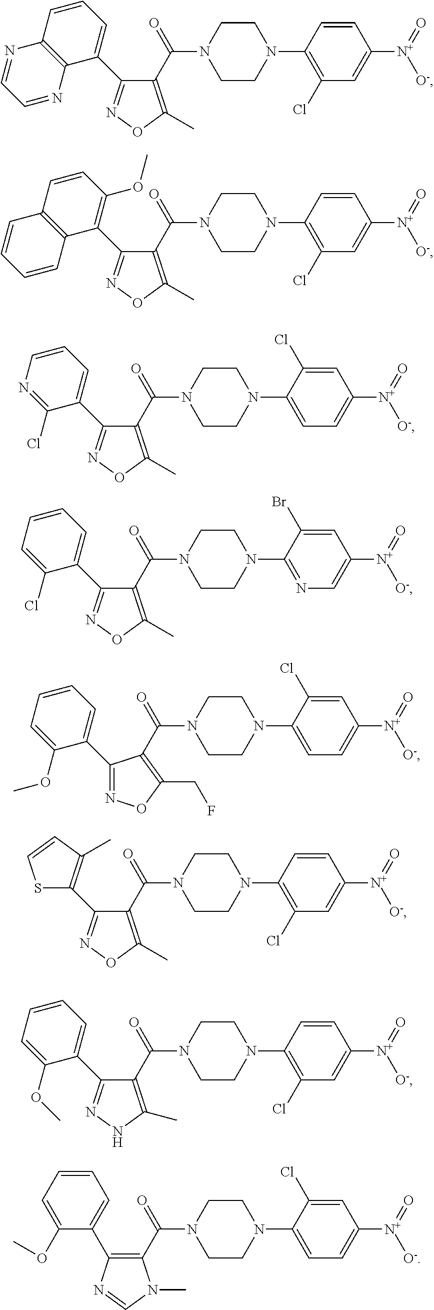

24. A pharmaceutical composition which comprises an antiviral effective amount of one or more of the compounds of Formula I as claimed in claim 1, together with one or more pharmaceutically acceptable carriers, excipients or diluents.

25. A method for treating a mammal infected with influenza virus comprising administering to said mammal an antiviral effective amount of a compound of Formula I as claimed in claim 1, and one or more pharmaceutically acceptable carriers, excipients or diluents.
